(19) United States
(12) Patent Application Publication
(10) Pub. No.: US 2025/0257077 A1
(43) Pub. Date: Aug. 14, 2025

(54) BRIDGED BICYCLIC HETEROCYCLOALKYL PYRIDO-[3,4-d]PYRIDAZINE AMINE DERIVATIVES USEFUL AS NLRP3 INHIBITORS

(71) Applicant: Ventus Therapeutics U.S., Inc., Waltham, MA (US)

(72) Inventors: Stéphane DORICH, Pointe-Claire (CA); Stéphane CIBLAT, Montreal (CA)

(21) Appl. No.: 19/186,473

(22) Filed: Apr. 22, 2025

Related U.S. Application Data
(63) Continuation of application No. PCT/US2023/078143, filed on Oct. 30, 2023.
(60) Provisional application No. 63/526,757, filed on Jul. 14, 2023.
(60) Provisional application No. 63/420,939, filed on Oct. 31, 2022.

(51) Int. Cl. C07D 519/00; A61K 31/5025; C07D 471/04

(52) U.S. Cl. CPC C07D 519/00; A61K 31/5025; C07D 471/04

(57) ABSTRACT

The present disclosure relates to compounds of Formula (I):

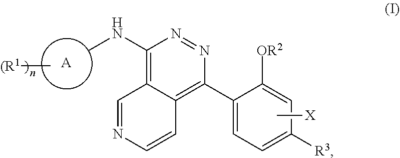

or pharmaceutically acceptable salts or isotopically labeled derivatives thereof, wherein A is a 6- to 10-membered bridged bicyclic heterocycloalkyl comprising at least one oxygen (O) ring atom, and R¹, R², R³, X and n as defined herein, useful in the treatment of diseases and disorders inhibited by said protein.

### CROSS-REFERENCE TO RELATED APPLICATIONS

This application is a continuation of International Application No. PCT/US2023/078143, filed Oct. 30, 2023, which claims the benefit of and priority to U.S. Provisional Patent Application Nos. 63/420,939, filed Oct. 31, 2022, and 63/526,757, filed Jul. 14, 2023, the entire contents of each of which are incorporated herein by reference.

### BACKGROUND

Innate immune responses are mediated by different types of receptors termed pattern-recognition receptors (PRRs). PRRs recognize the presence of pathogen-associated molecular patterns (PAMPs) and damage-associated molecular patterns (DAMPs) Once engaged these receptors trigger the activation of downstream inflammatory pathways that will help resolve injury. However, in many instances this activation can be uncontrolled and leads to disease.

The inflammasomes represent a class of PRRs that are crucial components of the innate immune response. Activation of the inflammasomes trigger a cascade of events that releases IL-1B. IL-18, and promotes an inflammatory form of cell death called pyroptosis induced by the activation of Gasdermin. Pyroptosis is a unique form of inflammatory cell death that leads to the release of not only cytokines but also other intracellular components that promote a broader immune response both of the innate and acquired immune system. Thus, inflammasome activation is a major regulatory of the inflammatory cascade.

NLRP3 is the most characterized inflammasome and has been shown to be critical in innate immunity and inflammatory responses. While several other NLR complexes, such as NLRC4, are activated under very specific circumstances. NLRP3 can be activated by numerous stimuli and should be seen as a sensor of intracellular homeostatic imbalance. Therefore, its precise functioning is essential. In addition to playing a role in host immune defense, dysregulation of NLRP3 has been linked to the pathogenesis of many inflammatory disorders. These include genetic diseases such as cryopyrin-associated periodic syndromes (CAPS) which is caused by gain-of-function mutations in the NLRP3 gene, as well as many prevalent neurologic and systemic diseases. Importantly, NLRP3 hyperactivation has been demonstrated pre-clinically to play a critical role in a plethora of inflammatory and degenerative diseases including. NASH, atherosclerosis and other cardiovascular diseases, Alzheimer's disease, Parkinson's disease, diabetes, gout, and numerous other autoinflammatory diseases. Thus, there is an unmet need in the field to develop small molecules for modulating NLRP3 activity to treat various diseases and disorders.

### SUMMARY

In one aspect, the present disclosure provides, inter alia, a compound of Formula (I):

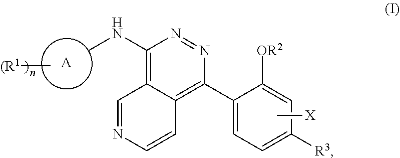

or a pharmaceutically acceptable salt or isotopically labeled derivative thereof, wherein:

A is a 6- to 10-membered bridged bicyclic heterocycloalkyl, wherein the heterocycloalkyl comprises at least one O ring atom;
each R¹ independently is halogen, C₁-C₆ alkyl, or C₁-C₆ alkoxy;
R² is H, C₁-C₆ alkyl, or —C(O)(C₁-C₆ alkyl);
R³ is —OH, halogen, —CN, C₁-C₆ alkyl, or C₁-C₆ alkoxy;
X is H, —OH, halogen, —NH₂, —NH(C₁-C₆ alkyl), —N(C₁-C₆ alkyl)₂, or C₁-C₆ alkyl; and
n is 0, 1, 2, 3, or 4,
   wherein each instance of alkyl or alk—is independently and optionally substituted with one or more halogen atoms.

In some aspects, the present disclosure provides pharmaceutical compositions comprising a compound of the present disclosure, or a pharmaceutically acceptable salt thereof, and one or more pharmaceutically acceptable excipients.

In some aspects, the present disclosure provides a method of treating a disease or disorder disclosed herein in a subject in need thereof, comprising administering to the subject a therapeutically effective amount of a compound of the present disclosure or a pharmaceutically acceptable salt or isotopically labeled derivative thereof, or a pharmaceutical composition of the present disclosure. In some embodiments, the subject is a human.

In other aspects, provided is a compound of the present disclosure or a pharmaceutically acceptable salt or isotopically labeled derivative thereof, or a pharmaceutical composition of the present disclosure for use in treating a disease or disorder.

In other aspects, provided is use of a compound of the present disclosure or a pharmaceutically acceptable salt or isotopically labeled derivative thereof, or a pharmaceutical composition of the present disclosure for treating a disease or disorder.

In other aspects, provided is use of a compound of the present disclosure or a pharmaceutically acceptable salt or isotopically labeled derivative thereof, or a pharmaceutical composition of the present disclosure in the manufacture of a medicament for treating a disease or disorder.

In some embodiments, the disease or disorder is an NLRP3-related disease or disorder. In some embodiments, the disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an NLRP3-related disease. In some embodiments, the disease or disorder of the central nervous system is Parkinson's disease. Alzheimer's disease, traumatic brain injury, spinal cord injury, amyotrophic lateral sclerosis, or multiple sclerosis. In some embodiments, the kidney disease is an acute kidney disease, a chronic kidney disease, or a rare kidney disease. In some embodiments, the skin disease is psoriasis, hidradenitis suppurativa (HS), or atopic dermatitis. In some embodiments, the rheumatic disease is dermatomyositis, Still's disease, or juvenile idiopathic arthritis. In some embodiments, the NLRP3-related disease is in a subject that has been determined to carry a germline or somatic non-silent mutation in NLRP3. In some embodiments, the NLRP3-related disease is in a subject that has been determined to carry a germline or somatic non-silent mutation in NLRP3 is cryopyrin-associated autoinflammatory syndrome. In some embodiments, the cryopyrin-associated autoinflammatory syndrome is familial cold autoinflammatory syndrome. Muckle-Wells syndrome, or neonatal onset multisystem inflammatory disease.

In some aspects, the present disclosure provides an intermediate as described herein, being suitable for use in a method for preparing a compound as described herein (e.g., the intermediate is selected from the intermediates described in Examples 1-12).

In some aspects, the present disclosure provides compounds obtainable by, or obtained by, a method for preparing a compound as described herein (e.g., a method comprising one or more steps described in General Synthetic Protocols A and B).

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

### (i) Definitions

Unless otherwise stated, the following terms used in the specification and claims have the following meanings set out below.

As used herein, “alkyl.” “C₁, C₂, C₃, C₄, C₅ or C₆ alkyl.” “C₁₋₆ alkyl,” or “C₁-C₆ alkyl” is intended to include C₁, C₂, C₃, C₄, C₅ or C₆ straight chain (linear) saturated aliphatic hydrocarbon groups and C₃. C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, I-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “6- to 10-membered bridged bicyclic heterocycloalkyl, wherein the heterocycloalkyl comprises at least one O ring atom,” refers to a saturated or partially unsaturated bridged bicyclic ring system having 1 or 2 oxygen (O) ring heteroatoms with the remaining ring atoms comprising carbon ring atoms, wherein the total number of ring atoms in the bridged bicyclic ring system comprising 6, 7, 8, 9, or 10 ring atom members. The bridged bicyclic ring system thus contemplated herein specifically excludes ring systems which are fused or spiro-fused, i.e., by requiring the two rings of the bicyclic heterocycloalkyl ring system (“bridged rings”) to share at least 3 or more ring atom members, and the two bridgehead ring atom members to be connected via a bridge containing at least one ring atom member. In some embodiments, the bridged bicyclic heterocycloalkyl is a fully saturated ring system. Exemplary saturated bridged bicyclic heterocycloalkyl groups comprising at least one ring O atom include, but are not limited to, 8-oxabicyclo[3.2.1]octanyl, 3-oxabicyclo[3.2.1]octanyl, 7-oxabicyclo[2.2.1]heptanyl, and 2-oxabicyclo[2.2.1]heptanyl. In some embodiments, the bridged bicyclic heterocycloalkyl ring system consists of two bridged rings which are 5-membered and 6-membered, wherein the point of attachment to Formula (I) is at any ring carbon atom. In some embodiments, the bridged bicyclic heterocycloalkyl ring system consists of two bridged rings which are 5-membered and 7-membered, wherein the point of attachment to Formula (I) is at any ring carbon atom. In some embodiments, the bridged bicyclic heterocycloalkyl consists of two bridged rings which are both 5-membered, wherein the point of attachment to Formula (I) is at any ring carbon atom. By way of example, an exemplary 3-oxabicyclo[3.2.1]octanyl radical

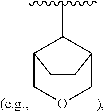

is a 6- to 10-membered bridged bicyclic heterocycloalkyl ring system consisting of two rings, with one ring comprising one oxygen ring heteroatom and with the remaining 7 ring atoms being carbon atoms, where the total number of ring atoms is 8. Such a ring system is not fused or spiro-fused.

As used herein, the term “halo” or “halogen” refers to fluoro, chloro, bromo and iodo.

The term “haloalkyl” refers to an alkyl as defined herein substituted with one or more halogen atoms. In some embodiments, all of the hydrogens of the alkyl group have been replaced with halogen atoms.

The term “alkoxy” or “alkoxyl” includes an alkyl group covalently linked by a direct bond to an oxygen atom, wherein the radical (point of attachment) is on the oxygen atom. Examples of alkoxy groups include, but are not limited to, methoxy, ethoxy, isopropyloxy, propoxy, butoxy and pentoxy groups. Examples of substituted alkoxy groups include halogenated alkoxy groups. Examples of halogen substituted alkoxy groups include, but are not limited to, fluoromethoxy, difluoromethoxy, trifluoromethoxy, chloromethoxy, dichloromethoxy and trichloromethoxy.

The term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” refers to a compound of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., gly cine, alanine, phenylalanine, arginine, etc.

Reference to a “salt” comprises any and all salts.

The term “isotopically labeled derivative,” as used herein, refers to a compound in which one or more atoms of the compound are provided as isotopically enriched or labeled atoms. In some embodiments, the isotopically labeled derivative is enriched with regard to, or labeled with, one or more atoms selected from ²H, ³H, ¹³C, ¹⁴C, ¹⁵N, ¹⁸O, or ¹⁸F. In some embodiments, the isotopically labeled derivative is a deuterium labeled compound (i.e., being enriched with ²H with regard to one or more hydrogen atoms thereof). In some embodiments, the isotopically labeled derivative is an ¹⁸F labeled compound (i.e., being enriched with ¹⁸F with regard to one or more fluorine atoms thereof). It is understood that the isotopically labeled derivative can be prepared using any of a variety of art-recognized techniques. For example, the isotopically labeled derivative can generally be prepared by carrying out the procedures disclosed in the Schemes and/or in the Examples described herein, by substituting an isotopically labeled reagent for a non-isotopically labeled reagent.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in some instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules.

As used herein, the term “subject” is interchangeable with the term “subject in need thereof,” both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. In one embodiment, the mammal is a human. A subject in need thereof can be one who has been previously diagnosed or identified as having a disease or disorder disclosed herein. A subject in need thereof can also be one who is suffering from a disease or disorder disclosed herein. A subject in need thereof can have a refractory or resistant disease or disorder disclosed herein (i.e., a disease or disorder disclosed herein that does not respond or has not yet responded to treatment). The subject may be resistant at the start of treatment or may become resistant during treatment. In some embodiments, the subject in need thereof received and failed all known effective therapies for a disease or disorder disclosed herein. In some embodiments, the subject in need thereof received at least one prior therapy. “Subject” and “patient” are used interchangeably herein.

As used herein, the term “treating” or “treat” describes the management and care of a patient for the purpose of combating a disease, condition, or disorder from which the patient is suffering from, and includes the administration of a compound of the present disclosure to alleviate one or more symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of one or more established symptoms of a condition. “Treating” or “treatment” of a state, disorder or condition therefore includes: inhibiting the state, disorder or condition, i.e., arresting, reducing or delaying the development of the disease or a relapse thereof (in case of maintenance treatment) or at least one clinical or subclinical symptom thereof, or relieving or attenuating the disease, i.e., causing regression of the state, disorder or condition or at least one of its clinical or subclinical symptoms.

As used herein, the term “therapeutically effective amount” or “effective amount,” which are used interchangeably herein, refers to an amount of a therapeutic agent, such as a compound of the present disclosure, to treat an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

### (ii) Compounds of the Present Disclosure

In one aspect, the present disclosure provides, inter alia, a compound of Formula (I):

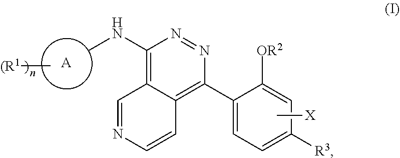

or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen, C₁-C₆ alkyl, or C₁-C₆ alkoxy;
R² is H, C₁-C₆ alkyl, or —C(O)(C₁-C₆ alkyl);
R³ is —OH, halogen, —CN, C₁-C₆ alkyl, or C₁-C₆ alkoxy;
X is H, —OH, halogen, —NH₂, —NH(C₁-C₆ alkyl), —N(C₁-C₆ alkyl) 2, or C₁-C₆ alkyl; and
n is 0, 1, 2, 3, or 4,
   wherein each instance of alkyl or alk—is independently and optionally substituted with one or more halogen atoms.

In some embodiments, the compound is of Formula (I), or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen. C₁-C₆ alkyl, or C₁-C₆ alkoxy;
R² is H, C₁-C₆ alkyl, or —C(O)(C₁-C₆ alkyl);
R³ is —OH, halogen, C₁-C₆ alkyl, or C₁-C₆ alkoxy;
X is H, —OH, halogen, —NH₂, —NH(C₁-C₆ alkyl), —N(C₁-C₆ alkyl) 2, or C₁-C₆ alkyl; and
n is 0, 1, 2, 3, or 4,
   wherein each instance of alkyl or alk—is independently substituted with 0, 1, 2, or 3 halogen atoms.

In some embodiments, the compound is of Formula (I), or a pharmaceutically acceptable salt or isotopically labeled derivative thereof, wherein:
A is a 6- to 8-membered bridged bicyclic heterocycloalkyl, wherein the heterocycloalkyl comprises at least one O ring atom;
R² is H;
R³ is halogen, C₁-C₆ haloalkyl, or C₁-C₆ alkyl;
X is H or halogen; and
n is 0.

In some embodiments, the compound is of Formula (I), or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen. C₁-C₆ alkyl, or C₁-C₆ alkoxy;
R² is H, C₁-C₆ alkyl, or —C(O)(C₁-C₆ alkyl);
R³ is —CN;
X is H, —OH, halogen, —NH₂, —NH(C₁-C₆ alkyl), —N(C₁-C₆ alkyl)₂, or C₁-C₆ alkyl; and
n is 0, 1, 2, 3, or 4,
   wherein each instance of alkyl or alk—is independently substituted with 0, 1, 2, or 3 halogen atoms.

In some embodiments, the compound is of Formula (I), or a pharmaceutically acceptable salt or isotopically labeled derivative thereof, wherein:
A is a 6- to 8-membered bridged bicyclic heterocycloalkyl, wherein the heterocycloalkyl comprises at least one O ring atom;
R² is H;

R³ is —CN; X is H or halogen; and n is 0.

In some embodiments, a compound of Formula (I), wherein R³ is an C₁-C₆ alkyl or C₁-C₆ alkoxy group, wherein each instance of alkyl or alk—is independently and optionally substituted with one or more halogen atom, may exhibit one or more desirable properties (e.g., solubility, NLRP3 potency, and/or stability) when compared to a compound of Formula (I), wherein R³ is a halogen group.

In some embodiments, such as development of a systemic (non-brain penetrant) inhibitor, a compound of Formula (I) wherein R³ is halogen, such as R³ is chloro, may be preferred.

In some embodiments, incorporation of a non-hydrogen X group ortho or meta to the —OR² moiety may result in an increase in metabolic stability. In some embodiments, incorporation of a fluoro X group ortho, meta, or para to the —OR² moiety may result in a compound that may be utilized for diagnostic purposes (e.g., as a positron emission tomography (PET) tracer wherein the fluoro group is ¹⁸F enriched). In some embodiments, incorporation of a fluoro group to a compound of Formula (I) may result in a compound that may be utilized for diagnostic purposes (e.g., as a positron emission tomography (PET) tracer when fluoro is ¹⁸F enriched).

### (i) Ring A, R¹, and n Embodiments

In some embodiments, A is a 6- to 10-membered bridged bicyclic heterocycloalkyl, wherein the heterocycloalkyl comprises at least one O ring atom. In some embodiments. A is a 6- to 10-membered bridged bicyclic heterocycloalkyl, wherein the heterocycloalkyl comprises one O ring atom.

In some embodiments, A is a 6-membered bridged bicyclic heterocycloalkyl, wherein the heterocycloalkyl comprises at least one O ring atom. In some embodiments, A is a 6-membered bridged bicyclic heterocycloalkyl, wherein the heterocycloalkyl comprises one O ring atom.

In some embodiments, A is a 7-membered bridged bicyclic heterocycloalkyl, wherein the heterocycloalkyl comprises at least one O ring atom. In some embodiments, A is a 7-membered bridged bicyclic heterocycloalkyl, wherein the heterocycloalkyl comprises one O ring atom.

In some embodiments, A is a 8-membered bridged bicyclic heterocycloalkyl, wherein the heterocycloalkyl comprises at least one O ring atom. In some embodiments. A is a 8-membered bridged bicyclic heterocycloalkyl, wherein the heterocycloalkyl comprises one O ring atom.

In some embodiments, A is a 9-membered bridged bicyclic heterocycloalkyl, wherein the heterocycloalkyl comprises at least one O ring atom. In some embodiments, A is a 9-membered bridged bicyclic heterocycloalkyl, wherein the heterocycloalkyl comprises one O ring atom.

In some embodiments, A is a 10-membered bridged bicyclic heterocycloalkyl, wherein the heterocycloalkyl comprises at least one O ring atom. In some embodiments, A is a 10-membered bridged bicyclic heterocycloalkyl, wherein the heterocycloalkyl comprises one O ring atom.

In some embodiments, A is a bridged bicyclic heterocycloalkyl ring system comprising two bridged rings which are 5-membered and 6-membered, wherein the point of attachment is at any ring carbon atom. In some embodiments, A is a bridged bicyclic heterocycloalkyl ring system comprising two bridged rings which are 5-membered and 7-membered, wherein the point of attachment is at any ring carbon atom. In some embodiments. A is a bridged bicyclic heterocycloalkyl comprising two bridged rings which are both 5-membered, wherein the point of attachment is at any ring carbon atom. In some embodiments, Ring A is a fully saturated bridged bicyclic heterocycloalkyl ring system.

In some embodiments, A is a fully saturated bridged bicyclic ring system of formula

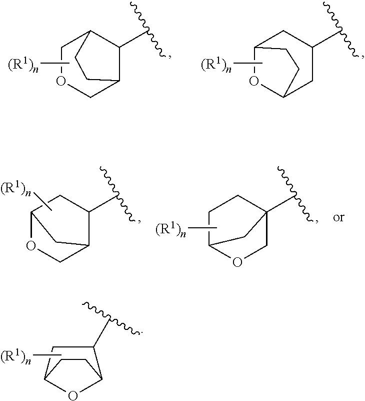

It is understood that R¹ may be substituted at any carbon atom on the bicyclic ring.

In some embodiments, A is

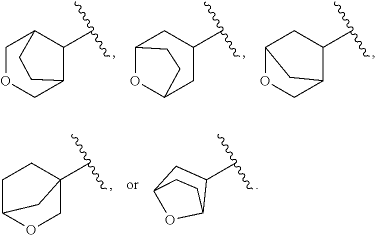

In some embodiments, A is

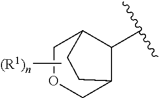

In some embodiments, A is

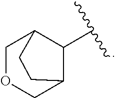 [SMILES: *C1C2CCC1COC2]

In some embodiments, A is

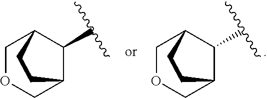

In some embodiments, A is

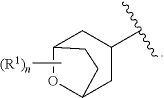

In some embodiments, A is

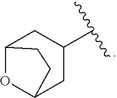 [SMILES: *C1CC2CCC(C1)O2]

In some embodiments, A is

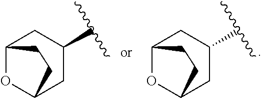 [SMILES: *[C@H]1C[C@H]2CC[C@@H](C1)O2 ; *[C@@H]1C[C@H]2CC[C@@H](C1)O2]

In some embodiments, A is

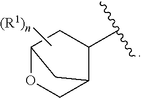

In some embodiments, A is

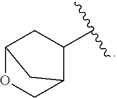 [SMILES: *C1CC2CC1CO2]

In some embodiments, A is

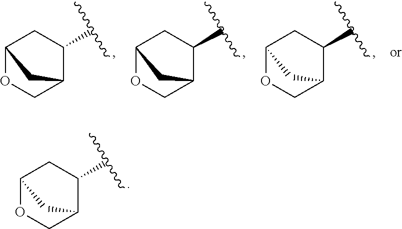

In some embodiments, A is

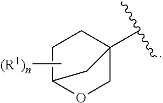

In some embodiments, A is

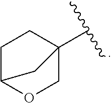 [SMILES: *C12CCC(C1)OC2]

In some embodiments, A is

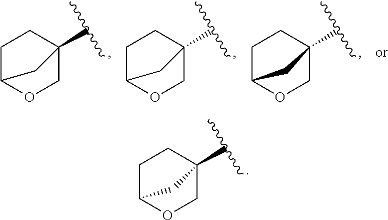

However, in some embodiments, A is not

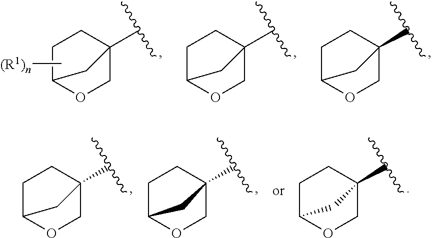

In some embodiments, A is

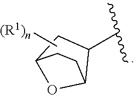

In some embodiments, A is

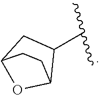 [SMILES: *C1CC2CCC1O2]

In some embodiments, A is

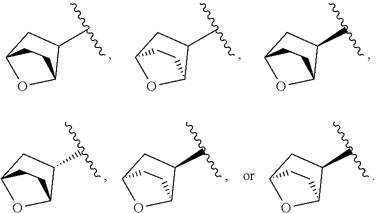 [SMILES: *C1C[C@@H]2CC[C@H]1O2 ; *C1C[C@H]2CC[C@@H]1O2 ; *[C@@H]1C[C@@H]2CC[C@H]1O2 ; *[C@H]1C[C@@H]2CC[C@H]1O2 ; *[C@@H]1C[C@H]2CC[C@@H]1O2 ; *[C@@H]1C[C@H]2CC[C@@H]1O2]

In some embodiments, n is 0, 1, 2, 3, or 4.

In some embodiments, n is 0. In some embodiments, n is 1. In some embodiments, n is 2. In some embodiments, n is 3. In some embodiments, n is 4.

In some embodiments, each R¹ independently is halogen, C₁-C₆ alkyl, or C₁-C₆ alkoxy, wherein the alkyl or alkoxy is optionally and independently substituted with one or more halogen atoms. In some embodiments, each hydrogen of the alkyl or alkoxy group is replaced with a halogen atom. In some embodiments, at least one hydrogen of the alkyl or alkoxy group is replaced with a halogen atom. In some embodiments, the alkyl or alkoxy is substituted with 0, 1, 2, or 3 halogen atoms.

In some embodiments, each R¹ independently is halogen. C₁-C₆ alkyl, or C₁-C₆ alkoxy, wherein the alkyl or alkoxy is substituted with 1, 2, or 3 halogen atoms.

In some embodiments, each R¹ independently is halogen. C₁-C₆ alkyl, or C₁-C₆ alkoxy.

In some embodiments, at least one R¹ is halogen. In some embodiments, each R¹ independently is halogen.

In some embodiments, at least one R¹ is F, Cl, Br, or I. In some embodiments, each R¹ independently is F, Cl, Br, or I.

In some embodiments, each R¹ independently is F. In some embodiments, each R¹ independently is C₁. In some embodiments, each R¹ independently is Br. In some embodiments, each R¹ independently is 1.

In some embodiments, at least one R¹ is C₁-C₆ alkyl optionally substituted with one or more halogen. In some embodiments, each R¹ independently is C₁-C₈ alkyl substituted with 0, 1, 2, or 3 halogen.

In some embodiments, at least one R¹ is C₁-C₆ alkyl substituted with one or more halogen. In some embodiments, each R¹ independently is C₁-C₆ alkyl substituted with 1, 2, or 3 halogen.

In some embodiments, at least one R¹ is C₁-C₆ alkyl. In some embodiments, each R¹ independently is C₁-C₆ alkyl.

In some embodiments, at least one R¹ is methyl optionally substituted with one or more halogen. In some embodiments, at least one R¹ is ethyl optionally substituted with one or more halogen. In some embodiments, at least one R¹ is propyl optionally substituted with one or more halogen. In some embodiments, at least one R¹ is butyl optionally substituted with one or more halogen. In some embodiments, at least one R¹ is pentyl optionally substituted with one or more halogen. In some embodiments, at least one R¹ is hexyl optionally substituted with one or more halogen. In some embodiments, at least one R¹ is isopropyl optionally substituted with one or more halogen. In some embodiments, at least one R¹ is isobutyl optionally substituted with one or more halogen. In some embodiments, at least one R¹ is isopentyl optionally substituted with one or more halogen. In some embodiments, at least one R¹ is isohexyl optionally substituted with one or more halogen. In some embodiments, at least one R¹ is secbutyl optionally substituted with one or more halogen. In some embodiments, at least one R¹ is secpentyl optionally substituted with one or more halogen. In some embodiments, at least one R¹ is sechexyl optionally substituted with one or more halogen. In some embodiments, at least one R¹ is tertbutyl optionally substituted with one or more halogen.

In some embodiments, each R¹ independently is methyl substituted with 0, 1, 2, or 3 halogen. In some embodiments, each R¹ independently is ethyl substituted with 0, 1, 2, or 3 halogen. In some embodiments, each R¹ independently is propyl substituted with 0, 1, 2, or 3 halogen. In some embodiments, each R¹ independently is butyl substituted with 0, 1, 2, or 3 halogen. In some embodiments, each R¹ independently is pentyl substituted with 0, 1, 2, or 3 halogen. In some embodiments, each R¹ independently is hexyl substituted with 0, 1, 2, or 3 halogen. In some embodiments, each R¹ independently is isopropyl substituted with 0, 1, 2, or 3 halogen. In some embodiments, each R¹ independently is isobutyl substituted with 0, 1, 2, or 3 halogen. In some embodiments, each R¹ independently is isopentyl substituted with 0, 1, 2, or 3 halogen. In some embodiments, each R¹ independently is isohexyl substituted with 0, 1, 2, or 3 halogen. In some embodiments, each R¹ independently is secbutyl substituted with 0, 1, 2, or 3 halogen. In some embodiments, each R independently is secpentyl substituted with 0, 1, 2, or 3 halogen. In some embodiments, each R¹ independently is sechexyl substituted with 0, 1, 2, or 3 halogen. In some embodiments, each R¹ independently is tertbutyl substituted with 0, 1, 2, or 3 halogen.

In some embodiments, each R¹ independently is methyl substituted with 1, 2, or 3 halogen. In some embodiments, each R¹ independently is ethyl substituted with 1, 2, or 3 halogen. In some embodiments, each R¹ independently is propyl substituted with 1, 2, or 3 halogen. In some embodiments, each R¹ independently is butyl substituted with 1, 2, or 3 halogen. In some embodiments, each R¹ independently is pentyl substituted with 1, 2, or 3 halogen. In some embodiments, each R¹ independently is hexyl substituted with 1, 2, or 3 halogen. In some embodiments, each R¹ independently is isopropyl substituted with 1, 2, or 3 halogen. In some embodiments, each R¹ independently is isobutyl substituted with 1, 2, or 3 halogen. In some embodiments, each R¹ independently is isopentyl substituted with 1, 2, or 3 halogen. In some embodiments, each R¹ independently is isohexyl substituted with 1, 2, or 3 halogen. In some embodiments, each R¹ independently is secbutyl substituted with 1, 2, or 3 halogen. In some embodiments, each R¹ independently is secpentyl substituted with 1, 2, or 3 halogen. In some embodiments, each R¹ independently is sechexyl substituted with 1, 2, or 3 halogen. In some embodiments, each R¹ independently is tertbutyl substituted with 1, 2, or 3 halogen.

In some embodiments, each R¹ independently is methyl. In some embodiments, each R¹ independently is ethyl. In some embodiments, each R¹ independently is propyl. In some embodiments, each R¹ independently is butyl. In some embodiments, each R¹ independently is pentyl. In some embodiments, each R¹ independently is hexyl. In some embodiments, each R¹ independently is isopropyl. In some embodiments, each R¹ independently is isobutyl. In some embodiments, each R¹ independently is isopentyl. In some embodiments, each R¹ independently is isohexyl. In some embodiments, each R¹ independently is secbutyl. In some embodiments, each R¹ independently is secpentyl. In some embodiments, each R¹ independently is sechexyl. In some embodiments, each R¹ independently is tertbutyl.

In some embodiments, at least one R¹ is C₁-C₆ alkoxy optionally substituted with one or more halogen. In some embodiments, each R¹ independently is C₁-C₆ alkoxy substituted with 0, 1, 2, or 3 halogen.

In some embodiments, at least one R¹ is C₁-C₆ alkoxy substituted with one or more halogen. In some embodiments, each R¹ independently is C₁-C₆ alkoxy substituted with 1, 2, or 3 halogen. In some embodiments, each R¹ independently is C₁-C₆ alkoxy.

In some embodiments, at least one R¹ is C₁ alkoxy optionally substituted with one or more halogen. In some embodiments, at least one R¹ is C₂ alkoxy optionally substituted with one or more halogen. In some embodiments, at least one R¹ is C₃ alkoxy optionally substituted with one or more halogen. In some embodiments, at least one R¹ is C₄ alkoxy optionally substituted with one or more halogen. In some embodiments, at least one R¹ is C₅ optionally substituted with one or more halogen. In some embodiments, at least one R¹ is C₆ alkoxy optionally substituted with one or more halogen.

In some embodiments, each R¹ independently is C₁ alkoxy substituted with 0, 1, 2, or 3 halogen. In some embodiments, each R¹ independently is C₂ alkoxy substituted with 0, 1, 2, or 3 halogen. In some embodiments, each R¹ independently is C₃ alkoxy substituted with 0, 1, 2, or 3 halogen. In some embodiments, each R¹ independently is C₄ alkoxy substituted with 0, 1, 2, or 3 halogen. In some embodiments, each R¹ independently is C₅ alkoxy substituted with 0, 1, 2, or 3 halogen. In some embodiments, each R¹ independently is C₆ alkoxy substituted with 0, 1, 2, or 3 halogen.

In some embodiments, each R¹ independently is C₁ alkoxy substituted with 1, 2, or 3 halogen. In some embodiments, each R¹ independently is C₂ alkoxy substituted with 1, 2, or 3 halogen. In some embodiments, each R¹ independently is C₃ alkoxy substituted with 1, 2, or 3 halogen. In some embodiments, each R¹ independently is C₄ alkoxy substituted with 1, 2, or 3 halogen. In some embodiments, each R¹ independently is C₅ alkoxy substituted with 1, 2, or 3 halogen. In some embodiments, each R¹ independently is C₆ alkoxy substituted with 1, 2, or 3 halogen.

In some embodiments, each R¹ independently is C₁ alkoxy. In some embodiments, each R¹ independently is C₂ alkoxy. In some embodiments, each R¹ independently is C₅ alkoxy. In some embodiments, each R¹ independently is C₄ alkoxy. In some embodiments, each R¹ independently is C₅ alkoxy. In some embodiments, each R¹ independently is Ce alkoxy.

### (ii) R², R³ and X Embodiments

In some embodiments, R² is H, C₁-C₆ alkyl, or —C(O)(C₁-C₆ alkyl), wherein alkyl is optionally substituted with one or more halogen atoms. In some embodiments, the alkyl group as recited above is substituted with 0, 1, 2, or 3 halogen atoms.

In some embodiments. R² is H.

In some embodiments. R² is C₁-C₆ alkyl optionally substituted with one or more halogen atoms. In some embodiments, R² is C₁—C& alkyl substituted with 0, 1, 2, or 3 halogen.

In some embodiments. R² is methyl. In some embodiments, R² is ethyl. In some embodiments. R² is propyl. In some embodiments, R² is butyl. In some embodiments, R² is pentyl. In some embodiments, R² is hexyl. In some embodiments, each R² is isopropyl. In some embodiments. R² is isobutyl. In some embodiments. R² is isopentyl. In some embodiments. R² is isohexyl. In some embodiments, R² is secbutyl. In some embodiments. R² is secpentyl. In some embodiments. R² is sechexyl. In some embodiments, R² is tertbutyl. In some embodiments, the alkyl group as recited above is optionally substituted with one or more halogen. In some embodiments, the alkyl group as recited above is substituted with one or more halogen. In some embodiments, the alkyl group as recited above is substituted with 0, 1, 2, or 3 halogen. In some embodiments, the alkyl group as recited above is substituted with 1, 2, or 3 halogen.

In some embodiments, R² is —C(O)(C₁-C₆ alkyl), wherein the alkyl is optionally substituted with one or more halogen atoms.

In some embodiments, R² is —C(O)(C₁-C₆ alkyl), wherein the alkyl is substituted with 0, 1, 2, or 3 halogen.

In some embodiments, R² is —C(O)(C₁ alkyl). In some embodiments, R² is —C(O)(C₂ alkyl). In some embodiments, R² is —C(O)(C₃ alkyl). In some embodiments. R² is —C(O)(C₄ alkyl). In some embodiments, R² is —C(O)(C₅ alkyl) In some embodiments, R² is —C(O)(C₆ alkyl).

In some embodiments. R³ is —OH, halogen, —CN, C₁-C₆ alkyl, or C₁-C₆ alkoxy, wherein the alkyl or alkoxy is optionally substituted with one or more halogen. In some embodiments, R³ is —OH, halogen, —CN, C₁-C₆ alkyl, or C₁-C₆ alkoxy, wherein the alkyl or alkoxy is substituted with 0, 1, 2, or 3 halogen.

In some embodiments, R³ is —OH, halogen, C₁-C₆ alkyl, or C₁-C₆ alkoxy, wherein the alkyl or alkoxy is substituted with one or more halogen. In some embodiments, R³ is —OH, halogen, C₁-C₆ alkyl, or C₁-C₆ alkoxy, wherein the alkyl or alkoxy is substituted with 1, 2, or 3 halogen.

In some embodiments. R³ is —OH, halogen, C₁-C₆ alkyl, or C₁-C₆ alkoxy.

In some embodiments. R³ is —OH.

In some embodiments, R³ is halogen.

In some embodiments, R³ is Br. In some embodiments, R³ is Cl. In some embodiments, R³ is F. In some embodiments. R³ is I.

In some embodiments, R³ is —CN.

In some embodiments, R³ is C₁-C₆ alkyl optionally substituted with one or more halogen. In some embodiments. R³ is C₁-C₆ alkyl substituted with 0, 1, 2, or 3 halogen.

In some embodiments, R³ is C₁-C₆ alkyl substituted with one or more halogen. In some embodiments, R³ is C₁-C₆ alkyl substituted with 1, 2, or 3 halogen. In such instances, R³ is interchangeably and collectively referred to as C₁-C₆ haloalkyl.

In some embodiments, R³ is C₁-C₆ alkyl.

In some embodiments, R³ is methyl optionally substituted with one or more halogen. In some embodiments. R³ is ethyl optionally substituted with one or more halogen. In some embodiments, R³ is propyl optionally substituted with one or more halogen. In some embodiments. R³ is butyl optionally substituted with one or more halogen. In some embodiments. R³ is pentyl optionally substituted with one or more halogen. In some embodiments, R³ is hexyl optionally substituted with one or more halogen. In some embodiments. R³ is isopropyl optionally substituted with one or more halogen. In some embodiments, R³ is isobutyl optionally substituted with one or more halogen. In some embodiments, R³ is isopentyl optionally substituted with one or more halogen. In some embodiments, R³ is isohexyl optionally substituted with one or more halogen. In some embodiments, R³ is secbutyl optionally substituted with one or more halogen. In some embodiments. R³ is secpentyl optionally substituted with one or more halogen. In some embodiments. R³ is sechexyl optionally substituted with one or more halogen. In some embodiments. R³ is tertbutyl optionally substituted with one or more halogen. In some embodiments. R³ is C₁-C₆ haloalkyl.

In some embodiments. R³ is C; haloalkyl. In some embodiments. R³ is C; haloalkyl. In some embodiments. R³ is C₃ haloalkyl. In some embodiments, R³ is C₄ haloalkyl. In some embodiments. R³ is C₅ haloalkyl. In some embodiments, R³ is C₆ haloalkyl.

In some embodiments. R³ is methyl substituted with 0, 1, 2, or 3 halogen. In some embodiments, R³ is ethyl substituted with 0, 1, 2, or 3 halogen. In some embodiments, R³ is propyl substituted with 0, 1, 2, or 3 halogen. In some embodiments. R³ is butyl substituted with 0, 1, 2, or 3 halogen. In some embodiments. R³ is pentyl substituted with 0, 1, 2, or 3 halogen. In some embodiments. R³ is hexyl substituted with 0, 1, 2, or 3 halogen. In some embodiments, R³ is isopropyl substituted with 0, 1, 2, or 3 halogen. In some embodiments. R³ is isobutyl substituted with 0, 1, 2, or 3 halogen. In some embodiments, R³ is isopentyl substituted with 0, 1, 2, or 3 halogen. In some embodiments, R³ is isohexyl substituted with 0, 1, 2, or 3 halogen. In some embodiments. R³ is secbutyl substituted with 0, 1, 2, or 3 halogen. In some embodiments, R³ is secpentyl substituted with 0, 1, 2, or 3 halogen. In some embodiments. R³ is sechexyl substituted with 0, 1, 2, or 3 halogen. In some embodiments, R³ is tertbutyl substituted with 0, 1, 2, or 3 halogen.

In some embodiments, R³ is methyl substituted with 1, 2, or 3 halogen. In some embodiments, R³ is ethyl substituted with 1, 2, or 3 halogen. In some embodiments. R³ is propyl substituted with 1, 2, or 3 halogen. In some embodiments. R³ is butyl substituted with 1, 2, or 3 halogen. In some embodiments. R³ is pentyl substituted with 1, 2, or 3 halogen. In some embodiments, R³ is hexyl substituted with 1, 2, or 3 halogen. In some embodiments, R³ is isopropyl substituted with 1, 2, or 3 halogen. In some embodiments, R³ is isobutyl substituted with 1, 2, or 3 halogen. In some embodiments. R³ is isopentyl substituted with 1, 2, or 3 halogen. In some embodiments. R³ is isohexyl substituted with 1, 2, or 3 halogen. In some embodiments. R³ is secbutyl substituted with 1, 2, or 3 halogen. In some embodiments, R³ is secpentyl substituted with 1, 2, or 3 halogen. In some embodiments. R³ is sechexyl substituted with 1, 2, or 3 halogen. In some embodiments. R³ is tertbutyl substituted with 1, 2, or 3 halogen.

In some embodiments, R³ is methyl. In some embodiments, R³ is ethyl. In some embodiments, R³ is propyl. In some embodiments. R³ is butyl. In some embodiments, R³ is pentyl. In some embodiments. R³ is hexyl. In some embodiments. R³ is isopropyl. In some embodiments, R³ is isobutyl. In some embodiments, R³ is isopentyl. In some embodiments, R³ is isohexyl. In some embodiments. R³ is secbutyl. In some embodiments, R³ is secpentyl. In some embodiments. R³ is sechexyl. In some embodiments. R³ is tertbutyl.

In some embodiments, R³ is C₁-C₆ alkoxy optionally substituted with one or more halogen. In some embodiments, R³ is C₁-C₆ alkoxy substituted with 0, 1, 2, or 3 halogen.

In some embodiments, R³ is C₁-C₆ alkoxy substituted with one or more halogen. In some embodiments, R³ is C₁-C₆ alkoxy substituted with 1, 2, or 3 halogen.

In some embodiments. R³ is C₁-C₆ alkoxy.

In some embodiments. R³ is methoxy optionally substituted with one or more halogen. In some embodiments, R³ is ethoxy optionally substituted with one or more halogen. In some embodiments, R³ is propoxy optionally substituted with one or more halogen. In some embodiments. R³ is butoxy optionally substituted with one or more halogen. In some embodiments. R³ is pentoxy optionally substituted with one or more halogen. In some embodiments. R³ is hexoxy optionally substituted with one or more halogen.

In some embodiments, R³ is methoxy substituted with 0, 1, 2, or 3 halogen. In some embodiments, R³ is ethoxy substituted with 0, 1, 2, or 3 halogen. In some embodiments. R³ is propoxy substituted with 0, 1, 2, or 3 halogen. In some embodiments, R³ is butoxy substituted with 0, 1, 2, or 3 halogen. In some embodiments, R³ is pentoxy substituted with 0, 1, 2, or 3 halogen. In some embodiments, R³ is hexoxy substituted with 0, 1, 2, or 3 halogen.

In some embodiments, R³ is methoxy substituted with 1, 2, or 3 halogen. In some embodiments, R³ is ethoxy substituted with 1, 2, or 3 halogen. In some embodiments, R³ is propoxy substituted with 1, 2, or 3 halogen. In some embodiments, R³ is butoxy substituted with 1, 2, or 3 halogen. In some embodiments, R³ is pentoxy substituted with 1, 2, or 3 halogen. In some embodiments. R³ is hexoxy substituted with 1, 2, or 3 halogen.

In some embodiments, R³ is methoxy. In some embodiments, R³ is ethoxy. In some embodiments, R³ is propoxy. In some embodiments. R³ is butoxy. In some embodiments, R³ is pentoxy. In some embodiments, R³ is hexoxy.

In some embodiments. R³ is —CF₃, —CHF₂, or —OCHF₂.

In some embodiments. R³ is —CF₃. In some embodiments, R³ is —CHF₂. In some embodiments, R³ is —OCHF₂.

In some embodiments, X is H, —OH, halogen, —NH₂, —NH(C₁-C₆ alkyl), —N(C₁-C₆ alkyl)₂, or C₁-C₆ alkyl, wherein alkyl is optionally and independently substituted with one or more halogen atoms. In some embodiments, the alkyl group as recited above is independently substituted with 0, 1, 2, or 3 halogen atoms.

In some embodiments, X is —OH, halogen, —NH₂·-NH(C₁-C₆ alkyl), —N(C₁-C₆ alkyl)₂, or C₁-C₆ alkyl, wherein alkyl is optionally and independently substituted with one or more halogen atoms. In some embodiments, the alkyl group as recited above is independently substituted with 0, 1, 2, or 3 halogen atoms. In some embodiments, X is a non-hydrogen group as listed above, and is located at the ortho position relative to the —OR² group, as depicted below. In some embodiments, X is a non-hydrogen group as listed above, and is located at the meta position relative to the —OR² group, as depicted below. In some embodiments, X is a non-hydrogen group as listed above, and is located at the para position relative to the —OR² group, as depicted below.

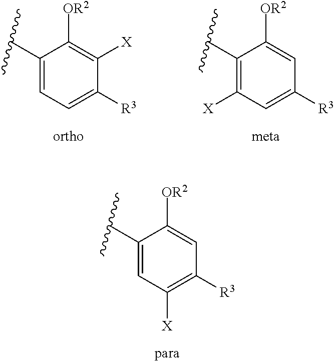

In some embodiments, X is H.

In some embodiments, X is halogen.

In some embodiments, X is Br, Cl, F, or I.

In some embodiments, X is Br. In some embodiments, X is Cl. In some embodiments, X is F. In some embodiments, X is 1.

In some embodiments, X is —OH.

In some embodiments, X is —NH₂. In some embodiments, X is —NH(C₁-C₆ alkyl), wherein alkyl is optionally and independently substituted with one or more halogen atoms. In some embodiments, the alkyl group as recited above is independently substituted with 0, 1, 2, or 3 halogen atoms.

In some embodiments, X is —NH(C; alkyl). In some embodiments, X is —NH(C₂ alkyl). In some embodiments, X is —NH(C; alkyl). In some embodiments, X is —NH(C₄ alkyl). In some embodiments, X is —NH(C₅ alkyl). In some embodiments, X is —NH(C₆ alkyl).

In some embodiments, X is —N(C₁-C₆ alkyl); wherein alkyl is optionally and independently substituted with one or more halogen atoms. In some embodiments, the alkyl group as recited above is optionally and independently substituted with 0, 1, 2, or 3 halogen atoms.

In some embodiments, X is —N(C₁ alkyl)₂. In some embodiments, X is —N(C₂ alkyl)₂. In some embodiments, X is —N(C₃ alkyl)₂. In some embodiments, X is —N(C₄ alkyl)₂. In some embodiments, X is —N(C₅ alkyl)₂. In some embodiments, X is —N(C₆ alkyl)₂.

In some embodiments, X is C₁-C₆ alkyl, wherein alkyl is optionally substituted with one or more halogen atoms. In some embodiments, the alkyl group as recited above is substituted with 0, 1, 2, or 3 halogen atoms.

In some embodiments, X is methyl optionally substituted with one or more halogen atoms. In some embodiments, X is ethyl optionally substituted with one or more halogen atoms. In some embodiments, X is propyl optionally substituted with one or more halogen atoms. In some embodiments, X is butyl optionally substituted with one or more halogen atoms. In some embodiments, X is pentyl optionally substituted with one or more halogen atoms. In some embodiments, X is hexyl optionally substituted with one or more halogen atoms. In some embodiments, each X is isopropyl optionally substituted with one or more halogen atoms. In some embodiments, X is isobutyl optionally substituted with one or more halogen atoms. In some embodiments, X is isopentyl optionally substituted with one or more halogen atoms. In some embodiments, X is isohexyl optionally substituted with one or more halogen atoms. In some embodiments, X is secbutyl optionally substituted with one or more halogen atoms. In some embodiments, X is secpentyl optionally substituted with one or more halogen atoms. In some embodiments, X is sechexyl optionally substituted with one or more halogen atoms. In some embodiments, X is tertbutyl optionally substituted with one or more halogen atoms.

In some embodiments, X is methyl substituted with 0, 1, 2, or 3 halogen atoms. In some embodiments, X is ethyl substituted with 0, 1, 2, or 3 halogen atoms. In some embodiments, X is propyl substituted with 0, 1, 2, or 3 halogen atoms. In some embodiments, X is butyl substituted with 0, 1, 2, or 3 halogen atoms. In some embodiments, X is pentyl substituted with 0, 1, 2, or 3 halogen atoms. In some embodiments, X is hexyl substituted with 0, 1, 2, or 3 halogen atoms. In some embodiments, each X is isopropyl substituted with 0, 1, 2, or 3 halogen atoms. In some embodiments, X is isobutyl substituted with 0, 1, 2, or 3 halogen atoms. In some embodiments, X is isopentyl substituted with 0, 1, 2, or 3 halogen atoms. In some embodiments, X is isohexyl substituted with 0, 1, 2, or 3 halogen atoms. In some embodiments, X is secbutyl substituted with 0, 1, 2, or 3 halogen atoms. In some embodiments, X is secpentyl substituted with 0, 1, 2, or 3 halogen atoms. In some embodiments, X is substituted with 0, 1, 2, or 3 sechexyl. In some embodiments, X is tertbutyl substituted with 0, 1, 2, or 3 halogen atoms.

In some embodiments, X is methyl substituted with 1, 2, or 3 halogen atoms. In some embodiments, X is ethyl substituted with 1, 2, or 3 halogen atoms. In some embodiments, X is propyl substituted with 1, 2, or 3 halogen atoms. In some embodiments, X is butyl substituted with 1, 2, or 3 halogen atoms. In some embodiments, X is pentyl substituted with 1, 2, or 3 halogen atoms. In some embodiments, X is hexyl substituted with 1, 2, or 3 halogen atoms. In some embodiments, each X is isopropyl substituted with 1, 2, or 3 halogen atoms. In some embodiments, X is isobutyl substituted with 1, 2, or 3 halogen atoms. In some embodiments, X is isopentyl substituted with 1, 2, or 3 halogen atoms. In some embodiments, X is isohexyl substituted with 1, 2, or 3 halogen atoms. In some embodiments, X is secbutyl substituted with 1, 2, or 3 halogen atoms. In some embodiments, X is secpentyl substituted with 1, 2, or 3 halogen atoms. In some embodiments, X is substituted with 1, 2, or 3 sechexyl. In some embodiments, X is tertbutyl substituted with 1, 2, or 3 halogen atoms.

In some embodiments, X is methyl. In some embodiments, X is ethyl. In some embodiments, X is propyl. In some embodiments, X is butyl. In some embodiments, X is pentyl. In some embodiments, X is hexyl. In some embodiments, each X is isopropyl. In some embodiments, X is isobutyl. In some embodiments, X is isopentyl. In some embodiments, X is isohexyl. In some embodiments, X is secbutyl. In some embodiments, X is secpentyl. In some embodiments, X is sechexyl. In some embodiments, X is tertbutyl.

In some embodiments, each instance of alkyl substituted with 0, 1, 2, or 3 halogen atoms. In some embodiments, each instance of alk—is substituted with 0, 1, 2, or 3 halogen atoms.

### (iii) Additional Embodiments

In some embodiments, the compound is of Formula (II-a), (II-b), (II-c), (II-d), (II-e), (II-f), (II-g), (II-h), or (II-i):

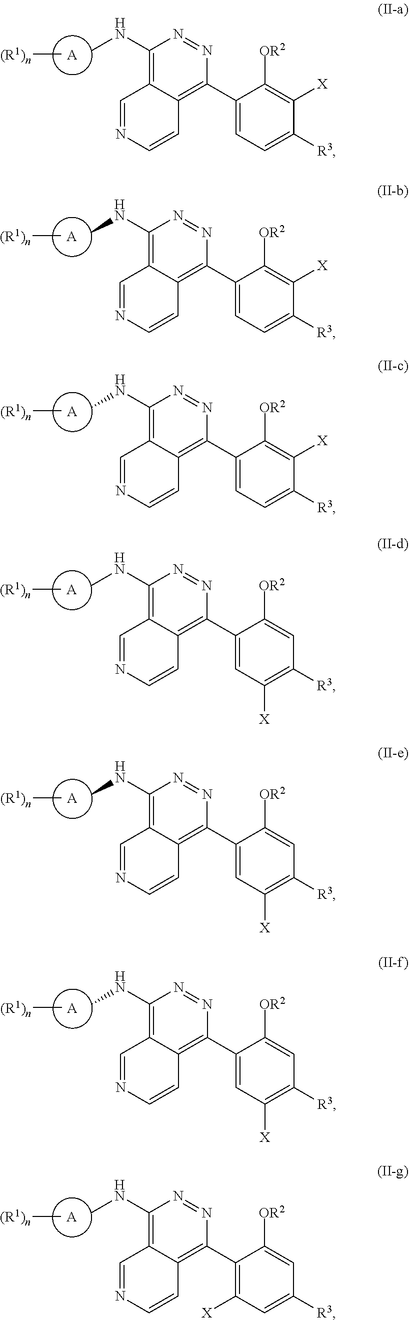

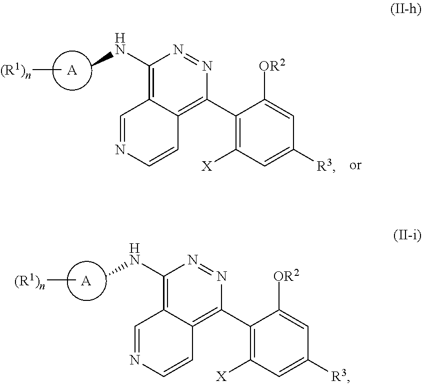

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments, R² is H; R³ is halogen; X is H or halogen; and n is 0. In some embodiments, R² is H; R³ is C₁-C₆ haloalkyl or C₁-C₆ alkyl; X is H; and n is 0. In some embodiments, R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments, R² is H; R³ is —CN; X is H or halogen; and n is 0. In some embodiments, R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. Formula (II-a), (II-b), and (II-c) exemplify subgenera where a non-hydrogen X is located at the ortho position relative to the —OR² group. Formula (II-g), (II-h), and (II-i) exemplify subgenera where a non-hydrogen X is located at the meta position relative to the —OR² group. Formula (II-d), (II-e), and (II-f) exemplify subgenera where a non-hydrogen X is located at the para position relative to the —OR² group. In some embodiments, X is an ortho fluoro group. In some embodiments, X is a meta fluoro group. In some embodiments, X is H.

In some embodiments, the compound is of Formula (III-a), (III-b), or (III-c):

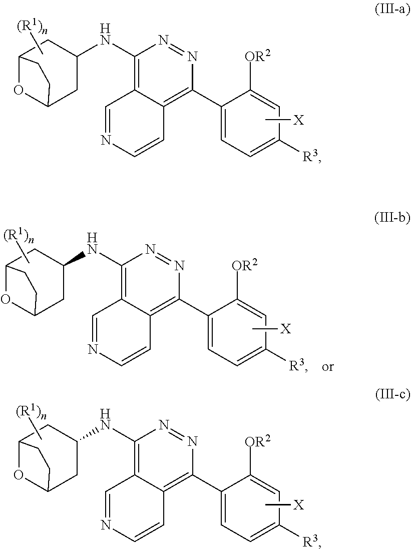

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments, R² is H. R³ is halogen; X is H or halogen; and n is 0. In some embodiments, R² is H; R³ is C₁-C₆ haloalkyl or C₁-C₆ alkyl; X is H; and n is 0. In some embodiments, R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments, R² is H; R³ is —CN; X is H or halogen; and n is 0. In some embodiments, R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. In some embodiments, when X is halogen or C₁-C₆ alkyl (e.g., CH₃), X is located at the ortho position relative to the —OR² group. In some embodiments, when X is halogen or C₁-C₆ alkyl (e.g., —CH₃), X is located at the meta position relative to the —OR² group. In some embodiments, X is an ortho fluoro group. In some embodiments, X is a meta fluoro group. In some embodiments, X is H.

In some embodiments, the compound is of Formula (III-a1), (III-b1), or (III-c1):

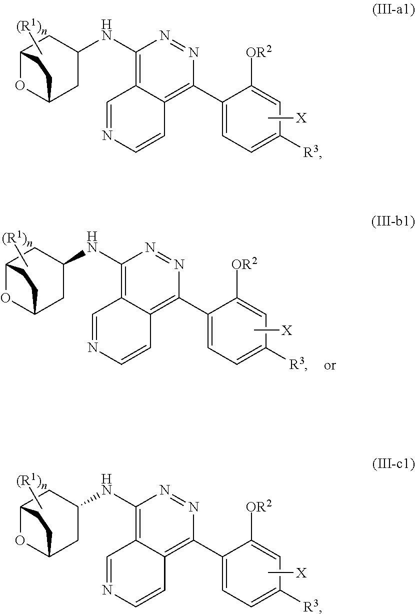

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments. R³ is H; R³ is halogen; X is H or halogen; and n is 0. In some embodiments, R² is H; R³ is C₁-C₆ haloalkyl or C₁-C₆ alkyl; X is H; and n is 0. In some embodiments. R² is H; R³ is C₁-C₆ haloalkyl or C₁-C₆ alkyl; X is halogen; and n is 0. In some embodiments. R² is H; R³ is C₁-C₆ haloalkyl or C₁-C₆ alkyl; X is fluoro; and n is 0. In some embodiments, R² is H; R³ is —CN; X is H or halogen; and n is 0. In some embodiments, R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. In some embodiments, when X is halogen or C₁-C₆ alkyl (e.g., —CH₃), X is located at the ortho position relative to the —OR² group. In some embodiments, when X is halogen or C₁-C₆ alkyl (e.g., CH₃), X is located at the meta position relative to the —OR group. In some embodiments, X is an ortho fluoro group. In some embodiments, X is a meta fluoro group. In some embodiments, X is H.

In some embodiments, the compound is of Formula (IV-a), (IV-b), or (IV-c):

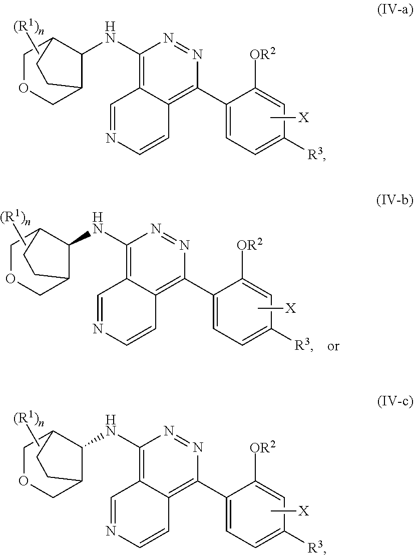

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments. R² is H; R³ is halogen; X is H or halogen; and n is 0. In some embodiments, R² is H; R³ is C₁-C₆ haloalkyl or C₁-C₆ alkyl; X is H; and n is 0. In some embodiments, R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments, R² is H; R³ is —CN; X is H or halogen; and n is 0. In some embodiments, R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. In some embodiments, when X is halogen or C₁-C₆ alkyl (e.g., CH₃), X is located at the ortho position relative to the —OR² group. In some embodiments, when X is halogen or C₁-C₆ alkyl (e.g., —CH₃), X is located at the meta position relative to the —OR² group. In some embodiments, X is an ortho fluoro group. In some embodiments, X is a meta fluoro group. In some embodiments, X is H.

In some embodiments, the compound is of Formula (IV-a1), (IV-b1), or (IV-c1):

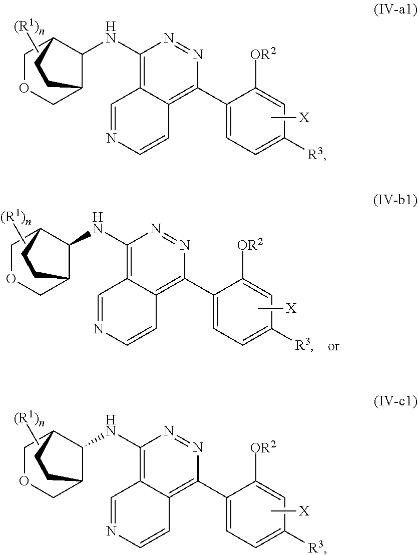

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments, R² is H; R³ is halogen; X is H or halogen; and n is 0. In some embodiments, R² is H; R³ is C₁-C₆ haloalkyl or C₁-C₆ alkyl; X is H; and n is 0. In some embodiments, R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments. R² is H; R³ is —CN; X is H or halogen; and n is 0. In some embodiments, R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. In some embodiments, when X is halogen or C₁-C₆ alkyl (e.g., CH₃), X is located at the ortho position relative to the —OR² group. In some embodiments, when X is halogen or C₁-C₆ alkyl (e.g., —CH₃), X is located at the meta position relative to the —OR² group. In some embodiments, X is an ortho fluoro group. In some embodiments, X is a meta fluoro group. In some embodiments, X is H.

In some embodiments, the compound is of Formula (V-a), (V-b), or (V-c):

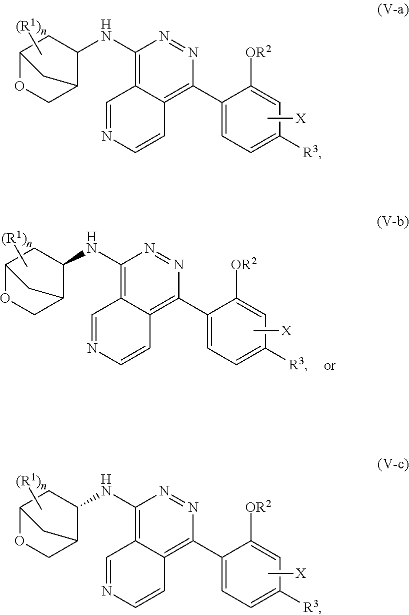

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments, R² is H; R³ is halogen; X is H or halogen; and n is 0. In some embodiments, R² is H; R³ is C₁-C₆ haloalkyl or C₁-C₆ alkyl; X is H; and n is 0. In some embodiments, R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments, X is H, halogen, or C₁₋₆ alkyl, wherein each instance of alkyl is independently substituted with 0, 1, 2, or 3 halogen atoms. In some embodiments, R² is H; R³ is —CN; X is H or halogen; and n is 0. In some embodiments, R² is H; R³ is —CN; X is H or fluoro; and n is 0. In some embodiments, when X is halogen or C₁-C₆ alkyl (e.g., —CH₃), X is located at the ortho position relative to the —OR² group. In some embodiments, when X is halogen or C₁-C₆ alkyl (e.g., —CH₃), X is located at the meta position relative to the —OR² group. In some embodiments, X is an ortho fluoro group. In some embodiments, X is a meta fluoro group. In some embodiments, X is H.

In some embodiments, the compound is of Formula (V-a1), (V-b1), (V-c1), (V-a2), (V-b2), or (V-c2):

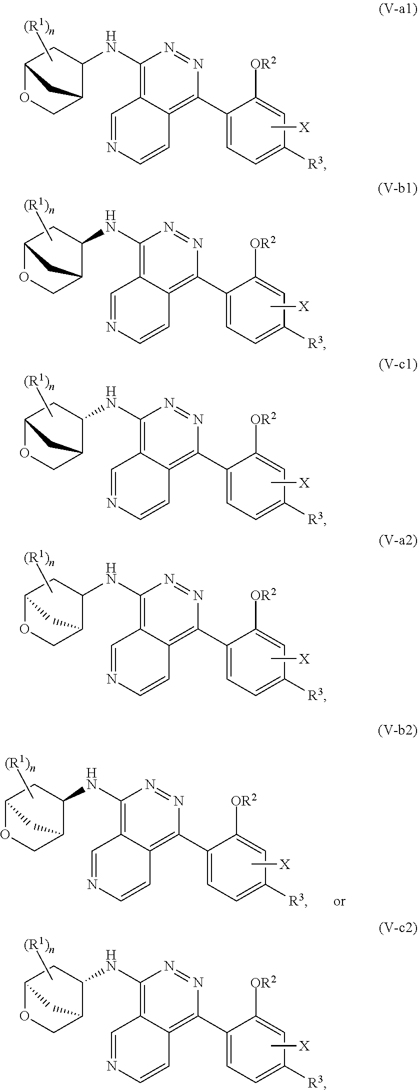

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments, R² is H; R³ is halogen; X is H or halogen; and n is 0. In some embodiments, R² is H; R³ is C₁-C₆ haloalkyl or C₁-C₆ alkyl; X is H; and n is 0. In some embodiments. R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments. R² is H; R³ is —CN; X is H or halogen; and n is 0. In some embodiments, R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. In some embodiments, when X is halogen or C₁-C₆ alkyl (e.g., CH₃), X is located at the ortho position relative to the —OR² group. In some embodiments, when X is halogen or C₁-C₆ alkyl (e.g., —CH₃), X is located at the meta position relative to the —OR² group. In some embodiments, X is an ortho fluoro group. In some embodiments, X is a meta fluoro group. In some embodiments, X is H.

In some embodiments, the compound is of Formula (VI-a), (VI-b), or (VI-c):

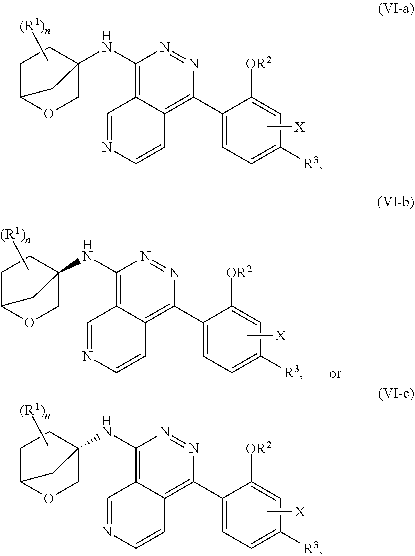

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments, R² is H; R³ is halogen; X is H or halogen; and n is 0. In some embodiments, R² is H; R³ is C₁-C₆ haloalkyl or C₁-C₆ alkyl; X is H; and n is 0. In some embodiments, R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments. R² is H; R³ is —CN; X is H or halogen; and n is 0. In some embodiments. R² is H; R³ is —CN; X is H or halogen; and n is 0. In some embodiments, R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. In some embodiments, when X is halogen or C₁-C₆ alkyl (e.g., —CH₃). X is located at the ortho position relative to the —OR² group. In some embodiments, when X is halogen or C₁-C₆ alkyl (e.g., —CH₃), X is located at the meta position relative to the —OR² group. In some embodiments, X is an ortho fluoro group. In some embodiments, X is a meta fluoro group. In some embodiments, X is H. However, in some embodiments, compounds of Formula (VI-a), (VI-b), or (VI-c) are specifically excluded.

In some embodiments, the compound is of Formula (VII-a), (VII-b), or (VII-c):

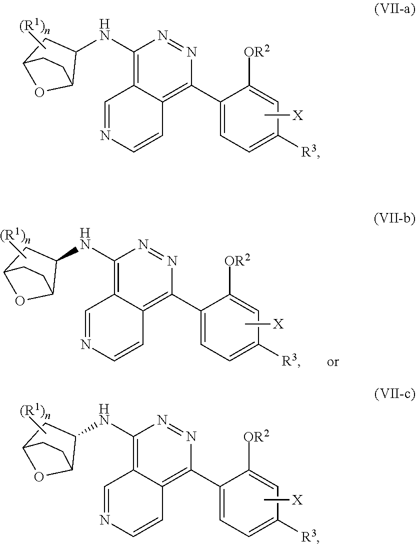

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments, R² is H; R³ is halogen; X is H or halogen; and n is 0. In some embodiments, R² is H; R³ is C₁-C₆ haloalkyl or C₁-C₆ alkyl; X is H; and n is 0. In some embodiments, R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments, R² is H; R³ is —CN; X is H or halogen; and n is 0. In some embodiments, R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. In some embodiments, when X is halogen or C₁-C₆ alkyl (e.g., CH₃), X is located at the ortho position relative to the —OR² group. In some embodiments, when X is halogen or C₁-C₆ alkyl (e.g., —CH₃), X is located at the meta position relative to the —OR² group. In some embodiments, X is an ortho fluoro group. In some embodiments, X is a meta fluoro group. In some embodiments, X is H.

In some embodiments, the compound is of Formula (VII-a1). (VII-b1), (VII-c1), (VII-a2), (VII-b2), or (VII-c2):

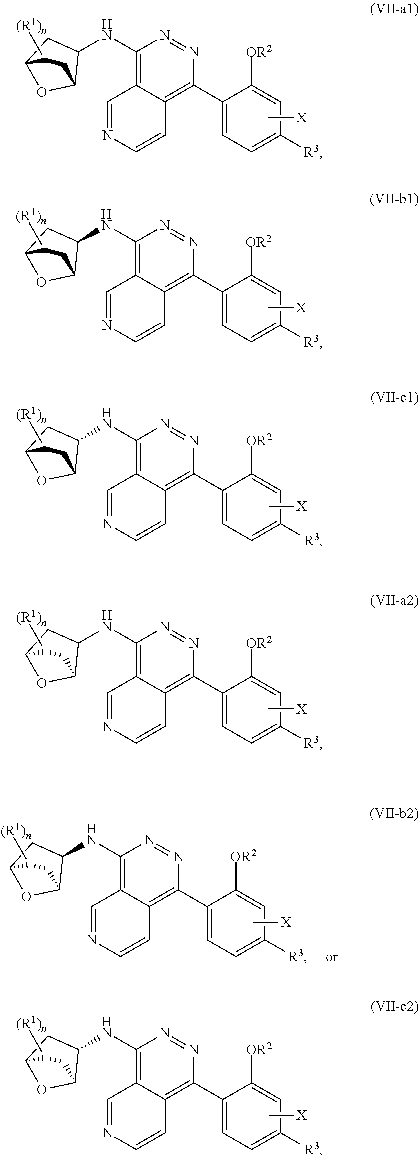

or a pharmaceutically acceptable salt or isotopically labeled derivative thereof. In some embodiments, R² is H; R³ is halogen, C₁-C₆ haloalkyl, or C₁-C₆ alkyl; X is H or halogen; and n is 0. In some embodiments, R² is H; R³ is C₁-C₆ haloalkyl or C₁-C₆ alkyl; X is H; and n is 0. In some embodiments, R² is H; R³ is C₁-C₆ haloalkyl or C₁-C₆ alkyl; X is halogen; and n is 0. In some embodiments, R² is H; R³ is C₁-C₆ haloalkyl or C₁-C₆ alkyl; X is fluoro; and n is 0. In some embodiments, R² is H; R³ is —CN; X is H or halogen; and n is 0. In some embodiments. R² is H; R³ is —CN; X is H or fluoro; and n is 0. In some embodiments, X is H, halogen, or C₁₋₆ alkyl, wherein each instance of alkyl is independently substituted with 0, 1, 2, or 3 halogen atoms. In some embodiments, when X is halogen or C₁-C₆ alkyl (e.g., CH₃), X is located at the ortho position relative to the —OR² group. In some embodiments, when X is halogen or C₁-C₆ alkyl (e.g., —CH₃), X is located at the meta position relative to the —OR² group. In some embodiments, X is an ortho fluoro group. In some embodiments, X is a meta fluoro group. In some embodiments, X is H.

In some embodiments, the compound is selected from a compound of Tables 1, 2 or 3, or a pharmaceutically acceptable salt or isotopically labeled derivative thereof.

In some embodiments, the compound is selected from a compound of Table 1, or a pharmaceutically acceptable salt or isotopically labeled derivative thereof.

In some embodiments, the compound is selected from a compound of Table 2, or a pharmaceutically acceptable salt or isotopically labeled derivative thereof.

In some embodiments, the compound is selected from a compound of Table 3, or a pharmaceutically acceptable salt or isotopically labeled derivative thereof.

If a stereochemical position is arbitrarily assigned, an Asterix (*) is included as part of the compound number. If a stereochemical position is rationally assigned, an Asterix and dashed “r” (*-r) is included as part of the compound number. If the absolute stereochemistry has been determined or is retroactively assigned based on that known stereochemistry, no Asterix or dashed “r” (*-r) is included. Rational assignment signifies there is a correlation between the designated assignment and a known absolute assignment. The Ex #signifies where the corresponding compound is described in a numbered Example, or in Table B or Table C of the Examples.

TABLE 1

| Ex# | Cmpd. # | Compound Structure/Name |
|---|---|---|
| Table B | 1 | 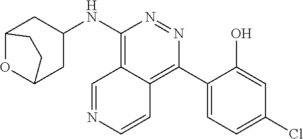 [SMILES: Oc1cc(Cl)ccc1-c1nnc(NC2CC3CCC(C2)O3)c2cnccc12] 2-(4-((8-oxabicyclo[3.2.1]octan-3-yl)amino)pyrido[3,4-d]pyridazin-1-yl)-5- chlorophenol |
| 1 | 1A*-r | 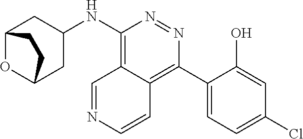 2-(4-(((1R,3r,5S)-8-oxabicyclo[3.2.1]octan-3-yl)amino)pyrido[3,4- d]pyridazin-1-yl)-5-chlorophenol |
| Table B | 1B*-r | 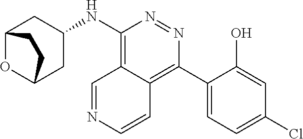 2-(4-(((1R,3s,5S)-8-oxabicyclo[3.2.1]octan-3-yl)amino)pyrido[3,4- d]pyridazin-1-yl)-5-chlorophenol |
| Table B | 2 | 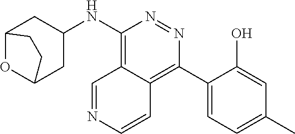 [SMILES: Cc1ccc(-c2nnc(NC3CC4CCC(C3)O4)c3cnccc23)c(O)c1] 2-(4-((8-oxabicyclo[3.2.1]octan-3-yl)amino)pyrido[3,4-d]pyridazin-1-yl)-5- methylphenol |
| 2 | 2A*-r | 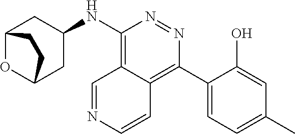 2-(4-(((1R,3r,5S)-8-oxabicyclo[3.2.1]octan-3-yl)amino)pyrido[3,4- d]pyridazin-1-yl)-5-methylphenol |
| 3 | 2B*-r | 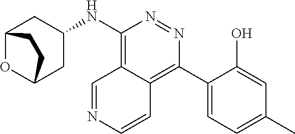 2-(4-(((1R,3s,5S)-8-oxabicyclo[3.2.1]octan-3-yl)amino)pyrido[3,4- d]pyridazin-1-yl)-5-methylphenol |
| 6 | 3 | 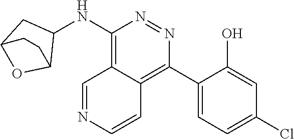 [SMILES: Oc1cc(Cl)ccc1-c1nnc(NC2CC3CCC2O3)c2cnccc12] 2-(4-((7-oxabicyclo[2.2.1]heptan-2-yl)amino)pyrido[3,4-d]pyridazin-1-yl)-5- chlorophenol |
| 6 | 3′* | 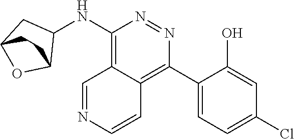 [SMILES: Oc1cc(Cl)ccc1-c1nnc(NC2C[C@@H]3CC[C@H]2O3)c2cnccc12] 2-(4-(((1R,4S)-7-oxabicyclo[2.2.1]heptan-2-yl)amino)pyrido[3,4-d]pyridazin- 1-yl)-5-chlorophenol |
| 6 | 3″* | 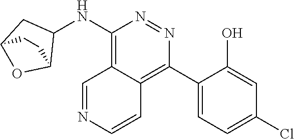 [SMILES: Oc1cc(Cl)ccc1-c1nnc(NC2C[C@H]3CC[C@@H]2O3)c2cnccc12] 2-(4-(((1S,4R)-7-oxabicyclo[2.2.1]heptan-2-yl)amino)pyrido[3,4-d]pyridazin- 1-yl)-5-chlorophenol |
| 6 | 3A* | 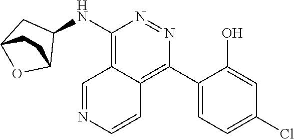 2-(4-(((1R,2R,4S)-7-oxabicyclo[2.2.1]heptan-2-yl)amino)pyrido[3,4- d]pyridazin-1-yl)-5-chlorophenol |
| 6 | 3B* | 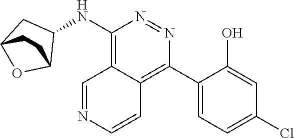 2-(4-(((1R,2S,4S)-7-oxabicyclo[2.2.1]heptan-2-yl)amino)pyrido[3,4- d]pyridazin-1-yl)-5-chlorophenol |
| 6 | 3C* | 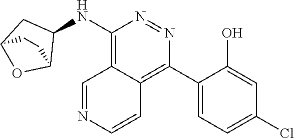 [SMILES: Oc1cc(Cl)ccc1-c1nnc(N[C@@H]2C[C@H]3CC[C@@H]2O3)c2cnccc12] 2-(4-(((1S,2R,4R)-7-oxabicyclo[2.2.1]heptan-2-yl)amino)pyrido[3,4- d]pyridazin-1-yl)-5-chlorophenol |
| 6 | 3D* | 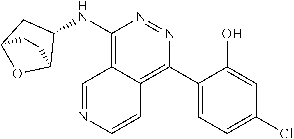 [SMILES: Oc1cc(Cl)ccc1-c1nnc(N[C@H]2C[C@H]3CC[C@@H]2O3)c2cnccc12] 2-(4-(((1S,2S,4R)-7-oxabicyclo[2.2.1]heptan-2-yl)amino)pyrido[3,4- d]pyridazin-1-yl)-5-chlorophenol |
| Table C | 4 | 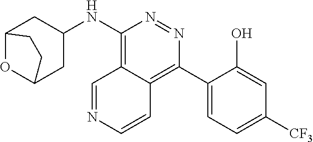 [SMILES: Oc1cc(C(F)(F)F)ccc1-c1nnc(NC2CC3CCC(C2)O3)c2cnccc12] 2-(4-((8-oxabicyclo[3.2.1]octan-3-yl)amino)pyrido[3,4-d]pyridazin-1-yl)-5- (trifluoromethyl)phenol |
| 7 | 4A*-r | 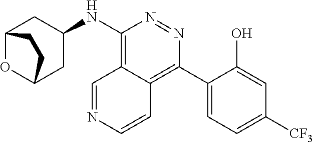 2-(4-(((1R,3r,5S)-8-oxabicyclo[3.2.1]octan-3-yl)amino)pyrido[3,4- d]pyridazin-1-yl)-5-(trifluoromethyl)phenol |
| Table C | 4B*-r | 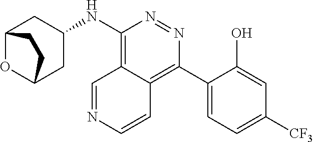 2-(4-(((1R,3s,5S)-8-oxabicyclo[3.2.1]octan-3-yl)amino)pyrido[3,4- d]pyridazin-1-yl)-5-(trifluoromethyl)phenol |
| Table B | 5 | 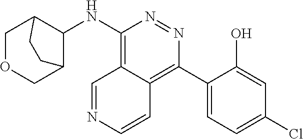 [SMILES: Oc1cc(Cl)ccc1-c1nnc(NC2C3CCC2COC3)c2cnccc12] 2-(4-((3-oxabicyclo[3.2.1]octan-8-yl)amino)pyrido[3,4-d]pyridazin-1-yl)-5- chlorophenol |
| 4 | 5A | 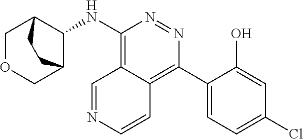 [SMILES: Oc1cc(Cl)ccc1-c1nnc(N[C@H]2[C@@H]3CC[C@H]2COC3)c2cnccc12] 2-(4-(((1R,5S,8s)-3-oxabicyclo[3.2.1]octan-8-yl)amino)pyrido[3,4- d]pyridazin-1-yl)-5-chlorophenol |
| Table B | 5B | 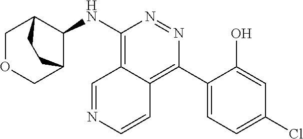 [SMILES: Oc1cc(Cl)ccc1-c1nnc(N[C@@H]2[C@@H]3CC[C@H]2COC3)c2cnccc12] 2-(4-(((1R,5S,8r)-3-oxabicyclo[3.2.1]octan-8-yl)amino)pyrido[3,4- d]pyridazin-1-yl)-5-chlorophenol |
| Table B | 6 | 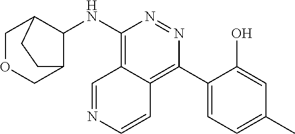 [SMILES: Cc1ccc(-c2nnc(NC3C4CCC3COC4)c3cnccc23)c(O)c1] 2-(4-((3-oxabicyclo[3.2.1]octan-8-yl)amino)pyrido[3,4-d]pyridazin-1-yl)-5- methylphenol |
| 5 | 6A | 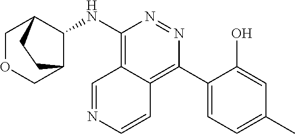 [SMILES: Cc1ccc(-c2nnc(N[C@H]3[C@@H]4CC[C@H]3COC4)c3cnccc23)c(O)c1] 2-(4-(((1R,5S,8s)-3-oxabicyclo[3.2.1]octan-8-yl)amino)pyrido[3,4- d]pyridazin-1-yl)-5-methylphenol |
| Table B | 6B | 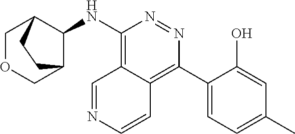 [SMILES: Cc1ccc(-c2nnc(N[C@@H]3[C@@H]4CC[C@H]3COC4)c3cnccc23)c(O)c1] 2-(4-(((1R,5S,8r)-3-oxabicyclo[3.2.1]octan-8-yl)amino)pyrido[3,4- d]pyridazin-1-yl)-5-methylphenol |
| Table B | 7 | 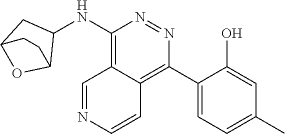 [SMILES: Cc1ccc(-c2nnc(NC3CC4CCC3O4)c3cnccc23)c(O)c1] 2-(4-((7-oxabicyclo[2.2.1]heptan-2-yl)amino)pyrido[3,4-d]pyridazin-1-yl)-5- methylphenol |
| Table B | 7′* | 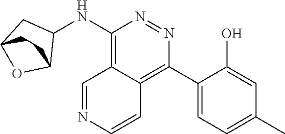 [SMILES: Cc1ccc(-c2nnc(NC3C[C@@H]4CC[C@H]3O4)c3cnccc23)c(O)c1] 2-(4-(((1R,4S)-7-oxabicyclo[2.2.1]heptan-2-yl)amino)pyrido[3,4-d]pyridazin- 1-yl)-5-methylphenol |
| Table B | 7″* | 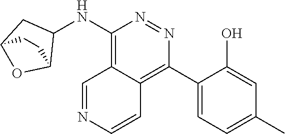 [SMILES: Cc1ccc(-c2nnc(NC3C[C@H]4CC[C@@H]3O4)c3cnccc23)c(O)c1] 2-(4-(((1S,4R)-7-oxabicyclo[2.2.1]heptan-2-yl)amino)pyrido[3,4-d]pyridazin- 1-yl)-5-methylphenol |
| Table B | 7A* | 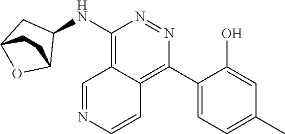 2-(4-(((1R,2R,4S)-7-oxabicyclo[2.2.1]heptan-2-yl)amino)pyrido[3,4- d]pyridazin-1-yl)-5-methylphenol |
| Table B | 7B* | 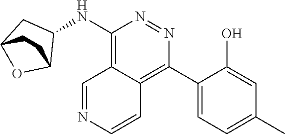 2-(4-(((1R,2S,4S)-7-oxabicyclo[2.2.1]heptan-2-yl)amino)pyrido[3,4- d]pyridazin-1-yl)-5-methylphenol |
| Table B | 7C* | 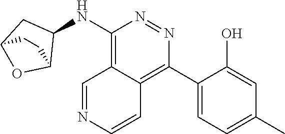 [SMILES: Cc1ccc(-c2nnc(N[C@@H]3C[C@H]4CC[C@@H]3O4)c3cnccc23)c(O)c1] 2-(4-(((1S,2R,4R)-7-oxabicyclo[2.2.1]heptan-2-yl)amino)pyrido[3,4- d]pyridazin-1-yl)-5-methylphenol |
| Table B | 7D* | 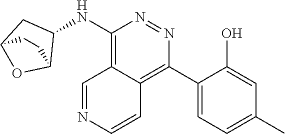 [SMILES: Cc1ccc(-c2nnc(N[C@H]3C[C@H]4CC[C@@H]3O4)c3cnccc23)c(O)c1] 2-(4-(((1S,2S,4R)-7-oxabicyclo[2.2.1]heptan-2-yl)amino)pyrido[3,4- d]pyridazin-1-yl)-5-methylphenol |
| Table B | 8 | 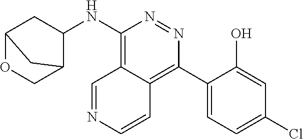 [SMILES: Oc1cc(Cl)ccc1-c1nnc(NC2CC3CC2CO3)c2cnccc12] 2-(4-((2-oxabicyclo[2.2.1]heptan-5-yl)amino)pyrido[3,4-d]pyridazin-1-yl)-5- chlorophenol |
| Table B | 8A* | 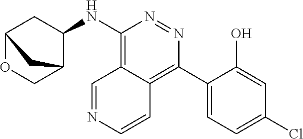 [SMILES: Oc1cc(Cl)ccc1-c1nnc(N[C@@H]2C[C@@H]3C[C@H]2CO3)c2cnccc12] 2-(4-(((1S,4R,5R)-2-oxabicyclo[2.2.1]heptan-5-yl)amino)pyrido[3,4- d]pyridazin-1-yl)-5-chlorophenol |
| Table B | 8B* | 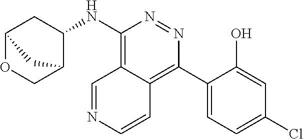 [SMILES: Oc1cc(Cl)ccc1-c1nnc(N[C@H]2C[C@H]3C[C@@H]2CO3)c2cnccc12] 2-(4-(((1R,4S,5S)-2-oxabicyclo[2.2.1]heptan-5-yl)amino)pyrido[3,4- d]pyridazin-1-yl)-5-chlorophenol |
| Table B | 9 | 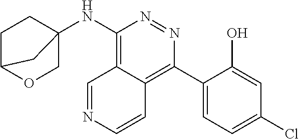 [SMILES: Oc1cc(Cl)ccc1-c1nnc(NC23CCC(C2)OC3)c2cnccc12] 2-(4-((2-oxabicyclo[2.2.1]heptan-4-yl)amino)pyrido[3,4-d]pyridazin-1-yl)-5- chlorophenol |
| Table B | 9A* | 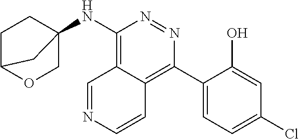 [SMILES: Oc1cc(Cl)ccc1-c1nnc(N[C@]23CCC(C2)OC3)c2cnccc12] 2-(4-(((4R)-2-oxabicyclo[2.2.1]heptan-4-yl)amino)pyrido[3,4-d]pyridazin-1- yl)-5-chlorophenol |
| Table B | 9B* | 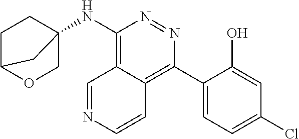 [SMILES: Oc1cc(Cl)ccc1-c1nnc(N[C@@]23CCC(C2)OC3)c2cnccc12] 2-(4-(((4S)-2-oxabicyclo[2.2.1]heptan-4-yl)amino)pyrido[3,4-d]pyridazin-1- yl)-5-chlorophenol |
| 12 | 10 | 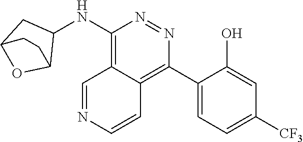 [SMILES: Oc1cc(C(F)(F)F)ccc1-c1nnc(NC2CC3CCC2O3)c2cnccc12] 2-(4-((7-oxabicyclo[2.2.1]heptan-2-yl)amino)pyrido[3,4-d]pyridazin-1-yl)-5- (trifluoromethyl)phenol |
| 12 | 10′* | 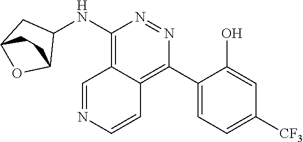 [SMILES: Oc1cc(C(F)(F)F)ccc1-c1nnc(NC2C[C@@H]3CC[C@H]2O3)c2cnccc12] 2-(4-(((1R,4S)-7-oxabicyclo[2.2.1]heptan-2-yl)amino)pyrido[3,4-d]pyridazin- 1-yl)-5-(trifluoromethyl)phenol |
| 12 | 10″* | 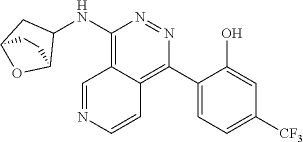 [SMILES: Oc1cc(C(F)(F)F)ccc1-c1nnc(NC2C[C@H]3CC[C@@H]2O3)c2cnccc12] 2-(4-(((1S,4R)-7-oxabicyclo[2.2.1]heptan-2-yl)amino)pyrido[3,4-d]pyridazin- 1-yl)-5-(trifluoromethyl)phenol |
| 12 | 10′″* | 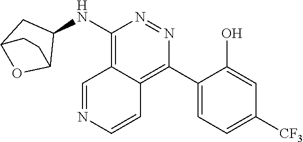 [SMILES: Oc1cc(C(F)(F)F)ccc1-c1nnc(N[C@@H]2CC3CCC2O3)c2cnccc12] 2-(4-(((2R)-7-oxabicyclo[2.2.1]heptan-2-yl)amino)pyrido[3,4-d]pyridazin-1- yl)-5-(trifluoromethyl)phenol |
| 12 | 10″″* | 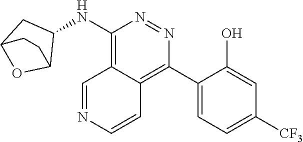 [SMILES: Oc1cc(C(F)(F)F)ccc1-c1nnc(N[C@H]2CC3CCC2O3)c2cnccc12] 2-(4-(((2S)-7-oxabicyclo[2.2.1]heptan-2-yl)amino)pyrido[3,4-d]pyridazin-1- yl)-5-(trifluoromethyl)phenol |
| 12 | 10A* | 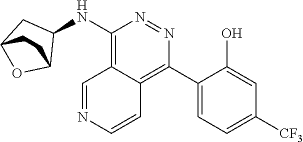 2-(4-(((1R,2R,4S)-7-oxabicyclo[2.2.1]heptan-2-yl)amino)pyrido[3,4- d]pyridazin-1-yl)-5-(trifluoromethyl)phenol |
| 12 | 10B* | 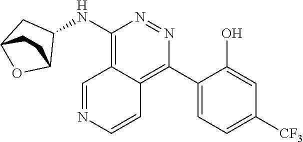 2-(4-(((1R,2S,4S)-7-oxabicyclo[2.2.1]heptan-2-yl)amino)pyrido[3,4- d]pyridazin-1-yl)-5-(trifluoromethyl)phenol |
| 12 | 10C* | 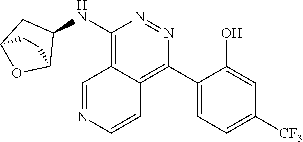 [SMILES: Oc1cc(C(F)(F)F)ccc1-c1nnc(N[C@@H]2C[C@H]3CC[C@@H]2O3)c2cnccc12] 2-(4-(((1S,2R,4R)-7-oxabicyclo[2.2.1]heptan-2-yl)amino)pyrido[3,4- d]pyridazin-1-yl)-5-(trifluoromethyl)phenol |
| 12 | 10D* | 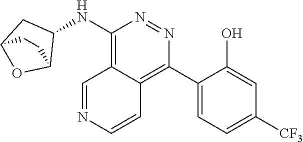 [SMILES: Oc1cc(C(F)(F)F)ccc1-c1nnc(N[C@H]2C[C@H]3CC[C@@H]2O3)c2cnccc12] 2-(4-(((1S,2S,4R)-7-oxabicyclo[2.2.1]heptan-2-yl)amino)pyrido[3,4- d]pyridazin-1-yl)-5-(trifluoromethyl)phenol |
| Table C | 11 | 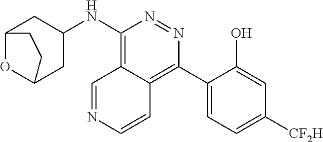 [SMILES: Oc1cc(C(F)F)ccc1-c1nnc(NC2CC3CCC(C2)O3)c2cnccc12] 2-(4-((8-oxabicyclo[3.2.1]octan-3-yl)amino)pyrido[3,4-d]pyridazin-1-yl)-5- (difluoromethyl)phenol |
| Table C | 11A*-r | 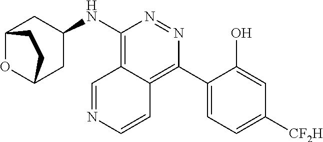 2-(4-(((1R,3r,5S)-8-oxabicyclo[3.2.1]octan-3-yl)amino)pyrido[3,4- d]pyridazin-1-yl)-5-(difluoromethyl)phenol |
| Table C | 11B*-r | 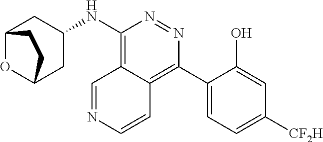 2-(4-(((1R,3s,5S)-8-oxabicyclo[3.2.1]octan-3-yl)amino)pyrido[3,4- d]pyridazin-1-yl)-5-(difluoromethyl)phenol |
| Table C | 12 | 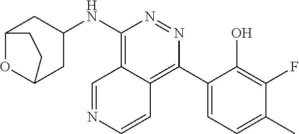 [SMILES: Cc1ccc(-c2nnc(NC3CC4CCC(C3)O4)c3cnccc23)c(O)c1F] 6-(4-((8-oxabicyclo[3.2.1]octan-3-yl)amino)pyrido[3,4-d]pyridazin-1-yl)-2- fluoro-3-methylphenol |
| Table C | 12A*-r | 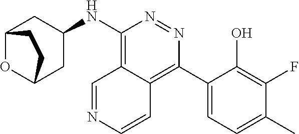 6-(4-(((1R,3r,5S)-8-oxabicyclo[3.2.1]octan-3-yl)amino)pyrido[3,4- d]pyridazin-1-yl)-2-fluoro-3-methylphenol |
| Table C | 12B*-r | 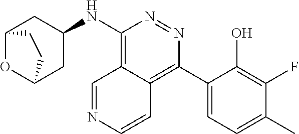 [SMILES: Cc1ccc(-c2nnc(N[C@@H]3C[C@H]4CC[C@@H](C3)O4)c3cnccc23)c(O)c1F] 6-(4-(((1R,3s,5S)-8-oxabicyclo[3.2.1]octan-3-yl)amino)pyrido[3,4- dipyridazin-1-yl)-2-fluoro-3-methylphenol |
| Table C | 13 | 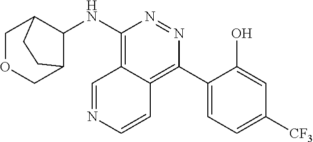 [SMILES: Oc1cc(C(F)(F)F)ccc1-c1nnc(NC2C3CCC2COC3)c2cnccc12] 2-(4-((3-oxabicyclo[3.2.1]octan-8-yl)amino)pyrido[3,4-d]pyridazin-1-yl)-5- (trifluoromethyl)phenol |
| Table C | 13A | 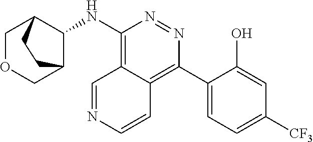 [SMILES: Oc1cc(C(F)(F)F)ccc1-c1nnc(N[C@H]2[C@@H]3CC[C@H]2COC3)c2cnccc12] 2-(4-(((1R,5S,8s)-3-oxabicyclo[3.2.1]octan-8-yl)amino)pyrido[3,4- d]pyridazin-1-yl)-5-(trifluoromethyl)phenol |
| Table C | 13B | 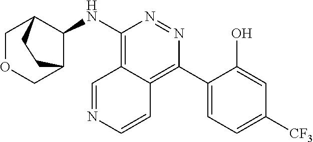 [SMILES: Oc1cc(C(F)(F)F)ccc1-c1nnc(N[C@@H]2[C@@H]3CC[C@H]2COC3)c2cnccc12] 2-(4-(((1R,5S,8r)-3-oxabicyclo[3.2.1]octan-8-yl)amino)pyrido[3,4- d]pyridazin-1-yl)-5-(trifluoromethyl)phenol |
| Table C | 14 | 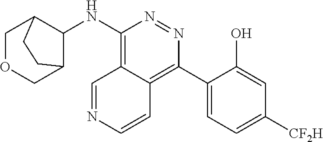 [SMILES: Oc1cc(C(F)F)ccc1-c1nnc(NC2C3CCC2COC3)c2cnccc12] 2-(4-((3-oxabicyclo[3.2.1]octan-8-yl)amino)pyrido[3,4-d]pyridazin-1-yl)-5- (difluoromethyl)phenol |
| Table C | 14A | 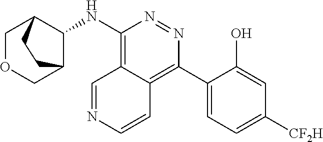 [SMILES: Oc1cc(C(F)F)ccc1-c1nnc(N[C@H]2[C@@H]3CC[C@H]2COC3)c2cnccc12] 2-(4-(((1R,5S,8s)-3-oxabicyclo[3.2.1]octan-8-yl)amino)pyrido[3,4- d]pyridazin-1-yl)-5-(difluoromethyl)phenol |
| Table C | 14B | 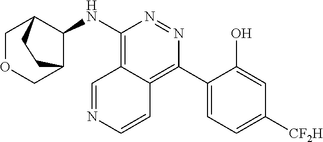 [SMILES: Oc1cc(C(F)F)ccc1-c1nnc(N[C@@H]2[C@@H]3CC[C@H]2COC3)c2cnccc12] 2-(4-(((1R,5S,8r)-3-oxabicyclo[3.2.1]octan-8-yl)amino)pyrido[3,4- d]pyridazin-1-yl)-5-(difluoromethyl)phenol |
| Table C | 15 | 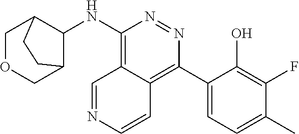 [SMILES: Cc1ccc(-c2nnc(NC3C4CCC3COC4)c3cnccc23)c(O)c1F] 6-(4-((3-oxabicyclo[3.2.1]octan-8-yl)amino)pyrido[3,4-d]pyridazin-1-yl)-2- fluoro-3-methylphenol |
| 8 | 15A | 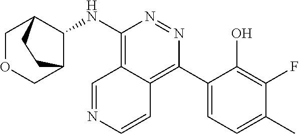 [SMILES: Cc1ccc(-c2nnc(N[C@H]3[C@@H]4CC[C@H]3COC4)c3cnccc23)c(O)c1F] 6-(4-(((1R,5S,8s)-3-oxabicyclo[3.2.1]octan-8-yl)amino)pyrido[3,4- d]pyridazin-1-yl)-2-fluoro-3-methylphenol |
| Table C | 15B | 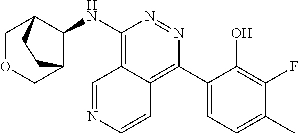 [SMILES: Cc1ccc(-c2nnc(N[C@@H]3[C@@H]4CC[C@H]3COC4)c3cnccc23)c(O)c1F] 6-(4-(((1R,5S,8r)-3-oxabicyclo[3.2.1]octan-8-yl)amino)pyrido[3,4- d]pyridazin-1-yl)-2-fluoro-3-methylphenol |

TABLE 2

| Ex# | Cmpd. # | Compound Structure/Name |
|---|---|---|
| Table C | 16 | 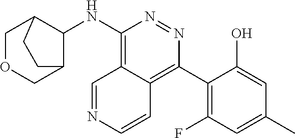 [SMILES: Cc1cc(O)c(-c2nnc(NC3C4CCC3COC4)c3cnccc23)c(F)c1] 2-(4-((3-oxabicyclo[3.2.1]octan-8-yl)amino)pyrido[3,4-d]pyridazin-1-yl)-3- fluoro-5-methylphenol |
| 9 | 16A | 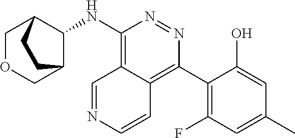 2-(4-(((1R,5S,8s)-3-oxabicyclo[3.2.1]octan-8-yl)amino)pyrido[3,4- d]pyridazin-1-yl)-3-fluoro-5-methylphenol |
| Table C | 16B | 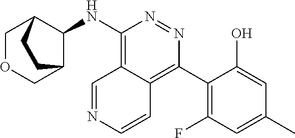 2-(4-(((1R,5S,8r)-3-oxabicyclo[3.2.1]octan-8-yl)amino)pyrido[3,4- d]pyridazin-1-yl)-3-fluoro-5-methylphenol |
| Table C | 17 | 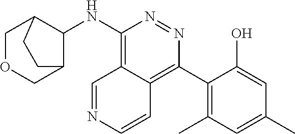 [SMILES: Cc1cc(C)c(-c2nnc(NC3C4CCC3COC4)c3cnccc23)c(O)c1] 2-(4-((3-oxabicyclo[3.2.1]octan-8-yl)amino)pyrido[3,4-d]pyridazin-1-yl)-3,5- dimethylphenol |
| 10 | 17A | 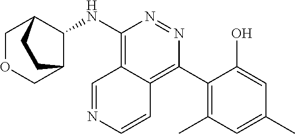 2-(4-(((1R,5S,8s)-3-oxabicyclo[3.2.1]octan-8-yl)amino)pyrido[3,4- d]pyridazin-1-yl)-3,5-dimethylphenol |
| Table C | 17B | 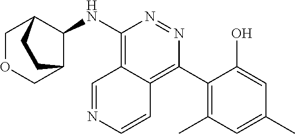 2-(4-(((1R,5S,8r)-3-oxabicyclo[3.2.1]octan-8-yl)amino)pyrido[3,4- d]pyridazin-1-yl)-3,5-dimethylphenol |

TABLE 3

| Ex# | Cmpd. # | Compound Structure/Name |
|---|---|---|
| Table C | 18 | 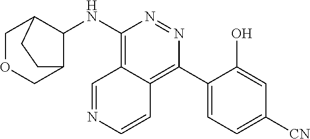 [SMILES: N#Cc1ccc(-c2nnc(NC3C4CCC3COC4)c3cnccc23)c(O)c1] 4-(4-((3-oxabicyclo[3.2.1]octan-8-yl)amino)pyrido[3,4-d]pyridazin-1-yl)-3- hydroxybenzonitrile |
| 11 | 18A | 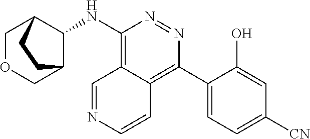 4-(4-(((1R,5S,8s)-3-oxabicyclo[3.2.1]octan-8-yl)amino)pyrido[3,4- d]pyridazin-1-yl)-3-hydroxybenzonitrile |
| Table C | 18B | 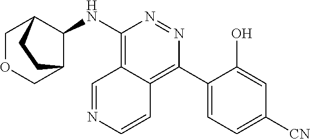 4-(4-(((1R,5S,8r)-3-oxabicyclo[3.2.1]octan-8-yl)amino)pyrido[3,4- d]pyridazin-1-yl)-3-hydroxybenzonitrile |

### (iii) Biological Assays

Compounds designed, selected and/or optimized by methods described above, once produced, can be characterized using a variety of assays known to those skilled in the art to determine whether the compounds have biological activity. For example, the compounds can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, binding assays, cellular assays (cell lines, primary cells and whole blood), in vitro cell viability assays, as well as assays for determining NLRP3 potency, unbound clearance, solubility, permeability, metabolic stability (e.g., in hepatocytes), and CYP inhibition and time-dependent inhibition (TDI) assays (e.g., for de-risking potential adverse in vivo drug-drug interactions).

Inhibitory Activity and Potency. In some embodiments, the biological assay is described in the Examples, Assay Methods section. For example, in some embodiments, the compounds of the instant disclosure may be tested for their human NLRP3 inhibition activity using known procedures, such as the methodology reported in Coll et al. Nat Med. (2015) 21 (3): 248-255. In some embodiments, the compounds of the instant disclosure may be tested for their human NLRP3 potency using known procedures. See, e.g., the human whole blood NLRP3 assay described in the Assay Methods section of the Examples.

Brain Penetrance. In some embodiments, the compounds of the instant disclosure may further be tested for brain penetrance. See. e.g., the Kp and Kpu,u NLRP3 assay described in the Assay Methods section of the Examples. As used herein, a Kpu,u value >0.3 calculated as provided in the Examples is considered brain penetrant, and a Kpu,u value <0.3 is not considered brain penetrant. As noted in the Assay Method, if the Kpu,u value is not determined, the Kp value may be useful as a metric of potential brain penetrance if the Kp value is >0.3.

In some embodiments, the compound has a Kpu,u of >0.3 to about 10.

In some embodiments, the compound has a Kpu,u of >0.3 to about 9. In some embodiments, the compound has a Kpu,u of >0.3 to about 8. In some embodiments, the compound has a Kpu,u of >0.3 to about 7. In some embodiments, the compound has a Kpu,u of >0.3 to about 6. In some embodiments, the compound has a Kpu,u of >0.3 to about 5. In some embodiments, the compound has a Kpu,u of >0.3 to about 4. In some embodiments, the compound has a Kpu,u of >0.3 to about 3. In some embodiments, the compound has a Kpu,u of >0.3 to about 2. In some embodiments, the compound has a Kpu,u of >0.3 to about 1.

In some embodiments, the compound has a Kpu,u of about 0.3. In some embodiments, the compound has a Kpu,u of about 0.4. In some embodiments, the compound has a Kpu,u of about 0.5.

In some embodiments, the compound has a Kpu,u of about 1. In some embodiments, the compound has a Kpu,u of about 1.5.

In some embodiments, the compound has a Kpu,u of about 2. In some embodiments, the compound has a Kpu,u of about 2.5.

In some embodiments, the compound has a Kpu,u of about 3. In some embodiments, the compound has a Kpu,u of about 3.5.

In some embodiments, the compound has a Kpu,u of about 4. In some embodiments, the compound has a Kpu,u of about 4.5.

In some embodiments, the compound has a Kpu,u of about 5. In some embodiments, the compound has a Kpu,u of about 5.5.

In some embodiments, the compound has a Kpu,u of about 6. In some embodiments, the compound has a Kpu,u of about 6.5.

In some embodiments, the compound has a Kpu,u of about 7. In some embodiments, the compound has a Kpu,u of about 7.5.

In some embodiments, the compound has a Kpu,u of about 8. In some embodiments, the compound has a Kpu,u of about 8.5.

In some embodiments, the compound has a Kpu,u of about 9. In some embodiments, the compound has a Kpu,u of about 9.5.

In some embodiments, the compound has a Kpu,u of about 10.

In some embodiments, the compound has a Kpu,u of ≤0.3. In some embodiments, the compound has a Kpu,u of about 0.1 to ≤0.3. In some embodiments, the compound has a Kpu,u of about 0.2 to ≤0.3.

Stability. In some embodiments, the stability of compounds may be determined using a hepatocyte stability assay, which is used to determine the metabolic stability of a compound in hepatocytes (liver cells) or liver microsomes. This type of assay provides valuable information about how quickly a drug is metabolized in the liver and can be used to assess its potential effectiveness and safety in drug discovery. In one exemplary assay, hepatocytes from the species of interest (e.g., mouse, rat, dog, monkey, human) are incubated with the test compound at a controlled temperature of 37° C., for different time periods (e.g., 5, 15, 30, 60, and 120 minutes). At each time point during the incubation, samples are taken, the reaction is terminated, and the amount of test compound remaining analyzed using LC-MS/MS to monitor the disappearance of the test compound over time (Gradient). From these data, a half-life can be calculated (t ½=time it takes for ½ of the test compound to be consumed in the hepatocyte incubation). See, e.g., Coe et al., Methods in Pharmacology & Toxicology (2008) 151. In some embodiments, the compound is metabolically stable, e.g., having a half-life in mouse, rat, dog, human, or monkey liver microsomes or hepatocytes of >20 minutes, >30 minutes, >40 minutes, >50 minutes, >60 minutes, >120 minutes, >240 minutes, >480 minutes, between about 30 minutes to about 120 minutes, between about 60 minutes to about 120 minutes, or between about 60 minutes to about 480 minutes. Metabolic stability as expressed by half-life in mouse, rat, dog, human, or monkey liver microsomes or hepatocytes may be indicative of improved metabolic stability in human. See also Examples, Assay Methods, Mouse and Human Hepatocyte Stability Assays.

Solubility. In some embodiments, the solubility of compounds may be determined following known procedures, such as described in Alsenz and Kansy, Advanced Drug Delivery Reviews (2007) 59:546-567, and Wang et al. J Mass Spectrom. (2000) 35:71-76. For example, the kinetic solubility in physiologically relevant media, such as phosphate buffered solution (PBS, pH 7.4) or simulated gastric fluid (SGF), may be measured using serial dilution and two hour incubation period, followed by filtration, and reported in μM by LC-MS/MS. Thermodynamic solubility in physiologically relevant media may be measured by LC-MS/MS, after a twenty-four hour incubation, followed by filtration, and reported in mg/mL. Optimized solubility may be beneficial for manufacturing and further processing of the compound. Furthermore, optimized solubility allows for a more efficient in vitro analysis of the compound, including data collection around the compound's safety, drug-drug interactions, potency, selectivity, metabolism and permeability. See also Examples, Assay Methods, Solubility Protocol in Phosphate Buffered Saline (PBS). In general, a solubility of >20 mM in PBS, such as >100 mM, in PBS, may be a desirable solubility profile.

Clearance. In some embodiments, the clearance of compounds may be determined using a clearance assay. For example, mouse clearance may be measured by dosing C57BL6 mice via IV Bolus dose administration of 0.5 mg/kg of test compound formulized in 5% DMSO+10% Kolliphor HS-15, with blood being drawn at different timepoints. Concentration of test compound in blood at various timepoints may be quantified using LC-MS/MS. The clearance in mL/min/kg may be determined by dividing the dose administrated by the AUC (area under the curve-Blood conc vs time). See, e.g., Smith et al., Clearance in Drug Design (2019) 62:2245-2255. In some embodiments, the compounds may be tested for unbound clearance (Clu) following known procedures, such as described in Miller et al., J. Med. Chem. (2020) 63:12156-12170. For example, unbound clearance (Clu) may be calculated by dividing total clearance (CL″ in mL/min/kg) as measured in blood or plasma by the unbound fraction in plasma (fu).

Permeability and Efflux. In some embodiments, the permeability of compounds may be determined following known procedures, such as described in Wang et al. J Mass Spectrom. (2000) 35:71-76. For example, permeability across cell membranes may be measured using either Caco-2 or MDCK-MDR1 cell lines in Transwell plates, after measuring the compound in both apical and basolateral chambers, and reported as an apparent permeability Papp A-B in 10⁻⁶ cm/s. In some embodiments, the permeability of compounds may be determined using a MDCK-MDR1 permeability assay. This assay is a commonly used in vitro method to evaluate the permeability and efflux of compounds across cell monolayers. It specifically assesses the ability of a substance to be transported by the multidrug resistance protein 1 (MDR1), also known as P-glycoprotein (P-gp), which is an efflux transporter involved in the elimination of many drugs from cells. To perform the MDCK-MDR1 permeability assay, a cell line derived from Madin-Darby Canine Kidney (MDCK) cells that express the MDR1 protein is used. These modified MDCK cells form a monolayer on a permeable support, such as a Transwell® insert. The assay can be conducted by applying the test compound separately to both the apical side and basolateral side of the MDCK-MDR1 monolayer and incubating the cells at an appropriate temperature, typically 37° C., for a specific time period (2 hours in our experiment) to allow the compound to permeate through the monolayers. At the end of the incubations, samples are collected from both the apical and basolateral compartments and the concentration of the test compound in each compartment is determined using LC-MS/MS and a flux from apical to basolateral (A-B) direction and from basolateral to apical (B-A) direction are reported as apparent permeability's Papp in 10⁻⁶ cm/s. The efflux ratio, which represents the transport efficiency of the compound, is calculated by dividing the flux from basolateral to apical (Papp B-A) by the flux from apical to basolateral (Papp A-B). Sec, e.g., E. H.; Di. L.; Kerns, E. H. Drug-like properties: Concepts, Structure Design and methods; Academic Press, 2008.

hERG inhibition. The human ether-à-go-go related gene (hERG) is associated with cardiac potassium channel inhibition leading to QT-interval prolongation, a severe cardiovascular toxicity responsible for numerous drug attrition in the clinic, and low hERG inhibition decreases the risk of cardiovascular toxicity. A generally acceptable ranking system used to identify the potency of a test compound inhibiting hERG channel is as follows: a) Low: IC₅₀≥30 μM; b) Moderate: 10 μM<IC₅₀<30 μM; c) High: IC₅₀<10 μM. An exemplary assay which may be used to evaluate the potential inhibitory effect of a test compound on the hERG channel is a manual patch-clamp system performed using a transfected HEK293 cell line with a hERG gene, and using dofetilide as a positive control. See, e.g., Roche et al., ChemBioChem. (2002) 3:455-459; Glenn et al., Journal of Pharmacological and Toxicological Methods (2004) 50:93-101; and Roger et al., Computer Methods and Programs in Biomedicine (2004) 74, 167-181.

### (iv) Pharmaceutical Compositions

In some aspects, provided is a pharmaceutical composition comprising a compound of the present disclosure as an active ingredient, or a pharmaceutically acceptable salt or isotopically labeled derivative thereof, and one or more pharmaceutically acceptable excipients Exemplary pharmaceutically acceptable excipients include but are not limited carriers, fillers, vehicles, solubility enhancing agents, chelating agents, preservatives, tonicity agents, viscosity/suspending agents, buffers, pH modifying agents, and combinations thereof.

The compounds of the present disclosure may be formulated for oral administration in forms such as tablets, capsules (each of which includes sustained release or timed release formulations), pills, powders, granules, elixirs, tinctures, suspensions, syrups and emulsions. The compounds of the present disclosure may also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one other pharmaceutically acceptable excipient.

### (v) Methods of Use and Treatment

In some aspects, provided is a method of treating a disease or disorder disclosed herein in a subject in need thereof, comprising administering to the subject a therapeutically effective amount of a compound of the present disclosure or a pharmaceutically acceptable salt or isotopically labeled derivative thereof, or a pharmaceutical composition of the present disclosure.

In some embodiments, the disease or disorder is a disease or disorder in which NLRP3 activity is implicated.

In some aspects, the present disclosure provides a method of modulating NLRP3 activity (e.g., in vitro or in vivo), comprising contacting a cell with a compound of the present disclosure or a pharmaceutically acceptable salt or isotopically labeled derivative thereof.

In some aspects, the present disclosure provides a method of inhibiting NLRP3 activity (e.g., in vitro or in vivo), comprising contacting a cell with a compound of the present disclosure or a pharmaceutically acceptable salt or isotopically labeled derivative thereof.

In some embodiments, the disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an NLRP3-related disease in a subject that has been determined to carry a germline or somatic non-silent mutation in NLRP3.

In some embodiments, the disease or disorder is inflammation.

In some embodiments, the disease or disorder is an auto-immune disease.

In some embodiments, the disease or disorder is a cancer.

In some embodiments, the disease or disorder is an infection.

In some embodiments, the disease or disorder is a disease or disorder of the central nervous system.

In some embodiments, the disease or disorder is a metabolic disease.

In some embodiments, the disease or disorder is a cardiovascular disease.

In some embodiments, the disease or disorder is a respiratory disease.

In some embodiments, the disease or disorder is a kidney disease.

In some embodiments, the disease or disorder is a liver disease.

In some embodiments, the disease or disorder is an ocular disease.

In some embodiments, the disease or disorder is a skin disease.

In some embodiments, the disease or disorder is a lymphatic disease.

In some embodiments, the disease or disorder is a rheumatic disease.

In some embodiments, the disease or disorder is a psychological disease.

In some embodiments, the disease or disorder is graft versus host disease.

In some embodiments, the disease or disorder is allodynia.

In some embodiments, the disease or disorder is an NLRP3-related disease.

In some embodiments, the disease or disorder of the central nervous system is Parkinson's disease, Alzheimer's disease, traumatic brain injury, spinal cord injury, amyotrophic lateral sclerosis, or multiple sclerosis.

In some embodiments, the respiratory disease is steroid-resistant asthma.

In some embodiments, the respiratory disease is severe steroid-resistant asthma.

In some embodiments, the kidney disease is an acute kidney disease, a chronic kidney disease, or a rare kidney disease.

In some embodiments, the skin disease is psoriasis, hidradenitis suppurativa (HS), or atopic dermatitis.

In some embodiments, the rheumatic disease is dermatomyositis, Still's disease, or juvenile idiopathic arthritis.

In some embodiments, the NLRP3-related disease in a subject that has been determined to carry a germline or somatic non-silent mutation in NLRP3 is cryopyrin-associated autoinflammatory syndrome.

In some embodiments, the cryopyrin-associated autoinflammatory syndrome is familial cold autoinflammatory syndrome, Muckle-Wells syndrome, or neonatal onset multisystem inflammatory disease.

### (vi) Routes of Administration

A compound of the present disclosure, or a pharmaceutically acceptable salt or isotopically labeled derivative thereof, may be administered alone as a sole therapy or can be administered in addition with one or more other substances and/or treatments. Such combination treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering a compound of the instant disclosure with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which inflammasome activity is implicated comprising a compound of the disclosure as defined hereinbefore or a pharmaceutically acceptable salt or isotopically labeled derivative thereof, and another suitable agent.

According to a further aspect of the disclosure there is provided a pharmaceutical composition which comprises a compound of the disclosure, or a pharmaceutically acceptable salt or isotopically labeled derivative thereof, in combination with a suitable agent, in association with a pharmaceutically acceptable diluent or carrier.

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### (vii) Methods of Preparation

Compounds of Formula (I) may be synthesized following Synthetic Protocol A or Synthetic Protocol B, as provided below. The Examples further described non-limiting examples of the general syntheses.

For example, as depicted in Synthetic Protocol A, step one involves opening commercially available 3,4-pyridinedicarboxylic acid anhydride vii with a Grignard reagent of formula xx to obtain carboxylic acid viii. Step two features chlorination, then condensation with hydrazine to furnish pyridazinol ix. Step three then involves another chlorination to furnish key intermediate x, which in turn may be engaged in step four in SNAr reaction with an amine (i) to form an azaphthalazine xi. Step five then features optional alkyl ether (i.e., methyl ether) deprotection to provides analog xii as a compound of Formula (I). Each of the intermediates may exist as free bases or salts.

### Synthetic Protocol A

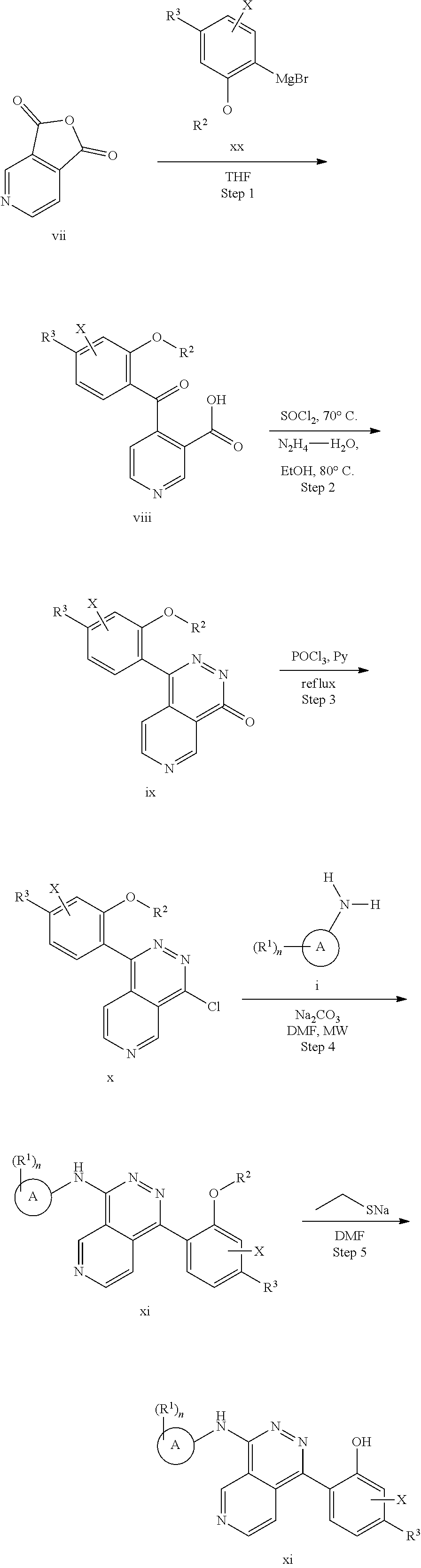

As depicted in Synthetic Protocol B, step one involves an SyAr reaction between an amine (i) and an heteroaryl dichloride (ii), to provide the target chloroaryl intermediate (iii). Step two involves cross-coupling between intermediate (iii), which may comprise a mixture of major and minor regioisomers, and a boronic acid or boronate (iv), where R′ is H or C₁₋₆ alkyl or two R′ groups are joined via a C₂-C₃ alkylene linker optionally substituted with one or more C₁₋₃ alkyl or C₁₋₃ haloalkyl, followed by optional deprotection in step 3 if R² is a not hydrogen (e.g., an alkyl ether such as a methyl ether), to generate the desired compound (v), which is a compound of Formula (I). Amine (i), aryl dichloride (ii), and boronic acid or boronate (iv) are commercially available or known in the chemical literature, unless otherwise indicated. Each of the intermediates may exist as free bases or salts.

### Synthetic Protocol B

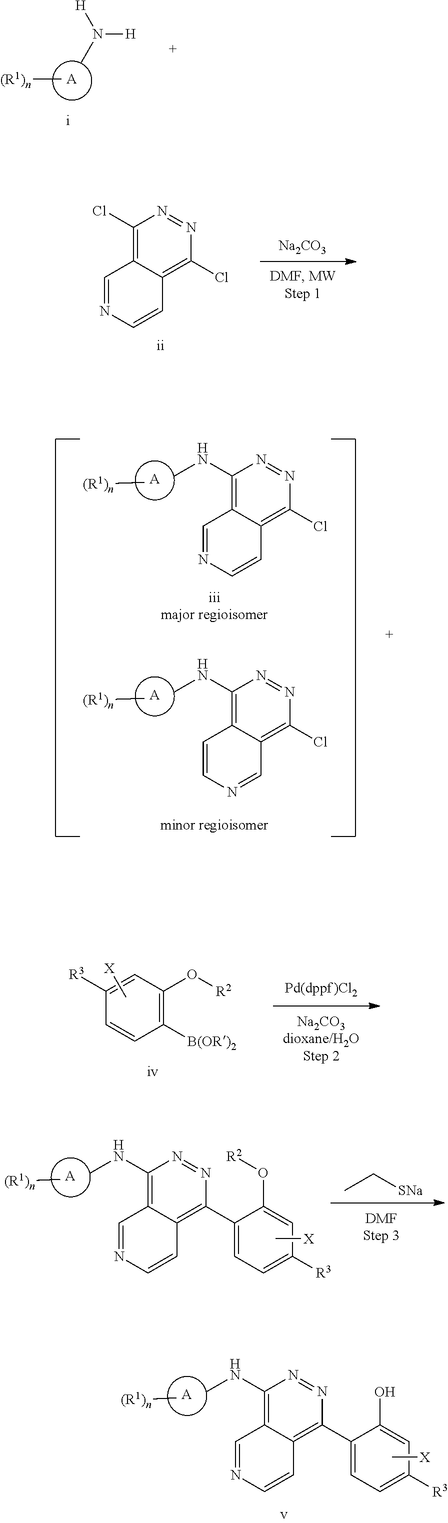

### (viii) Additional Embodiments

Exemplary Embodiment 1. A compound of Formula (I):

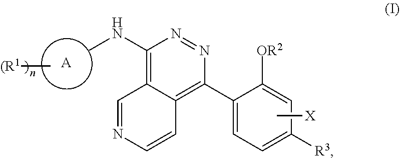

or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen, C₁-C₆ alkyl, or C₁-C₆ alkoxy;
R² is H, C₁-C₆ alkyl, or —C(O)(C₁-C₆ alkyl);
R³ is —OH, halogen, C₁-C₆ alkyl, or C₁-C₆ alkoxy;
X is H, —OH, halogen, —NH₂, —NH(C₁-C₆ alkyl), —N(C₁-C₆ alkyl) 2, or C₁-C₆ alkyl; and
n is 0, 1, 2, 3, or 4;
wherein each instance of alkyl or alk—is independently substituted with 0, 1, 2, or 3 halogen atoms.

Exemplary Embodiment 2. The compound of Exemplary Embodiment 1, or a pharmaceutically acceptable salt or isotopically labeled derivative thereof, wherein A is a 6-membered bridged bicyclic heterocycloalkyl comprising one O ring atom.

Exemplary Embodiment 3. The compound of Exemplary Embodiment 1, or a pharmaceutically acceptable salt or isotopically labeled derivative thereof, wherein A is a 7-membered bridged bicyclic heterocycloalkyl comprising one O ring atom.

Exemplary Embodiment 4. The compound of Exemplary Embodiment 1, or a pharmaceutically acceptable salt or isotopically labeled derivative thereof, wherein A is a 8-membered bridged bicyclic heterocycloalkyl comprising one O ring atom.

Exemplary Embodiment 5. The compound of any one of the preceding Exemplary Embodiments, or a pharmaceutically acceptable salt or isotopically labeled derivative thereof, wherein R² is H.

Exemplary Embodiment 6. The compound of any one of the preceding Exemplary Embodiments, or a pharmaceutically acceptable salt or isotopically labeled derivative thereof, wherein X is H or F.

Exemplary Embodiment 7. The compound of any one of the preceding Exemplary Embodiments, or a pharmaceutically acceptable salt or isotopically labeled derivative thereof, wherein n is 0.

Exemplary Embodiment 8. The compound of Exemplary Embodiment 1, or a pharmaceutically acceptable salt or isotopically labeled derivative thereof, wherein:
A is a 6- to 8-membered bridged bicyclic heterocycloalkyl, wherein the heterocycloalkyl comprises at least one O ring atom;
R² is H;
R³ is halogen. C₁-C₆ haloalkyl, or C₁-C₆ alkyl;
X is H or halogen; and
n is 0.

Exemplary Embodiment 9. The compound of any one of the preceding Exemplary Embodiments, or a pharmaceutically acceptable salt or isotopically labeled derivative thereof, wherein R³ is Cl, —CF₃, —CF₂H, or methyl.

Exemplary Embodiment 10. The compound of any one Exemplary Embodiments 1-8, or a pharmaceutically acceptable salt or isotopically labeled derivative thereof, wherein R³ is C₁-C₆ haloalkyl or C₁-C₆ alkyl.

Exemplary Embodiment 11. The compound of any one of the preceding Exemplary Embodiments, or a pharmaceutically acceptable salt or isotopically labeled derivative thereof, wherein R³ is —CF₃, —CF₂H, or methyl.

Exemplary Embodiment 12. The compound of any one of the preceding Exemplary Embodiments, wherein the compound is of Formula (II-a), (II-b), or (II-c):

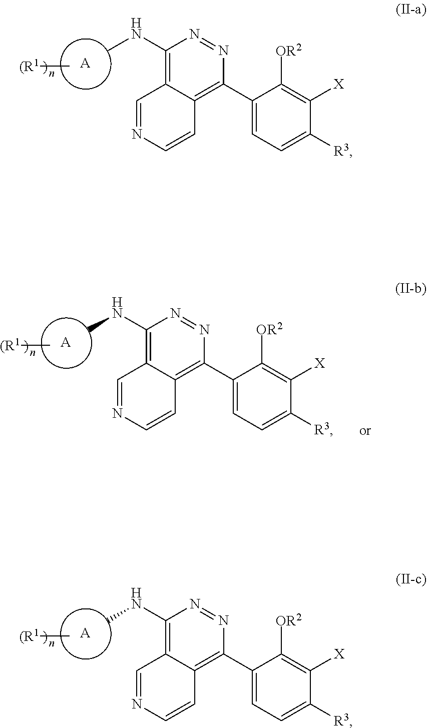

or a pharmaceutically acceptable salt or isotopically labeled derivative thereof.

Exemplary Embodiment 13. The compound of any one of the preceding Exemplary Embodiments, wherein the compound is of Formula (II-a), (II-b), (II-c), (II-d), (II-e), (II-f), (II-g), (II-h), or (II-i):

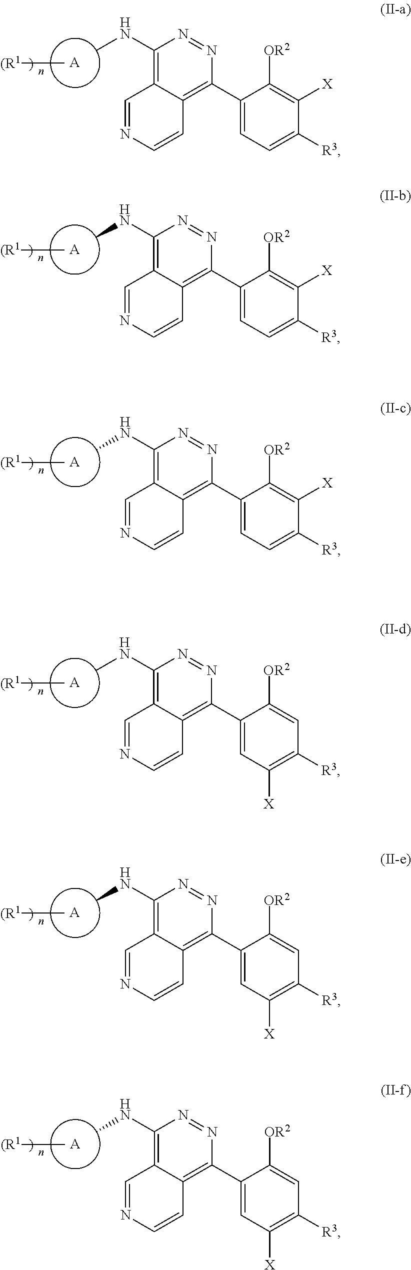

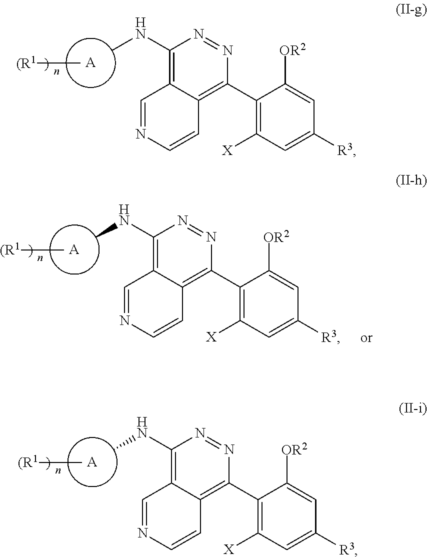

or a pharmaceutically acceptable salt or isotopically labeled derivative thereof.

Exemplary Embodiment 14. The compound of any one of the preceding Exemplary Embodiments, wherein the compound is of Formula (III-a), (III-b), or (III-c):

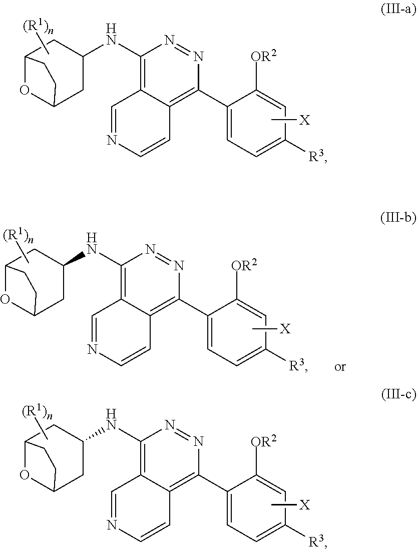

or a pharmaceutically acceptable salt or isotopically labeled derivative thereof.

Exemplary Embodiment 15. The compound of any one of the preceding Exemplary Embodiments, wherein the compound is of Formula (III-a1), (III-b1), or (III-c1):

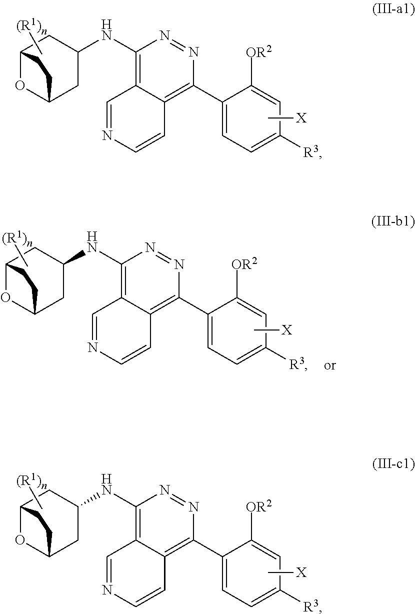

or a pharmaceutically acceptable salt or isotopically labeled derivative thereof.

Exemplary Embodiment 16. The compound of any one of Exemplary Embodiments 1-13, wherein the compound is of Formula (IV-a), (IV-b), or (IV-c):

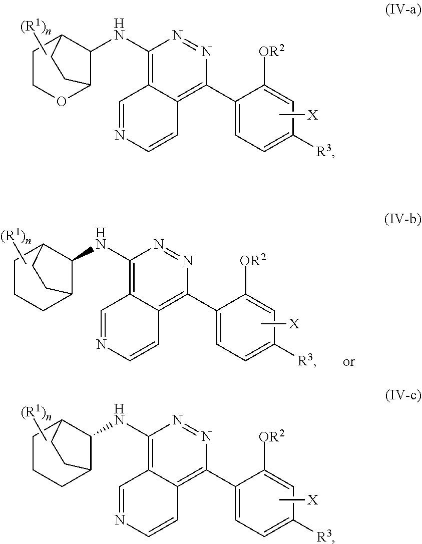

or a pharmaceutically acceptable salt or isotopically labeled derivative thereof.

Exemplary Embodiment 17. The compound of any one of Exemplary Embodiments 1-13, wherein the compound is of Formula (IV-a1), (IV-b1), or (IV-c1):

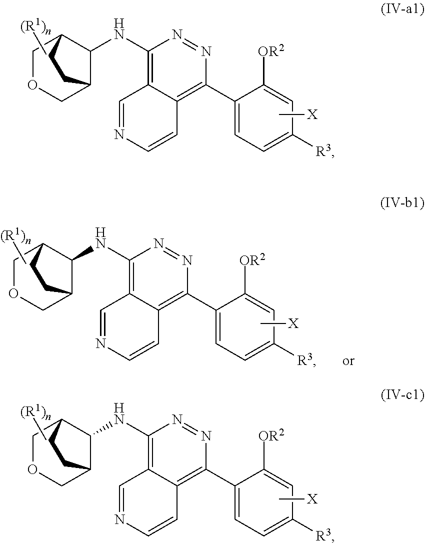

or a pharmaceutically acceptable salt or isotopically labeled derivative thereof.

Exemplary Embodiment 18. The compound of any one of Exemplary Embodiments 1-13, wherein the compound is of Formula (V-a), (V-b), or (V-c):

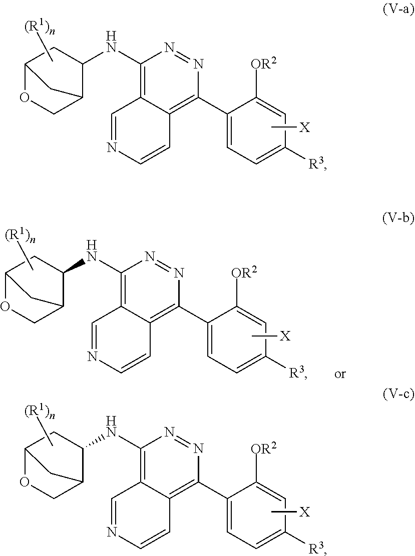

or a pharmaceutically acceptable salt or isotopically labeled derivative thereof.

Exemplary Embodiment 19. The compound of any one of Exemplary Embodiments 1-13, wherein the compound is of Formula (V-a1), (V-b1), (V-c1), (V-a2), (V-b2), or (V-c2):

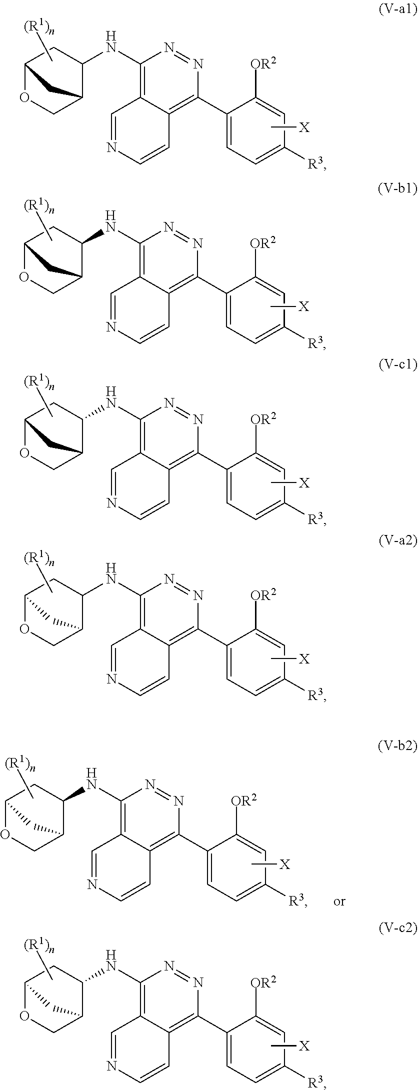

or a pharmaceutically acceptable salt or isotopically labeled derivative thereof.

Exemplary Embodiment 20. The compound of any one of Exemplary Embodiments 1-13, wherein the compound is of Formula (VI-a), (VI-b), or (VI-c):

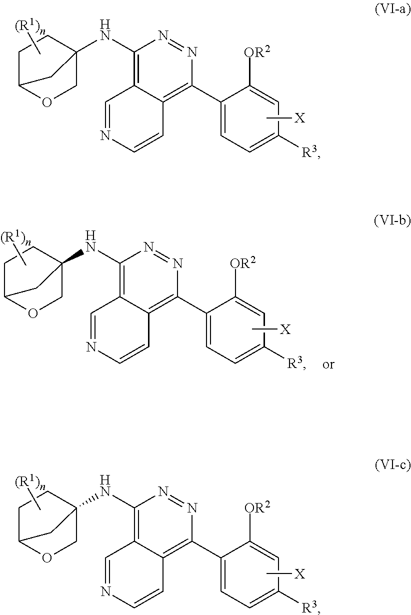

or a pharmaceutically acceptable salt or isotopically labeled derivative thereof.

Exemplary Embodiment 21. The compound of any one of Exemplary Embodiments 1-13, wherein the compound is of Formula (VII-a), (VII-b), or (VII-c):

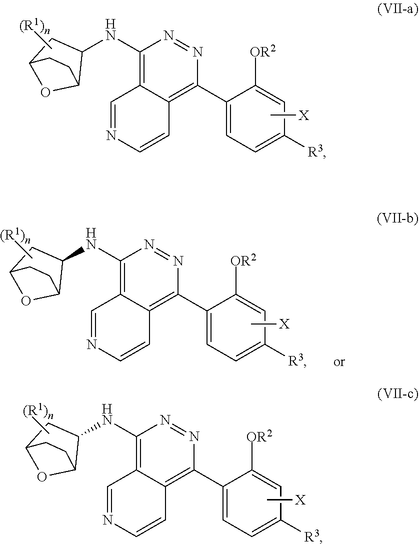

or a pharmaceutically acceptable salt or isotopically labeled derivative thereof.

Exemplary Embodiment 22. The compound of any one of Exemplary Embodiments 1-13, wherein the compound is of Formula (VII-a1), (VII-b1), (VII-c1), (VII-a2), (VII-b2), or (VII-c2):

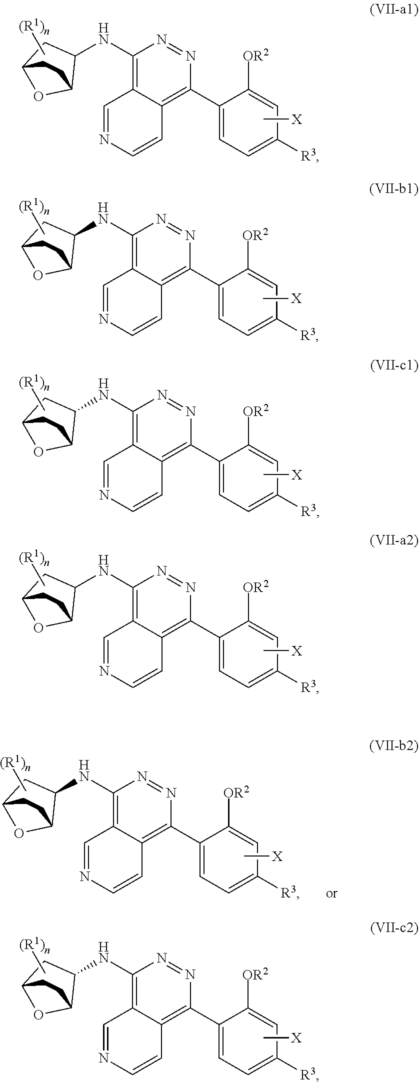

or a pharmaceutically acceptable salt or isotopically labeled derivative thereof.

Exemplary Embodiment 23. The compound of any one of Exemplary Embodiments 14-22, wherein X is halogen or C₁₋₆ alkyl independently substituted with 0, 1, 2, or 3 halogen atoms, further wherein X is located at the ortho or meta position relative to the —OR² group.

Exemplary Embodiment 24. The compound of Exemplary Embodiment 1 selected from a compound of Table 1 or Table 2, or a pharmaceutically acceptable salt thereof or isotopically labeled derivative thereof.

Exemplary Embodiment 25. The compound of any one of the preceding Exemplary Embodiments, or a pharmaceutically acceptable salt or isotopically labeled derivative thereof, wherein the compound has a Kpu,u>0.3.

Exemplary Embodiment 26. The compound of any one of the preceding Exemplary Embodiments, or a pharmaceutically acceptable salt or isotopically labeled derivative thereof, wherein the compound has a Kpu,u>0.3 to about 10.

Exemplary Embodiment 27. The compound of any one of Exemplary Embodiments 1-25, or a pharmaceutically acceptable salt or isotopically labeled derivative thereof, wherein the compound has a Kpu,u≤0.3.

Exemplary Embodiment 28. A pharmaceutical composition comprising the compound of any one of the preceding Exemplary Embodiments, or a pharmaceutically acceptable salt or isotopically labeled derivative thereof, and one or more pharmaceutically acceptable carriers.

Exemplary Embodiment 29. A method of modulating NLRP3, the method comprising administering to the subject a compound of any one of Exemplary Embodiments 1-27, or a pharmaceutically acceptable salt or isotopically labeled derivative thereof, or a pharmaceutical composition of Exemplary Embodiment 28.

Exemplary Embodiment 30. A method of treating a disease or disorder, the method comprising administering to the subject a compound of any one of Exemplary Embodiments 1-27, or a pharmaceutically acceptable salt or isotopically labeled derivative thereof, or a pharmaceutical composition of Exemplary Embodiment 28.

Exemplary Embodiment 31. The compound of any one of Exemplary Embodiments 1-27, or a pharmaceutically acceptable salt or isotopically labeled derivative thereof, or a pharmaceutical composition of Exemplary Embodiment 28, for use in treating a disease or disorder.

Exemplary Embodiment 32. Use of the compound of any one of Exemplary Embodiments 1-27, or a pharmaceutically acceptable salt or isotopically labeled derivative thereof, in the manufacture of a medicament, for the treatment of a disease or disorder.

Exemplary Embodiment 33. Use of the compound of any one of Exemplary Embodiments 1-27, or a pharmaceutically acceptable salt or isotopically labeled derivative thereof, for the treatment of a disease or disorder.

Exemplary Embodiment 34. The method, compound, or use of any one of Exemplary Embodiments 29-33, wherein the disease or disorder is an NLRP3-related disease or disorder.

Exemplary Embodiment 35. The method, compound, or use of any one of Exemplary Embodiments 29-34, wherein the subject is a human.

Exemplary Embodiment 36. The method, compound, or use of any one of Exemplary Embodiments 29-35, wherein the disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an NLRP3-related disease.

Exemplary Embodiment 37. The method, compound, or use of Exemplary Embodiment 36, wherein the disease or disorder of the central nervous system is Parkinson's disease, Alzheimer's disease, traumatic brain injury, spinal cord injury, amyotrophic lateral sclerosis, or multiple sclerosis.

Exemplary Embodiment 38. The method, compound, or use of Exemplary Embodiment 36, wherein the kidney disease is an acute kidney disease, a chronic kidney disease, or a rare kidney disease.

Exemplary Embodiment 39. The method, compound, or use of Exemplary Embodiment 36, wherein the skin disease is psoriasis, hidradenitis suppurativa (HS), or atopic dermatitis.

Exemplary Embodiment 40. The method, compound, or use of Exemplary Embodiment 36, wherein the rheumatic disease is dermatomyositis. Still's disease, or juvenile idiopathic arthritis.

Exemplary Embodiment 41. The method, compound, or use of Exemplary Embodiment 36, wherein the NLRP3-related disease is in a subject that has been determined to carry a germline or somatic non-silent mutation in NLRP3.

Exemplary Embodiment 42. The method, compound, or use of Exemplary Embodiment 41, wherein the NLRP3-related disease is in a subject that has been determined to carry a germline or somatic non-silent mutation in NLRP3 is cryopyrin-associated autoinflammatory syndrome.

Exemplary Embodiment 43. The method, compound, or use of Exemplary Embodiment 43, wherein the cryopyrin-associated autoinflammatory syndrome is familial cold autoinflammatory syndrome, Muckle-Wells syndrome, or neonatal onset multisystem inflammatory disease.

Exemplary Embodiment 1A. A compound of Formula (I):

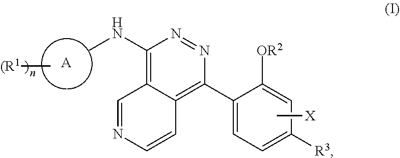

or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen, C₁-C₆ alkyl, or C₁-C₆ alkoxy;
R² is H, C₁-C₆ alkyl, or —C(O)(C₁-C₆ alkyl);
R³ is —OH, halogen, —CN, C₁-C₆ alkyl, or C₁-C₆ alkoxy;
X is H, —OH, halogen, —NH₂, —NH(C₁-C₆ alkyl), —N(C₁-C₆ alkyl) 2, or C₁-C₆ alkyl; and
n is 0, 1, 2, 3, or 4;
wherein each instance of alkyl or alk—is independently and optionally substituted with one or more halogen atoms.

Exemplary Embodiment 2A. The compound of Exemplary Embodiment 1A, or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen. C₁-C₆ alkyl, or C₁-C₆ alkoxy;
R² is H, C₁-C₆ alkyl, or —C(O)(C₁-C₆ alkyl);
R³ is —OH, halogen, C₁-C₆ alkyl, or C₁-C₆ alkoxy;
X is H, —OH, halogen, —NH₂, —NH(C₁-C₆ alkyl), —N(C₁-C₆ alkyl) 2, or C₁-C₆ alkyl; and
n is 0, 1, 2, 3, or 4;
wherein each instance of alkyl or alk—is independently substituted with 0, 1, 2, or 3 halogen atoms.

Exemplary Embodiment 3A. The compound of Exemplary Embodiment 1A, or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen, C₁-C₆ alkyl, or C₁-C₆ alkoxy;
R² is H, C₁-C₆ alkyl, or —C(O)(C₁-C₆ alkyl);
R³ is —CN;
X is H, —OH, halogen, —NH₂, —NH(C₁-C₆ alkyl), —N(C₁-C₆ alkyl)₂, or C₁-C₆ alkyl; and
n is 0, 1, 2, 3, or 4;
wherein each instance of alkyl or alk—is independently substituted with 0, 1, 2, or 3 halogen atoms.

Exemplary Embodiment 4A. The compound of any one of Exemplary Embodiments 1A-3A, or a pharmaceutically acceptable salt or isotopically labeled derivative thereof, wherein A is a 6-membered bridged bicyclic heterocycloalkyl comprising one O ring atom.

Exemplary Embodiment 5A. The compound of any one of Exemplary Embodiments 1A-3A, or a pharmaceutically acceptable salt or isotopically labeled derivative thereof, wherein A is a 7-membered bridged bicyclic heterocycloalkyl comprising one O ring atom.

Exemplary Embodiment 6A. The compound of any one of Exemplary Embodiments 1A-3A, or a pharmaceutically acceptable salt or isotopically labeled derivative thereof, wherein A is an 8-membered bridged bicyclic heterocycloalkyl comprising one O ring atom.

Exemplary Embodiment 7A. The compound of any one of the preceding Exemplary Embodiments, or a pharmaceutically acceptable salt or isotopically labeled derivative thereof, wherein R² is H.

Exemplary Embodiment 8A. The compound of any one of the preceding Exemplary Embodiments, or a pharmaceutically acceptable salt or isotopically labeled derivative thereof, wherein X is H or F.

Exemplary Embodiment 9A. The compound of any one of the preceding Exemplary Embodiments, or a pharmaceutically acceptable salt or isotopically labeled derivative thereof, wherein n is 0.

Exemplary Embodiment 10A. The compound of Exemplary Embodiment 1A, or a pharmaceutically acceptable salt or isotopically labeled derivative thereof, wherein:

A is a 6- to 8-membered bridged bicyclic heterocycloalkyl, wherein the heterocycloalkyl comprises at least one O ring atom:
R² is H;
R³ is halogen, C₁-C₆ haloalkyl, or C₁-C₆ alkyl;
X is H or halogen; and
n is 0.

Exemplary Embodiment 11A. The compound of any one of the preceding Exemplary Embodiments, or a pharmaceutically acceptable salt or isotopically labeled derivative thereof, wherein R³ is Cl, —CF₃, —CF H, or —CH₃.

Exemplary Embodiment 12A. The compound of any one Exemplary Embodiments 1A-10A, or a pharmaceutically acceptable salt or isotopically labeled derivative thereof, wherein R³ is C₁-C₆ haloalkyl or C₁-C₆ alkyl.

Exemplary Embodiment 13A. The compound of Exemplary Embodiment 12A, or a pharmaceutically acceptable salt or isotopically labeled derivative thereof, wherein R³ is —CF₃, —CF₂H, or —CH₃.

Exemplary Embodiment 14A. The compound of Exemplary Embodiment 1A, 3A, or 4A-9A, or a pharmaceutically acceptable salt or isotopically labeled derivative thereof, wherein:
A is a 6- to 8-membered bridged bicyclic heterocycloalkyl, wherein the heterocycloalkyl comprises at least one O ring atom;
R² is H;
R³ is —CN;
X is H or halogen; and
n is 0.

Exemplary Embodiment 15A. The compound of any one of Exemplary Embodiments 1A-14A, wherein the compound is of Formula (II-a), (II-b), (II-c), (II-d), (II-e), (II-f), (II-g), (II-h), or (II-i):

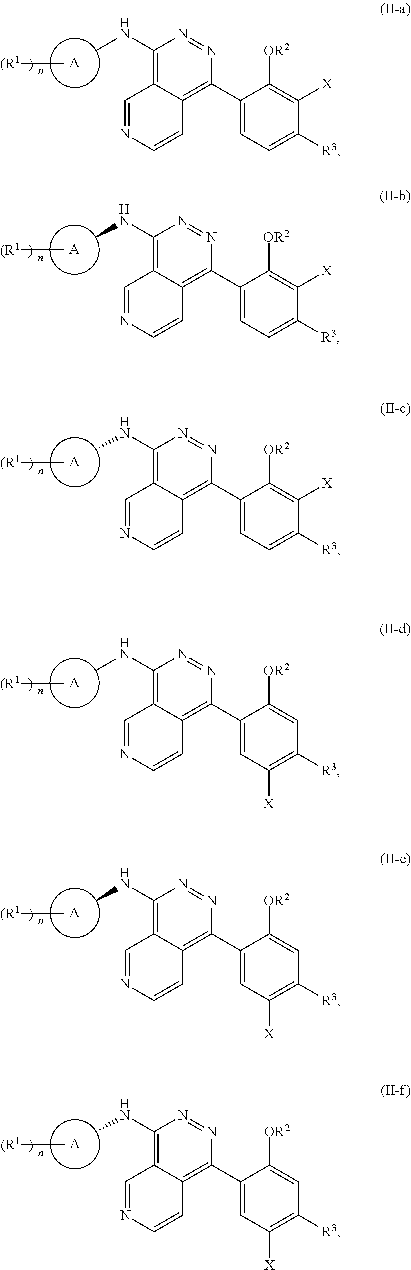

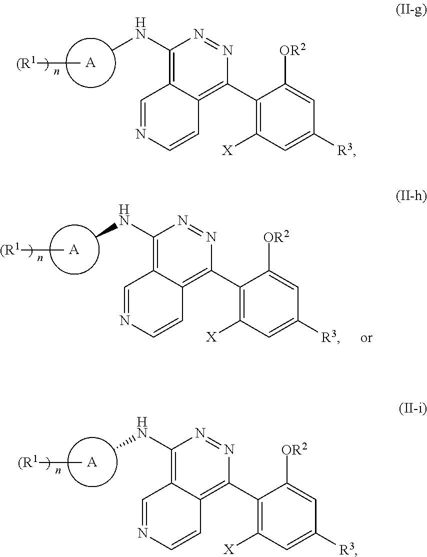

or a pharmaceutically acceptable salt or isotopically labeled derivative thereof.

Exemplary Embodiment 16A. The compound of any one of Exemplary Embodiments 1A-15A, wherein the compound is of Formula (III-a), (III-b), or (III-c):

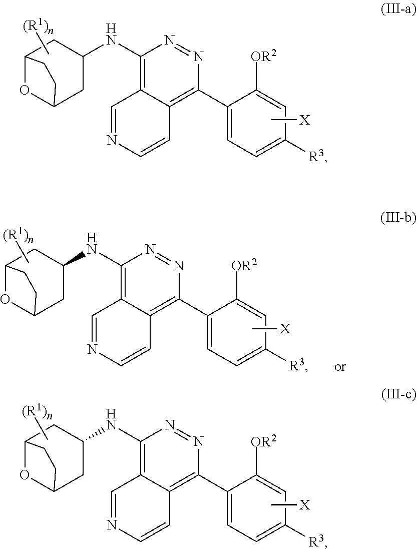

or a pharmaceutically acceptable salt or isotopically labeled derivative thereof.

Exemplary Embodiment 17A. The compound of any one of Exemplary Embodiments 1A-16A, wherein the compound is of Formula (III-a1), (III-b1), or (III-c1):

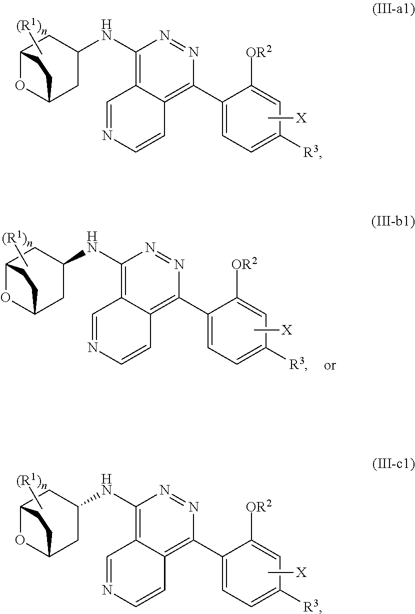

or a pharmaceutically acceptable salt or isotopically labeled derivative thereof.

Exemplary Embodiment 18A. The compound of any one of Exemplary Embodiments 1A-15A, wherein the compound is of Formula (IV-a), (IV-b), or (IV-c):

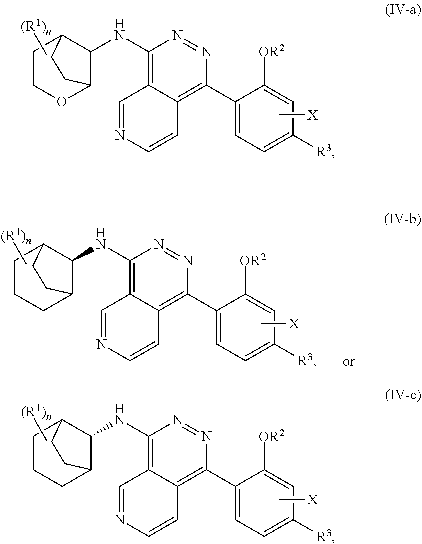

or a pharmaceutically acceptable salt or isotopically labeled derivative thereof.

Exemplary Embodiment 19A. The compound of any one of Exemplary Embodiments 1A-15A, wherein the compound is of Formula (IV-a1), (IV-b1), or (IV-c1):

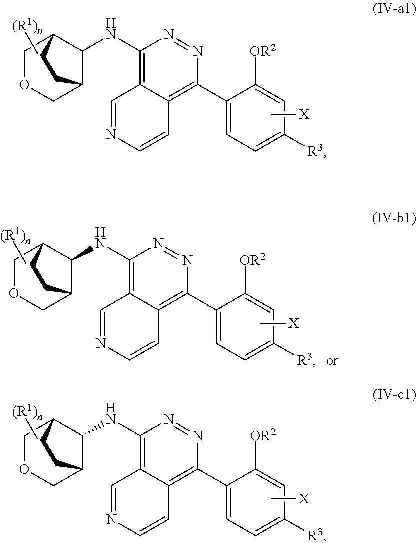

or a pharmaceutically acceptable salt or isotopically labeled derivative thereof.

Exemplary Embodiment 20A. The compound of any one of Exemplary Embodiments 1A-15A, wherein the compound is of Formula (V-a), (V-b), or (V-c):

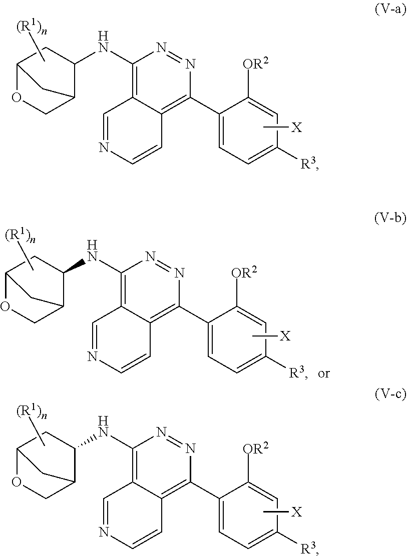

or a pharmaceutically acceptable salt or isotopically labeled derivative thereof.

Exemplary Embodiment 21A. The compound of any one of Exemplary Embodiments 1A-15A, wherein the compound is of Formula (V-a1), (V-b1), (V-c1), (V-a2), (V-b2), or (V-c2):

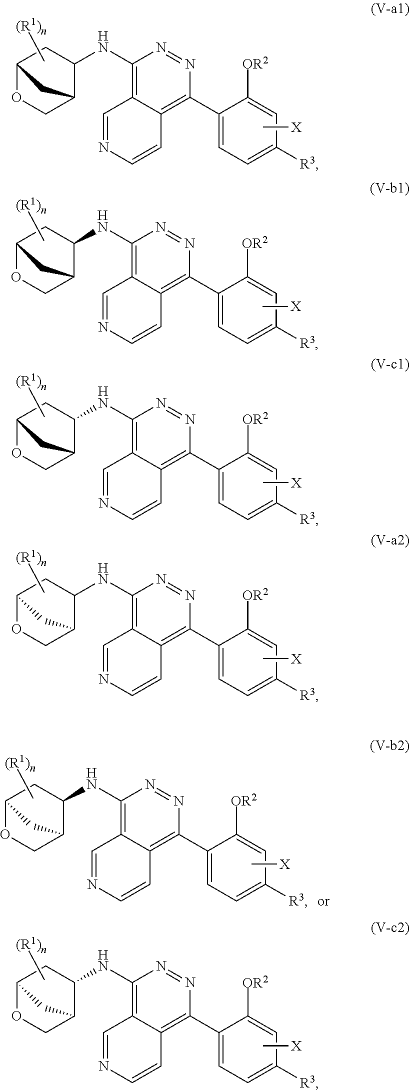

or a pharmaceutically acceptable salt or isotopically labeled derivative thereof.

Exemplary Embodiment 22A. The compound of any one of Exemplary Embodiments 1A-15A, wherein the compound is of Formula (VIf-a), (VI-b), or (VI-c):

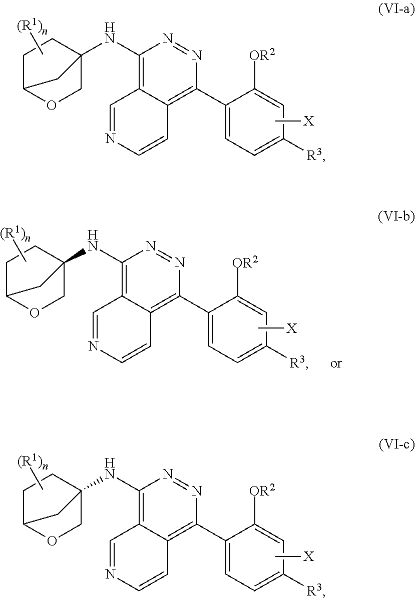

or a pharmaceutically acceptable salt or isotopically labeled derivative thereof.

Exemplary Embodiment 23A. The compound of any one of Exemplary Embodiments 1A-15A, wherein the compound is of Formula (VII-a), (VII-b), or (VII-c):

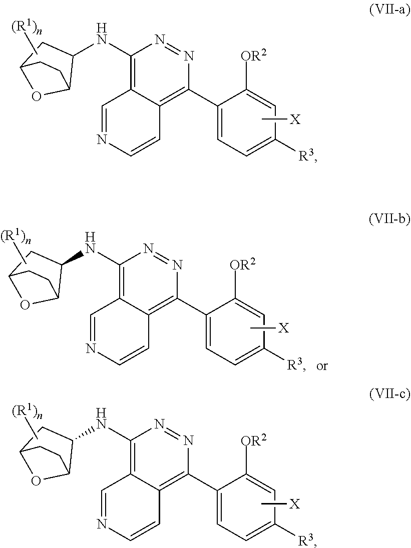

or a pharmaceutically acceptable salt or isotopically labeled derivative thereof.

Exemplary Embodiment 24A. The compound of any one of Exemplary Embodiments 1A-15A, wherein the compound is of Formula (VII-a1), (VII-b1), (VII-c1), (VII-a2), (VII-b2), or (VII-c2):

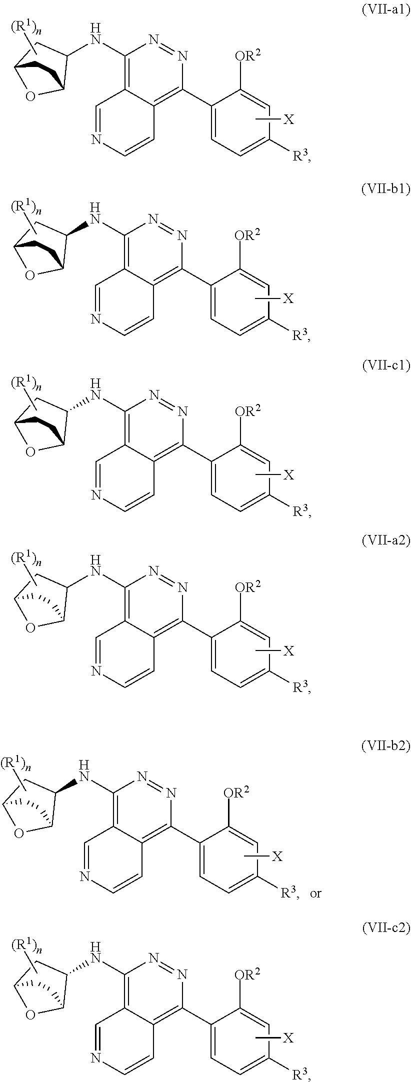

or a pharmaceutically acceptable salt or isotopically labeled derivative thereof.

Exemplary Embodiment 25A. The compound of any one of Exemplary Embodiments 1A-14A or 16A-24A, or a pharmaceutically acceptable salt or isotopically labeled derivative thereof, wherein X is halogen or C₁₋₆ alkyl independently substituted with 0, 1, 2, or 3 halogen atoms, further wherein X is located at the ortho or meta position relative to the —OR² group.

Exemplary Embodiment 26A. The compound of Exemplary Embodiment 1A selected from the group consisting of a compound of Table 1. Table 2, or Table 3, or a pharmaceutically acceptable salt thereof or isotopically labeled derivative thereof.

Exemplary Embodiment 27A. The compound of any one of the preceding Exemplary Embodiments, or a pharmaceutically acceptable salt or isotopically labeled derivative thereof, wherein the compound has a Kpu,u>0.3.

Exemplary Embodiment 28A. The compound of any one of the preceding Exemplary Embodiments, or a pharmaceutically acceptable salt or isotopically labeled derivative thereof, wherein the compound has a Kpu,u>0.3 to about 10.

Exemplary Embodiment 29A. The compound of any one of Exemplary Embodiments 1A-26A. or a pharmaceutically acceptable salt or isotopically labeled derivative thereof, wherein the compound has a Kpu,u≤0.3.

Exemplary Embodiment 30A. A pharmaceutical composition comprising the compound of any one of the preceding Exemplary Embodiments, or a pharmaceutically acceptable salt or isotopically labeled derivative thereof, and one or more pharmaceutically acceptable excipients.

Exemplary Embodiment 31A. A method of modulating NLRP3, the method comprising administering to the subject a compound of any one of Exemplary Embodiments 1A-29A, or a pharmaceutically acceptable salt or isotopically labeled derivative thereof, or a pharmaceutical composition of Exemplary Embodiment 30A.

Exemplary Embodiment 32A. A method of treating a disease or disorder, the method comprising administering to the subject a compound of any one of Exemplary Embodiments 1A-29A, or a pharmaceutically acceptable salt or isotopically labeled derivative thereof, or a pharmaceutical composition of Exemplary Embodiment 30A.

Exemplary Embodiment 33A. The compound of any one of Exemplary Embodiments 1A-29A, or a pharmaceutically acceptable salt or isotopically labeled derivative thereof, or a pharmaceutical composition of Exemplary Embodiment 30A, for use in treating a disease or disorder.

Exemplary Embodiment 34A. Use of the compound of any one of Exemplary Embodiments 1A-29A, or a pharmaceutically acceptable salt or isotopically labeled derivative thereof, or a pharmaceutical composition of Exemplary Embodiment 30A, in the manufacture of a medicament, for the treatment of a disease or disorder.

Exemplary Embodiment 35A. Use of the compound of any one of Exemplary Embodiments 1A-29A, or a pharmaceutically acceptable salt or isotopically labeled derivative thereof, or a pharmaceutical composition of Exemplary Embodiment 30A, for the treatment of a disease or disorder.

Exemplary Embodiment 36A. The method, compound, or use of any one of Exemplary Embodiments 32A-35A, wherein the disease or disorder is an NLRP3-related disease or disorder.

Exemplary Embodiment 37A. The method, compound, or use of any one of Exemplary Embodiments 32A-36A, wherein the subject is a human.

Exemplary Embodiment 38A. The method, compound, or use of any one of Exemplary Embodiments 32A-37A, wherein the disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an NLRP3-related disease.

Exemplary Embodiment 39A. The method, compound, or use of Exemplary Embodiment 38A, wherein the disease or disorder of the central nervous system is Parkinson's disease, Alzheimer's disease, traumatic brain injury, spinal cord injury, amyotrophic lateral sclerosis, or multiple sclerosis.

Exemplary Embodiment 40A. The method, compound, or use of Exemplary Embodiment 38. A, wherein the kidney disease is an acute kidney disease, a chronic kidney disease, or a rare kidney disease.

Exemplary Embodiment 41A. The method, compound, or use of Exemplary Embodiment 38A, wherein the skin disease is psoriasis, hidradenitis suppurativa (HS), or atopic dermatitis.

Exemplary Embodiment 42A. The method, compound, or use of Exemplary Embodiment 38A, wherein the rheumatic disease is dermatomyositis, Still's disease, or juvenile idiopathic arthritis.

Exemplary Embodiment 43A. The method, compound, or use of Exemplary Embodiment 38A, wherein the NLRP3-related disease is in a subject that has been determined to carry a germline or somatic non-silent mutation in NLRP3.

Exemplary Embodiment 44A. The method, compound, or use of Exemplary Embodiment 43A, wherein the NLRP3-related disease is in a subject that has been determined to carry a germline or somatic non-silent mutation in NLRP3 is cryopyrin-associated autoinflammatory syndrome.

Exemplary Embodiment 45A. The method, compound, or use of Exemplary Embodiment 44A, wherein the cryopyrin-associated autoinflammatory syndrome is familial cold autoinflammatory syndrome. Muckle-Wells syndrome, or neonatal onset multisystem inflammatory disease.

Exemplary Embodiment 46A. A method of preparing a compound of Formula (I):

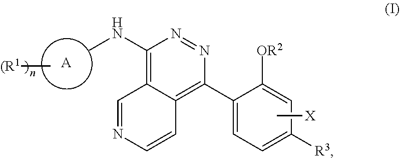

or a pharmaceutically acceptable salt or isotopically labeled derivative thereof; wherein Ring A, R¹, R², R³, X, and n are as defined in Exemplary Embodiment 1A, the method comprising reacting an amine of formula (i), or salt or isotopically labeled derivative thereof, with a compound of formula (x), or salt or isotopically labeled derivative thereof:

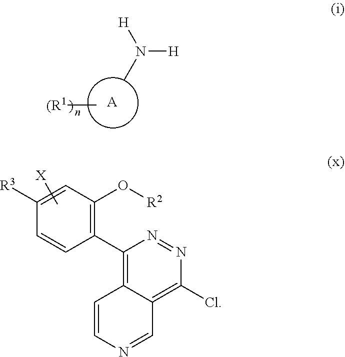

Exemplary Embodiment 47A. The method of Exemplary Embodiment 46A, further comprising treating a compound of formula (ix), or salt or isotopically labeled derivative thereof, with a chlorinating agent to provide a compound of formula (x), salt or isotopically labeled derivative thereof:

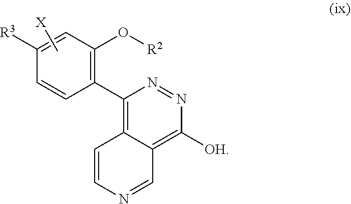

Exemplary Embodiment 48A. The method of Exemplary Embodiment 47A, further comprising treating a compound of formula (viii), or salt or isotopically labeled derivative thereof, with a chlorinating agent, followed by condensation with hydrazine, to provide a compound of formula (ix), or salt or isotopically labeled derivative thereof:

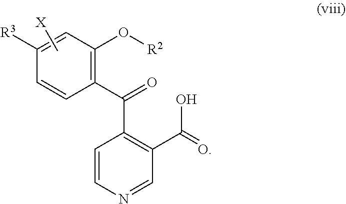

Exemplary Embodiment 49A. The method of Exemplary Embodiment 48A, further comprising treating a 3,4-pyridinedicarboxylic acid anhydride of formula (vii), or salt or isotopically labeled derivative thereof, with a Grignard reagent of formula (xx), or salt or isotopically labeled derivative thereof, to provide a compound of formula (viii), or salt or isotopically labeled derivative thereof:

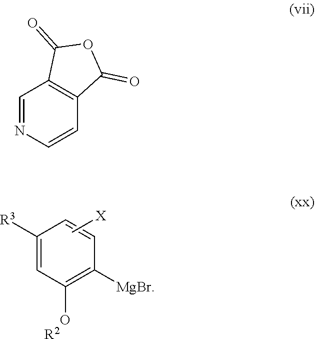

Exemplary Embodiment 50A. A method of preparing a compound of Formula (I):

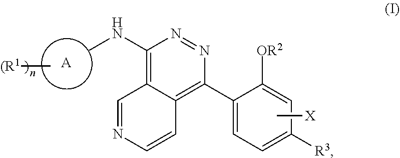

or a pharmaceutically acceptable salt or isotopically labeled derivative thereof, wherein Ring A, R¹. R², R³, X, and n are as defined in Exemplary Embodiment 1A, the method comprising reacting a boronic acid or boronate of formula (iv), or salt or isotopically labeled derivative thereof, with a compound of formula (iii), or salt or isotopically labeled derivative thereof:

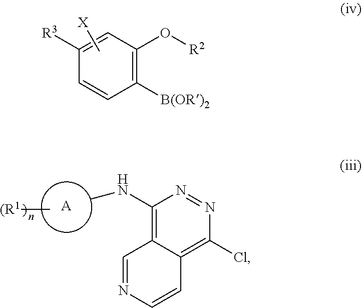

wherein R′ is H or C₁₋₆ alkyl, or two R′ groups are joined via a C₂-C₃ alkylene linker optionally substituted with one or more C₁₋₃ alkyl or C₁₋₃ haloalkyl.

Exemplary Embodiment 51A. The method of Exemplary Embodiment 50A, further comprising treating a heteroaryl dichloride of formula (ii), or salt or isotopically labeled derivative thereof, with an amine of formula (i), or salt or isotopically labeled derivative thereof, to provide a compound of Formula (iii), or salt or isotopically labeled derivative thereof:

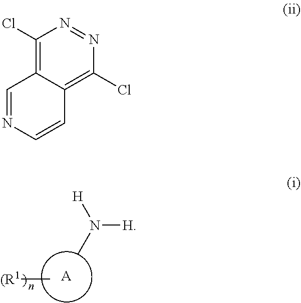

Exemplary Embodiment 52A. The method of Exemplary Embodiment 46A or 50A, wherein R² is C₁-C₆ alkyl or —C(O)(C₁-C₆ alkyl), and wherein alkyl is optionally substituted with one or more halogen atoms, the method further comprising deprotecting the compound of Formula (I), or a pharmaceutically acceptable salt or isotopically labeled derivative thereof, to provide a compound of Formula (I), or a pharmaceutically acceptable salt or isotopically labeled derivative thereof, wherein R² is H.

### Examples

In order that this disclosure may be more fully understood, the following Examples are set forth. It should be understood that these examples are for illustrative purposes only and are not to be construed as limiting this disclosure in any manner.

For exemplary purpose, neutral (free base) compounds described herein are synthesized and tested in the examples. It is understood that the neutral compounds disclosed herein may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

### Synthetic Methods

Nuclear magnetic resonance (NMR) spectra were recorded at 400 MHz as stated and at 300.3 K unless otherwise stated: the chemical shifts (8) are reported in parts per million (ppm). Spectra were recorded using a Bruker Avance 400 instrument with 8, 16 or 32 scans.

Liquid Chromatography-Mass Spectrometry (LCMS) chromatograms and spectra were recorded using a Shimadzu LCMS-2020. Injection volumes were 0.7-8.0 μl and the flow rates were typically 0.8 or 1.2 mL/min. Detection methods were diode array (DAD) or evaporative light scattering (ELSD) as well as positive ion electrospray ionization. MS range was 100-1000 Da. Solvents were gradients of water and acetonitrile (MeCN) both containing a modifier (typically 0.01-0.04%) such as trifluoroacetic acid (TFA), formic acid (FA) or ammonium carbonate. ESI=electrospray ionization; m/z=mass/charge; RT=retention time (minutes).

Gas Chromatography-Mass Spectrometry (GCMS) chromatograms and spectra were recorded using Agilent GCMS 8890-5977 and Detector Channel FID. GC Parameters: DB-5 MS, 12m×0.20 mm×0.33 um; Column Oven Temp: 50.0; Injection volume: 0.5 μL: Column Flow: 1.2 ml/min; Injection temperature: 300° C.; Injection Mode: Split; Split Ratio: 20:1; Detector temperature: 300° C.; Initial temperature: 50° C., for 1 min then 40° C./min to 300° C., for 1.75 min. Makeup Gas: He; Makeup Flow: 25.0 mL/min; H2; Flow: 30.0 mL/min: Air Flow: 400.0 mL/min; Final temperature: 300° C. The MS detector of acquisition mode: Start Time: 2.00 min; End Time: 9.00 min; Acquisition Mode: Sean; Interface Type: EI Threshold: 150; Scan Speed: 1562; Start m/z: 50.00; End m/z: 550.00; MS Source: 230.00° C.; MS Quad: 150.00° C.; Solvent Cut Time: 2.00 min.

Purification/Separation Methods. Provided in the below Table A are purification and/or separation methods employed in the synthesis and isolation of the compounds of the present disclosure. Rf=retention factor; RT=retention time (minutes).

If a stereochemical position is arbitrarily assigned, an Asterix (*) is included as part of the compound number. If a stereochemical position is rationally assigned, an Asterix and dashed “Y” (*-r) is included as part of the compound number. If the absolute stereochemistry has been determined or is retroactively assigned based on that known stereochemistry, no Asterix or dashed “r” (*-r) is included. Rational assignment signifies there is a correlation between the designated assignment and a known absolute assignment.

TABLE A
Purification/Separation Methods

| Method | Conditions |
|---|---|
| A | XBridge Shield RP18 OBD Column, 19*250 mm, 10 μm; Mobile phase: water (10 |
| | mmol/L NH₄HCO₃) and acetonitrile (MeCN) (hold 39% MeCN in 17 min); Wave |
| | Length: UV 254/220 nm |
| B | XBridge Prep OBD C18 Column, 30*150 mm, 5 μm; Mobile Phase A: water (10 mmol/L |
| | NH₄HCO₃), Mobile Phase B: acetonitrile; Flow rate: 60 mL/min; Gradient: 29% B to |
| | 34% B in δ min, 34% B; Wave Length: 254 nm |
| C | Reverse phase flash column: C18 silica gel; Mobile phase: acetonitrile in water |
| | (10 mmol/L NH₄HCO₃), 0% to 100% gradient in 30 min; Wave Length: 254 nm |
| D | Reverse phase flash column: C18 silica gel; mobile phase, MeCN in Water (0.1% |
| | trifluoroacetic acid (TFA)), 0% to 100% gradient in 30 min; Wave Length: 254 nm |
| E | CH1RAL ART Amylose-SA, 2#25 cm, 5 μm; Mobile phase: Hexanes (0.1% TFA) and |
| | methanol:dichloromethane (MeOH:DCM) = 1:1 (hold 25% MeOH:DCM = 1:1 in 14.5 |
| | min); Wave Length: 254 nm |
| F | CH1RALPAK IG, 2*25 cm, 5 um; mobile phase, hexanes (Hex) (0.1% trifluoroacetic |
| | acid (TFA)) and isopropyl alcohol (IPA) (hold 40% IPA- in 16 min); Wave Length: 254 |
| | nm |
| G | CH1RALPAK ID, 2*25 cm, 5 um; mobile phase, Hexanes (0.2% formic acid (FA)) and |
| | IPA:DCM = 1:1- (hold 60% IPA:DCM = 1:1 in 23 min); Detector, UV 254 nm |
| H | CH1RAL ART Cellulose-SC, 2*25 cm, 5 um; mobile phase, Hexanes (0.5% 2M NH₃ in |
| | MeOH) and MeOH:DCM = 1:1- (hold 60% MeOH:DCM = 1:1- in 10 min); Wave Length: |
| | 254 nm |
| I | Reverse phase flash column: C18 silica gel; mobile phase, MeCN in Water (0.1% |
| | NH₃•H₂O), 0% to 100% gradient in 30 min; Wave Length: 254 nm |
| J | YMC-Actus Triart C18 ExRS, 30*150 mm, 5 μm; Mobile Phase A: Water (10 mmol/L |
| | NHAHCO₃), Mobile Phase B: acetonitrile; Flow rate: 60 mL/min; Gradient: 23% B to |
| | 33% B in 10 min, 33% B: Wave Length: 254 nm |
| K | XSelect CSH Prep C18 OBD Column, 19*250 mm, 5 μm; mobile phase, Water (10 |
| | mmol/L NH₄HCO₃) and acetonitrile (37% MeCN up to 42% in 10 min); Wave Length: |
| | 254 nm |
| L | XBridge Prep Phenyl OBD Column, 19*250 mm, 5 μm; mobile phase, Water (10 mmol/L |
| | NH₄HCO₃) and MeOH (40% MeOH up to 60% in 10 min); Wave Length: 254 nm |
| M | Reverse phase flash column: C18 silica gel; mobile phase: MeCN in water, 10% to 50% |
| | gradient in 30 min; Wave Length: 254 nm |
| N | Reverse phase flash column: C18 silica gel; mobile phase: MeCN in Water, 10% to 50% |
| | gradient in 20 min; Wave Length: 254 nm |
| O | XBridge Prep Phenyl OBD Column, 19*250 mm, 5 μm; mobile phase, Water (10 mmol/L |
| | NH₄HCO₃) and MeCN (35% MeCN up to 45% in 10 min); Wave Length: 254 nm |
| P | XBridge Shield RP18 OBD Column, 30*150 mm, 5 μm; Mobile Phase A: Water (10 |
| | mmol/L NH₄HCO₃), Mobile Phase B: MeCN; Flow rate: 60 mL/min; Gradient: 35% B to |
| | 45% B in δ min, 45% B; Wave Length: 254 nm |
| Q | XBridge Prep Phenyl OBD Column, 19*150 mm, 5 μm; Mobile Phase A: Water (10 |
| | mmol/L NH₄HCO₃), Mobile Phase B: MeCN; Flow rate: 60 mL/min; Gradient: 27% B to |
| | 32% B in δ min, 32% B; Wave Length: 254 nm |
| R | CH1RAL ART Amylose-SA, 2*25 cm, 5 um; mobile phase, Hexanes (0.2% FA) and |
| | MeOH:DCM = 1:1- (hold 15% MeOH:DCM = 1:1- in 31 min); Wave Length: 254 nm |
| S | CH1RALPAK 1H, 2*25 cm, 5 um; mobile phase, Hexanes (0.2% FA) and IPA:DCM = 1:1- |
| | (hold 25% IPA:DCM = 1:1- in 13 min); Wave Length: 254 nm |
| T | CH1RALPAK IF, 2*25 cm, 5 um; mobile phase, Hexanes (0.5% 2M NH₃-MeOH) and |
| | MeOH:DCM = 1:1- (hold 40% MeOH:DCM = 1:1 in 15 min); Wave Length: 254 nm |
| U | CH1RAL ART Cellulose-SC, 2*25 cm, 5 um; mobile phase, Hexanes (0.5% 2M NH₃- |
| | MeOH)- and EtOH:DCM = 1:1 (hold 30% EtOH:DCM = 1:1- in 15 min); Wave Length: 254 |
| | nm |
| V | Lux 5 um Cellulose-4 column, 2.12*25 cm, 5 μm; Mobile Phase A: Hexanes (0.1% FA); |
| | Mobile Phase B: EtOH; Flow rate: 20 mL/min; Gradient: 15% B to 15% B in 12 min; |
| | Wave Length: 220/254 nm |
| W | CH1RALPAK IG, 2*25 cm, 5 μm; Mobile Phase A: Hexanes (0.5% 2M NH₃ in MeOH); |
| | Mobile Phase B: IPA:DCM = 1:1 -- HPLC; Flow rate: 20 mL/min; Gradient: 25% B to 25% |
| | B in 19 min; Wave Length: 220/254 nm |
| X | XBridge Prep OBD C18 Column, 30*150 mm, 5 μm; mobile phase, Water (10 mmol/L |
| | NH₄HCO₃) and MeCN (27% up to 37% in 10 min); Wave Length: 254 nm |
| Y | XBridge Prep Phenyl OBD Column, 19*250 mm, 5 μm; Mobile Phase A: Water (10 |
| | mmol/L NH₄HCO₃), Mobile Phase B: MeCN; Flow rate: 60 mL/min; Gradient: 20% B to |
| | 30% B in 10 min, 30% B; Wave Length: 254 nm |
| Z | Reverse phase flash column: C18 silica gel; mobile phase, acetonitrile in water, 90% to |
| | 100% gradient in 15 min; detector, UV 220 nm |
| AA | Reverse phase flash column: C18 silica gel; mobile phase, acetonitrile in water, 50% to |
| | 60% gradient in 10 min; detector, UV 220 nm |
| BB | Reverse phase flash column: C18 silica gel; mobile phase, acetonitrile in water, 20% to |
| | 40% gradient in 15 min; detector, UV 254 nm |
| CC | Reverse phase flash column: C18 silica gel; mobile phase, acetonitrile in water, 30% to |
| | 50% gradient in 10 min; detector, UV 254/220 nm |
| DD | XBridge Prep Phenyl OBD Column 19*250 mm 5 μm; Mobile Phase A: Water (50 mmol/L |
| | NH₄HCO₃), Mobile Phase B: MeOH; Flow rate: 25 mL/min; Gradient: 50% B to 62% B in |
| | 10 min; Wave Length: 254/220 nm |
| EE | XBridge Prep Phenyl OBD Column 19*250 mm, 5 μm; Mobile Phase A: Water (50 mmol/L |
| | NH₄HCO₃), Mobile Phase B: acetonitrile; Flow rate: 25 mL/min; Gradient: 25% B to 35% |
| | B in 15 min; Wave Length: 254/233 nm |
| FF | Kinetex EVO C18, 21.2*250 mm, 5 μm; Mobile Phase A: Water (10 mmol/L NH₄HCO₃), |
| | Mobile Phase B: acetonitrile; Flow rate: 20 mL/min; Gradient: 30% B to 40% B in 15 min; |
| | Wave Length: 254/220 nm. |
| GG | Reverse phase flash column: C18 silica gel; mobile phase, acetonitrile in water, 10% to |
| | 30% gradient in 15 min; detector, UV 254/220 nm |
| HH | XBridge Prep Phenyl OBD Column 19*250 mm, 5 μm; Mobile Phase A: Water (10 mmol/L |
| | NH₄HCO₃), Mobile Phase B: acetonitrile; Flow rate: 20 mL/min; Gradient: 20% B to 30% |
| | B in 10 min; Wave Length: 254/220 nm |
| II | XBridge Prep Phenyl OBD Column 19*250 mm, 5 μm; Mobile Phase A: Water(10 mmol/L |
| | NH₄HCO₃), Mobile Phase B: acetonitrile; Flow rate: 20 mL/min; Gradient: 15% B to 25% |
| | B in 10 min; Wave Length: 254/220 nm |
| JJ | Reverse phase flash column: C18 silica gel; mobile phase, acetonitrile in water (1:3); |
| | detector, UV 254 nm |
| KK | XSelect CSH Prep C18 OBD Column, 19*250 mm, 5 μm; Mobile Phase A: Water(10 |
| | mmol/L NH₄HCO₃), Mobile Phase B: ACN; Flow rate: 20 mL/min; Gradient: 16% B to |
| | 21% B in 10 min, 21% B; Wave Length: 254 nm |
| LL | C18 silica gel; mobile phase, acetonitrile in water, 50% to 70% gradient in 10 min; |
| | detector, UV 254 nm. |
| MM | XSelect CSH Prep C18 OBD Column, 19*250 mm, 5 μm; Mobile Phase A: Water (0.05% |
| | TFA), Mobile Phase B: MeOH; Flow rate: 60 mL/min; Gradient: 45% B to 50% B in 11 |
| | min, 50% B; Wave Length: 254 nm |
| NN | Lux 5 um Cellulose-4, 2.12*25 cm, 5 μm; Mobile Phase A: Hexanes (0.1% formic acid), |
| | Mobile Phase B: EtOH; Flow rate: 20 mL/min; Gradient: 15% B to 15% B in 12 min; |
| | Wave Length: 220/254 nm |
| OO | CH1RALPAK IG, 2*25 cm, 5 μm; Mobile Phase A: Hex(0.5% 2M NH3-MeOH), Mobile |
| | Phase B: IPA: DCM = 1:1; Flow rate: 20 mL/min; Gradient: 25% B to 25% B in 19 min; |
| | Wave Length: 220/254 nm |

### Protocol A Examples

### Example 1. 2-(4-(((1R,3r,5S)-8-oxabicyclo[3.2.1]octan-3-yl)amino)pyrido[3,4-d]pyridazin-1-yl)-5-chlorophenol (Compound 1A*-r)

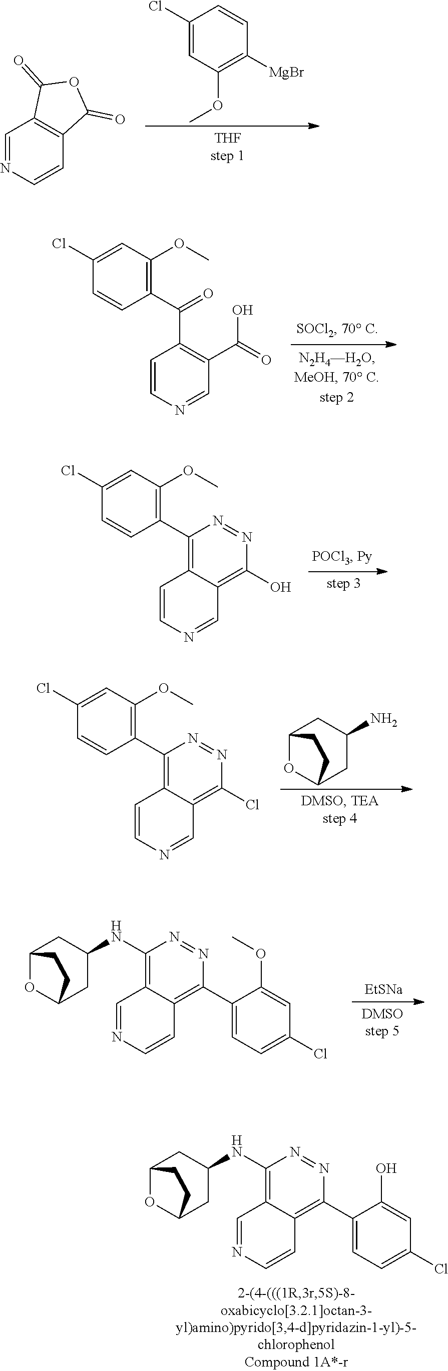

Step 1. To a stirred solution of furo[3,4-c]pyridine-1,3-dione (30.0 g, 201 mmol, 1 equiv) and tetrahydrofuran (THF) (300 mL) was added bromo (4-chloro-2-methoxyphenyl) magnesium (0.5 M in THF) (241 mL, 120 mmol, 0.6 equiv) dropwise at −78° C. under nitrogen atmosphere. The resulting mixture was stirred for 2 h at 25° C. under nitrogen atmosphere. The reaction was quenched by the addition into an aqueous solution of NH₄Cl (500 mL) and ethyl acetate (EtOAc) (500 mL), and the reaction mixture was extracted with EtOAc (3×500 mL). The combined organic layers were washed with H₂O (1×500 mL), dried over anhydrous Na₂SO₄, filtered, and the filtrate was concentrated under reduced pressure to provide crude 4-(4-chloro-2-methoxybenzoyl)pyridine-3-carboxylic acid (20 g, 34% yield) which was taken on to the next step as a crude isolate. LCMS (ES, m/z): RT=0.662 min, m/z=292.0 [M+1]⁺.

Step 2. Into a 250 mL round-bottom flask was added 4-(4-chloro-2-methoxybenzoyl)pyridine-3-carboxylic acid (5 g, 17.1 mmol, 1 equiv) and SOCH (50 mL). The resulting mixture was stirred for 2 h at 70° C. The reaction was monitored by thin layer chromatography (TLC). After the reaction was completed, the resulting mixture was concentrated under vacuum. The residue was dissolved in dichloromethane (DCM) (50 mL) and added into the solution of NH₂NH₂·H₂O (3.43 g, 68.6 mmol, 4 equiv), methanol (MeOH) (50 mL) at 0° C. The resulting mixture was stirred for 3 h at 70° C., in an oil bath. The reaction progress was monitored by LCMS. The precipitated solids were collected by filtration. The crude product (4 g. 90% purity) was purified by preparative high performance liquid chromatography (prep-HPLC) Method A to provide 1-(4-chloro-2-methoxyphenyl)pyrido[3,4-d]pyridazin-4-ol (2 g, 41% yield). LCMS: (ES, m/z): RT=0.723 min, m/z=288.0 [M+H]⁺. ¹H NMR (400 MHz, dimethylsulfoxide-d6 (DMSO-d₆)) δ 12.90 (s, 1H), 9.50 (s, 1H), 8.94 (d, J=5.5 Hz, 1H), 7.41 (d, J=8.0 Hz, 1H), 7.33 (d, J=1.9 Hz, 1H), 7.23-7.15 (m, 2H), 3.75 (s, 3H).

Step 3. Into a 250 mL round-bottom flask was added 1-(4-chloro-2-methoxyphenyl)pyrido[3,4-d]pyridazin-4-ol (2.50 g. 8.69 mmol, 1 equiv), POCl₃ (40 mL), and pyridine (Py) (4 mL). The resulting mixture was stirred for 3 h at 100° C. The reaction progress was monitored by LCMS. The reaction was quenched with 500 ml of sodium bicarbonate (aq.) and 500 mL of ethyl acetate (EtOAc) at 0° C. The resulting mixture was extracted with EtOAc (3×500 mL). The combined organic layers were dried over anhydrous Na₂SO₄, filtered and the filtrate was concentrated under reduced pressure to provide 4-chloro-1-(4-chloro-2-methoxyphenyl)pyrido[3,4-d]pyridazine (1.5 g, 57% yield). LCMS (ES, m/z): RT=0.845 min, m/z=306.0 [M+1]⁺. ¹H NMR (400 MHZ, DMSO-d₆) δ 9.84-9.68 (m, 1H), 9.12 (d, J=5.7 Hz, 1H), 7.60-7.56 (m, 1H), 7.51 (d, J=8.1 Hz, 1H), 7.41 (d, J=1.9 Hz, 1H), 7.32-7.25 (m, 1H), 3.74 (s, 3H).

Step 4. Into a 8 mL vial was added 4-chloro-1-(4-chloro-2-methoxyphenyl)pyrido[3,4-d]pyridazine (50.0 mg. 0.16 mmol, 1 equiv), commercially available (1R,3r,5S)-8-oxabicyclo[3.2.1]octan-3-amine (24.9 mg, 0.190 mmol, 1.20 equiv), triethylamine (TEA) (49.6 mg. 0.480 mmol, 3 equiv) and dimethylsulfoxide (DMSO) (1 mL), and the reaction mixture was stirred for 4 h at 80° C., which was monitored by LCMS. The reaction was added water (10 mL) and ethyl acetate (EtOAc) (10 mL), and the reaction mixture was extracted with EtOAc (3×10 mL). The combined organic layers were washed with H₂O (1×20 mL), dried over anhydrous Na₂SO₄, filtered, and the filtrate was concentrated under reduced pressure to provide crude N-(8-oxabicyclo[3.2.1]octan-3-yl)-1-(4-chloro-2-methoxyphenyl)pyrido[3,4-d]pyridazin-4-amine which was taken on to the next step as a crude isolate. LCMS (ES, m/z): RT=0.68 min, m/z=397 [M+H]+.

Step 5. Into a 8 mL vial was added 1-(4-chloro-2-methoxyphenyl)-N-{8-oxabicyclo[3.2.1]octan-3-yl}pyrido[3,4-d]pyridazin-4-amine (100 mg, 0.25 mmol, 1 equiv), (ethylsulfanyl) sodium (493 mg, 5.00 mmol, 20 equiv) and dimethylsulfoxide (DMSO) (3 mL). The resulting mixture was stirred for 1 h at 120° C., and the reaction was monitored by LCMS. The reaction was quenched with addition of water (10 mL) and ethyl acetate (EtOAc) (10 mL), and the reaction mixture was extracted with EtOAc (3×10 mL). The combined organic layers were washed with H₂O (1×20 mL), dried over anhydrous Na₂SO₄, filtered, and the filtrate was concentrated under reduced pressure. The residue was purified by reverse flash chromatography Method M to provide the crude product, which was further purified by Prep-HPLC Method B to provide 2-(4-(((1R,3r,5S)-8-oxabicyclo[3.2.1]octan-3-yl)amino)pyrido[3,4-d]pyridazin-1-yl)-5-chlorophenol (Compound 1A*-r) (35.5 mg, 37% yield). LCMS (ES, m/z): RT=0.85 min, m/z=383 [M+H]+, ¹H NMR (400 MHZ, DMSO-d₆) δ 10.24 (s, 1H), 9.71 (s, 1H), 8.87 (d, J=5.7 Hz, 1H), 7.47 (d, J=3.4 Hz, 1H), 7.37-7.31 (m, 2H), 7.04 (d, J=6.8 Hz, 2H), 4.41-4.31 (m, 3H), 2.28-2.11 (m, 4H), 2.04 (d, J=14.4 Hz, 2H), 1.88 (m, J=6.9, 4.7, 3.1 Hz, 2H).

Stereochemistry of Compound 1A*-r was rationally assigned based on use of chiral starting material in step 4 of this example.

### Example 2. 2-(4-(((1R,3r,5S)-8-oxabicyclo[3.2.1]octan-3-yl)amino)pyrido[3,4-d]pyridazin-1-yl)-5-methylphenol (Compound 2A*-r)

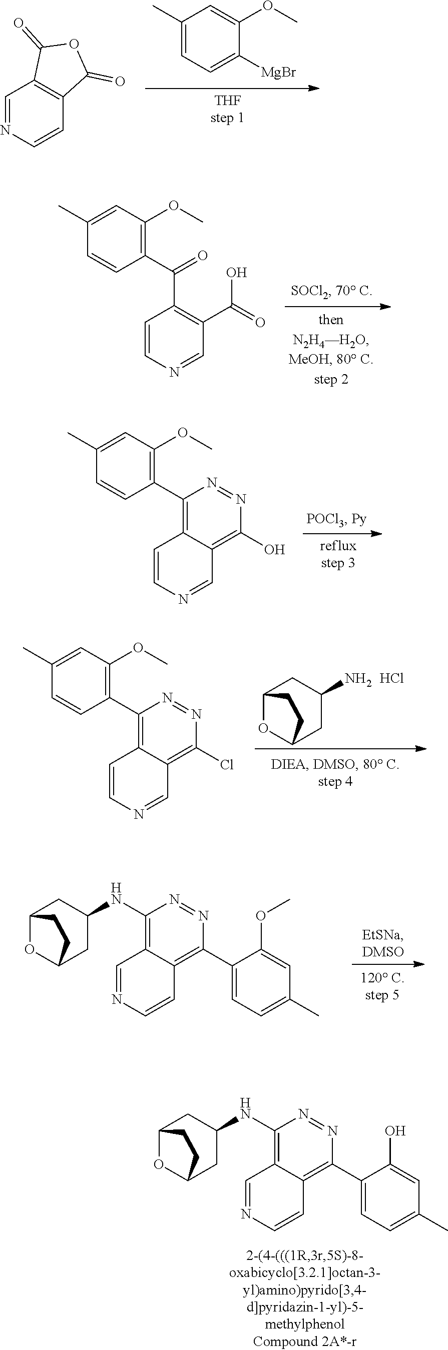

Step 1. Into a 250 mL 3-necked round-bottom flask was added furo[3,4-c]pyridine-1,3-dione (10 g. 67.06 mmol, 1 equiv) and tetrahydrofuran (THF) (100 mL), and bromo (2-methoxy-4-methylphenyl) magnesium (9 g. 40.2 mmol. 0.6 equiv) was added by dropwise at −78° C. The resulting mixture was stirred for 1 h at room temperature under nitrogen atmosphere, and the reaction progress was monitored by LCMS. The reaction was quenched by the addition of ice/water (50 mL) at room temperature, extracted with ethyl acetate (EtOAc) (3×50 mL), dried over anhydrous Na₂SO₄, filtered, and the filtrate was concentrated under reduced pressure to provide 4-(2-methoxy-4-methylbenzoyl)pyridine-3-carboxylic acid (6 g, 33% yield). LCMS: (ES, m/z): RT=0.570 min, m/z=272 [M+J]⁺.

Step 2. Into a 250 mL round-bottom flask was added 4-(2-methoxy-4-methylbenzoyl)pyridine-3-carboxylic acid (2 g, 7.37 mmol, 1 equiv) and SOCl₂ (20 mL), and the resulting mixture was stirred for 2 h at 70° C., which was monitored by TLC. After the reaction was completed, the resulting mixture was concentrated under vacuum, the residue was dissolved in dichloromethane (DCM) (50 mL) and added into a solution of hydrazine hydrate (1.7 g. 34.0 mmol. 4.61 equiv) and ethanol (EtOH) (10 mL) at 0° C., and the resulting mixture was then stirred for 3 h at 70° C., which was monitored by LCMS. After completion of the reaction, the precipitated solids were collected by filtration to provide the crude title compound (2.0 g, 80% purity) which was purified by Prep-HPLC Method H to provide 1-(2-methoxy-4-methylphenyl)pyrido[3,4-d]pyridazin-4-ol (1.30 g, 66.0% yield). LCMS: (ES, m/z): RT=0.880 min, m/z=268 [M+1]+. ¹H NMR (400 MHZ, DMSO-d₆) δ 12.75 (s, 1H), 9.49 (d, J=0.9 Hz, 1H), 8.93 (d, J=5.5 Hz, 1H), 7.25 (d, J=7.6 Hz, 1H), 7.17 (d, J=5.5, 0.9 Hz, 1H), 7.06 (s, 1H), 6.98-6.91 (m, 1H), 3.70 (s. 3H), 2.43 (s, 3H).

Step 3. Into a 250 mL round-bottom flask was added 1-(2-methoxy-4-methylphenyl)pyrido[3,4-d]pyridazin-4-ol (800 mg. 2.99 mmol, 1 equiv), POCl₃ (10 mL), and pyridine (1 mL) at room temperature. The resulting mixture was stirred for 2 h at 110° C. under nitrogen atmosphere, and the reaction progress was monitored by LCMS. The reaction was quenched by the addition into aqueous of NaHCO₃ (500 mL) and ethyl acetate (EtOAc) (500 mL) at 0° C., and the reaction mixture was extracted with EtOAc (3×500 mL). The combined organic layers were washed with H₂O (1×500 mL), dried over anhydrous Na₂SO₄, filtered, and the filtrate was concentrated under reduced pressure to provide 4-chloro-1-(2-methoxy-4-methylphenyl)pyrido[3,4-d]pyridazine (300 mg. 35.1% yield). LCMS: (ES, m/z): RT=0.837 min, m/z=286 [M+1]+. 1H NMR (300 MHz, DMSO-d₆) δ 9.74 (d, J=1.0 Hz. 1H), 9.10 (d, J=5.7 Hz, 1H), 7.59-7.46 (m, 1H), 7.35 (d, J=7.6 Hz, 1H), 7.14 (d, J=1.4 Hz, 1H), 7.08-6.95 (m, 1H), 3.69 (s, 3H), 2.47 (s, 3H).

Step 4. Into a 8 mL vial was added 4-chloro-1-(2-methoxy-4-methylphenyl)pyrido[3,4-d]pyridazine (100 mg. 0.35 mmol, 1 equiv), commercially available (1R,3r,5S)-8-oxabicyclo[3.2.1]octan-3-amine hydrochloride (90 mg, 0.55 mmol, 1.57 equiv), diisopropylethylamine (DIEA) (361.87 mg, 2.80 mmol, 8 equiv) and dimethylsulfoxide (DMSO) (1.2 mL) at room temperature. The resulting mixture was stirred for 16 h at 80° C. The reaction progress was monitored by LCMS. The residue was purified by reverse flash chromatography Method I to provide N-((1R,3r,5S)-8-oxabicyclo[3.2.1]octan-3-yl)-1-(2-methoxy-4-methylphenyl)pyrido[3,4-d]pyridazin-4-amine (60 mg. 45% yield). LCMS (ES, m/z): RT=0.628 min, m/z=377 [M+1]⁺. Stereochemistry was rationally assigned based on use of chiral starting material.

Step 5. Into a 8 mL vial was added N-((1R,3r,5S)-8-oxabicyclo[3.2.1]octan-3-yl)-1-(2-methoxy-4-methylphenyl)pyrido[3,4-d]pyridazin-4-amine (50 mg, 0.13 mmol, 1 equiv), (ethylsulfanyl) sodium (223.42 mg, 2.66 mmol, 20 equiv) and dimethylsulfoxide (DMSO) (1.2 mL) at room temperature. The resulting mixture was stirred for 4 h at 120° C. The reaction progress was monitored by LCMS. The residue was purified by reverse flash chromatography Method I to provide the crude product (30 mg. 85 purity), which was further purified by Prep-HPLC Method J (RT (min): 9.2) to provide 2-(4-(((1R,3r,5S)-8-oxabicyclo[3.2.1]octan-3-yl)amino)pyrido[3,4-d]pyridazin-1-yl)-5-methylphenol (Compound 2A*-r) (12.7 mg, 26.3% yield). LCMS (ES, m/z): RT=0.533 min, m/z=363 [M+1]+. ¹H NMR (400 MHZ, DMSO-d₆) δ 9.70 (d, J=1.0 Hz, 1H), 9.67-9.62 (m, 1H), 8.86-8.80 (m, 1H), 7.40 (d, J=3.3 Hz, 1H), 7.34-7.26 (m, 1H), 7.21 (d, J=7.6 Hz, 1H), 6.85-6.77 (m, 2H), 4.36 (d, J=7.7 Hz. 3H), 2.34 (s, 3H), 2.24 (d, J=7.3 Hz, 2H), 2.16-2.06 (m, 2H), 2.04 (d, J=14.4 Hz, 2H), 1.87-1.76 (m, 2H).

Stereochemistry of Compound 2A*-r was rationally assigned based on use of chiral starting material in step 4 of this example, and rational stereochemical assignment of Compound 2B* in Example 3.

### Example 3. 2-(4-(((1R,3s,5S)-8-oxabicyclo[3.2.1]octan-3-yl)amino)pyrido[3,4-d]pyridazin-1-yl)-5-methylphenol (Compound 2B*-r)

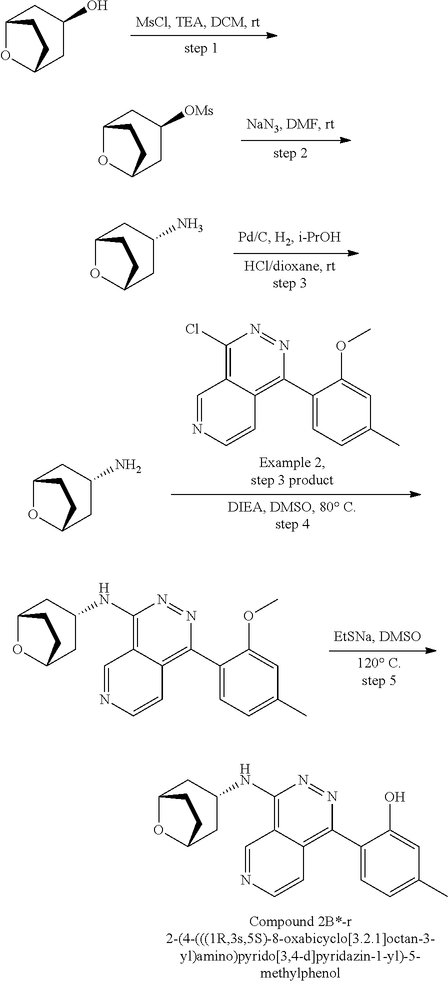

Step 1. Into a 20 mL vial was added commercially available (1R,3r,5S)-8-oxabicyclo[3.2.1]octan-3-ol (300 mg, 2.34 mmol, 1 equiv), methanesulfonyl chloride (MsCl) (322 mg. 2.81 mmol, 1.2 equiv), triethylamine (TEA) (474 mg, 4.68 mmol, 2 equiv), and dichloromethane (DCM) (4 mL) at room temperature. The resulting mixture was stirred for 1 h at room temperature under nitrogen atmosphere. The reaction progress was monitored by TLC. The resulting mixture was extracted with ethyl acetate (EtOAc) (3×100 mL). The combined organic layers were washed with water (3×100 mL.), dried over anhydrous Na₂SO₄. After filtration, the filtrate was concentrated under reduced pressure to provide (1R,3S,5S)-8-oxabicyclo[3.2.1]octan-3-yl methanesulfonate (240 mg, 34.80% yield). The crude product was used for next step without further purification. TLC: petroleum ether:ethyl acetate (PE:EtOAc)=1:1, Rf=0.7.

Step 2. Into a 20 mL vial was added (1R,3S,5S)-8-oxabicyclo[3.2.1]octan-3-yl methanesulfonate (240 mg, 1.16 mmol, 1 equiv), NaN₃ (227 mg, 3.49 mmol, 3 equiv), and N,N-dimethylformamide (DMF) (4 mL) at room temperature. The resulting mixture was stirred for 1 h at 80° C. under nitrogen atmosphere. The reaction progress was monitored by TLC. The resulting mixture was extracted with ethyl acetate (EtOAc) (3×100 mL), and the combined organic lavers were washed with water (3×50 mL) and dried over anhydrous Na₂SO₄. After filtration, the filtrate was concentrated under reduced pressure to provide (1R,3R,5S)-3-azido-8-oxabicyclo[3.2.1]octane (220 mg, 88.7% yield), which was used in next step without further purification. TLC: petroleum ether:ethyl acetate (PE:EtOAc)=1:1, Rf=0.6. Stereochemistry rationally assigned based on chiral starting material and assumed stereochemical inversion in this step.

Step 3. Into a 50 mL round-bottom flask was added (1R,3R,5S)-3-azido-8-oxabicyclo[3.2.1]octane (220 mg, 1.44 mmol, 1 equiv), Pd/C (73.36 mg. 0.70 mmol, 0.48 equiv), and isopropanol (i-PrOH) (2 mL) at room temperature. The resulting mixture was stirred for 1 h at room temperature under hydrogen atmosphere. The reaction progress was monitored by LCMS. To the above mixture was added HCl (gas) in 1,4-dioxane (3 mL) dropwise over Imin at room temperature. The resulting mixture was stirred for additional 0.5 h at room temperature. The resulting mixture was concentrated under reduced pressure to provide (1R,3s,5S)-8-oxabicyclo[3.2.1]octan-3-amine (180 mg, 69.0% yield). LCMS: (ES, m/z): RT=1.233 min, m/z=128 [M+1]+. 1H NMR (400 MHZ, DMSO-d₆) δ 8.03 (s, 2H), 4.40-4.31 (m, 1H), 4.20-4.01 (m. 1H), 3.49-3.31 (m, 2H), 1.85-1.68 (m, 2H), 1.90-1.72 (m. 2H). 1.75-1.68 (m, 1H), 1.71-1.50 (m, 2H). Stereochemistry rationally assigned based on chiral starting material and assumed stereochemical inversion in step 2.

Step 4. Into a 20 mL vial was added 4-chloro-1-(2-methoxy-4-methylphenyl)pyrido[3,4-d]pyridazine (120 mg. 0.42 mmol, 1 equiv) (from Example 2, step 3). (1R,3s,5S)-8-oxabicyclo[3.2.1]octan-3-amine (133.54 mg, 1.05 mmol, 2.5 equiv), diisopropylethylamine (DIEA) (271.40 mg, 2.10 mmol, 5 equiv), and dimethylsulfoxide (DMSO) (4 mL) at room temperature. The resulting mixture was stirred for overnight at 80° C. under nitrogen atmosphere. The reaction progress was monitored by LCMS. The residue was purified by reversed-phase flash chromatography Method C to provide 1-(2-methoxy-4-methylphenyl)-N-[(1R,3R,5S)-8-oxabicyclo[3.2.1]octan-3-yl]pyrido[3,4-d]pyridazin-4-amine (50 mg. 25.30% yield). LCMS (ES, m/z): RT=0.590 min, m/z=377 [M+1]⁺. ¹H NMR (400 MHz, DMSO-d₆) δ 9.74 (d, J=2.8 Hz, 2H), 7.58 (d, J=7.7 Hz, 1H), 7.35 (d, J=7.6 Hz, 1H), 7.23 (d, J=7.5 Hz, 1H), 7.03 (d, J=8.2 Hz, 2H), 3.70 (s, 1H), 3.69 (s, 1H), 3.66 (s, 3H), 2.47 (s, 1H), 2.43 (s, 3H), 2.10-1.90 (m, 2H), 1.96-1.85 (m, 4H), 1.82-1.74 (m, 2H). Stereochemistry rationally assigned based on chiral starting material and assumed stereochemical inversion in step 2.

Step 5. Into a 8 mL. vial was added 1-(2-methoxy-4-methylphenyl)-N-[(1R,5R)-8-oxabicyclo[3.2.1]octan-3-yl]pyrido[3,4-d]pyridazin-4-amine (50 mg. 0.13 mmol, 1 equiv), (ethylsulfanyl) sodium (EtSNa) (279 mg, 3.33 mmol. 25 equiv), and dimethylsulfoxide (DMSO) (2.5 mL) at room temperature. The resulting mixture was stirred for 2 h at 120° C. under nitrogen atmosphere. The reaction progress was monitored by LCMS. The residue was purified by reversed-phase flash chromatography Method C to provide the crude product (30 mg, 70% purity), which was further purified by Prep-HPLC Method K (12.1 mg. 25.0% yield) to provide 2-(4-(((1R,3s,5S)-8-oxabicyclo[3.2.1]octan-3-yl)amino)pyrido[3,4-d]pyridazin-1-yl)-5-methylphenol (Compound 2B*-r). LCMS: (ES, m/z): RT=0.588 min, m/z=363 [M+1]⁺. ¹H NMR (400 MHZ, DMSO-d₆) δ 9.74 (s, 1H), 9.59 (s, 1H), 8.84 (d, J=5.6 Hz, 1H), 7.56 (d, J=7.7 Hz, 1H), 7.31 (d, J=5.7 Hz, 1H), 7.20 (d, J=7.6 Hz, 1H), 6.85-6.74 (m, 2H), 4.83 (s, 1H), 4.44 (s, 2H), 2.34 (s, 3H), 2.11-1.95 (m, 2H), 1.97-1.81 (m, 4H), 1.78 (t, J=11.1 Hz, 2H).

Stereochemistry of Compound 2B*-r was rationally assigned based on chiral starting material and assumed stereochemical inversion in step 2.

### Example 4. 2-(4-(((1R,5S,8s)-3-oxabicyclo[3.2.1]octan-8-yl)amino)pyrido[3,4-d]pyridazin-1-yl)-5-chlorophenol (Compound 5A)

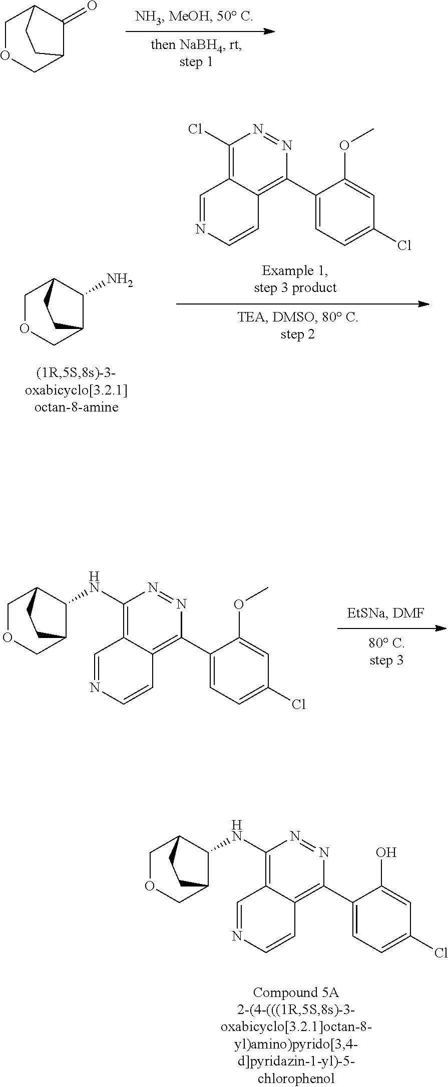 [SMILES: O=C1C2CCC1COC2 ; COc1cc(Cl)ccc1-c1nnc(Cl)c2cnccc12 ; N[C@H]1[C@@H]2CC[C@H]1COC2 ; COc1cc(Cl)ccc1-c1nnc(N[C@H]2[C@@H]3CC[C@H]2COC3)c2cnccc12 ; Oc1cc(Cl)ccc1-c1nnc(N[C@H]2[C@@H]3CC[C@H]2COC3)c2cnccc12]

Step 1. Into a 8 mL vial was added 3-oxabicyclo[3.2.1]octan-8-one (80.0 mg, 0.63 mmol, 1 equiv) and ammonia in methanol (MeOH) solution (7M) 3 mL at room temperature. The resulting mixture was stirred for 8 h at 50° C. The mixture was allowed to cool to room temperature. The reaction was monitored by LCMS. To the above mixture was added NaBH₄ (36.0 mg, 0.95 mmol, 1.5 equiv) dropwise for 10 min at room temperature. The resulting mixture was stirred for additional 2 h at room temperature. The reaction was monitored by LCMS. The reaction was quenched by water/ice (10 mL) at 0° C. The resulting mixture was extracted with ethyl acetate (EtOAc) (3×10 mL). The combined organic layers were washed with water (3×10 mL), dried over anhydrous Na₂SO₄. After filtration, the filtrate was concentrated under reduced pressure. This resulted in 3-oxabicyclo[3.2.1]octan-8-amine (50 mg. 56% yield). LCMS (ES, m/z): RT=0.74 min, m/z=128.0[M+1]+. ¹H NMR (400 MHZ. DMSO-d₆) δ 4.12 (m, 3H). 3.40 (d, J=2.3 Hz, 3H), 2.96 (t, J=4.6 Hz, 1H), 1.96-1.89 (m, 4H), 1.78 (d, J=2.9 Hz, 2H). Stereochemistry retroactively assigned to be (1R,5S,8s)-3-oxabicyclo[3.2.1]octan-8-amine based on absolute stereochemical determination of Compound 6A. See Example 5.

Step 2. Into a 20 mL vial was added (1R,5S,8s)-3-oxabicyclo[3.2.1]octan-8-amine (30.0 mg. 0.24 mmol, 1 equiv), triethylamine (TEA) (47.8 mg. 0.47 mmol, 2 equiv) and dimethylsulfoxide (DMSO) (2 mL) at room temperature. The resulting mixture was stirred for 8 h at 80° C. The reaction was monitored by LCMS. The resulting mixture was diluted with water (5 mL) and extracted with ethyl acetate (3×10 mL). The combined organic layers were washed with water (3×10 mL), dried over anhydrous Na₂SO₄. After filtration, the filtrate was concentrated under reduced pressure to provide a crude residue which was purified by reverse phase flash chromatography (acetonitrile: H₂O=3:1) to afford N-((1R,5S,8s)-3-oxabicyclo[3.2.1]octan-8-yl)-1-(4-chloro-2-methoxyphenyl)pyrido[3,4-d]pyridazin-4-amine (50.0 mg, 53% yield). LCMS (ES, m/z): RT=0.96 min, m/z=397.0 [M+1]+. Stereochemistry retroactively assigned based on absolute stereochemical determination of Compound 6A.

Step 3. Into a 8 mL sealed tube was added N-((1R,5S,8s)-3-oxabicyclo[3.2.1]octan-8-yl)-1-(4-chloro-2-methoxyphenyl)pyrido[3,4-d]pyridazin-4-amine (100 mg, 0.25 mmol, 1 equiv), (ethylsulfanyl) sodium (EtSNa) (423.86 mg. 5.04 mmol, 20 equiv) and dimethylformamide (1 mL) at room temperature. The resulting mixture was stirred for overnight at 80° C. The reaction was monitored by LCMS. The mixture was purified by reverse phase flash with the following conditions (acetonitrile: H₂O 1:3) to afford a crude residue (70 mg) which was purified by prep-HPLC Method P (RT=7.5 min) to provide 2-(4-(((1R,5S,8s)-3-oxabicyclo[3.2.1]octan-8-yl)amino)pyrido[3,4-d]pyridazin-1-yl)-5-chlorophenol (Compound 5A) (50.20 mg. 52% yield). LCMS (ES, m/z): RT=0.63 min, m/z=383.0 [M+1]+. ¹H NMR (400 MHZ. Methanol-d₄) δ 9.83 (s, 1H), 8.89 (d, J=5.7 Hz, 1H). 7.55 (d, J=5.8 Hz, 1H), 7.39 (d, J=7.9 Hz, 1H), 7.10-7.02 (m, 2H), 4.20 (d, J=11.6 Hz, 3H), 3.51 (d, J=10.7 Hz, 2H), 2.59 (s, 2H), 2.06-1.91 (m, 4H).

Stereochemistry of Compound 5A retroactively assigned based on absolute stereochemical determination of Compound 6A.

### Example 5. 2-(4-(((1R,5S,8s)-3-oxabicyclo[3.2.1]octan-8-yl)amino)pyrido[3,4-d]pyridazin-1-yl)-5-methylphenol (Compound 6A)

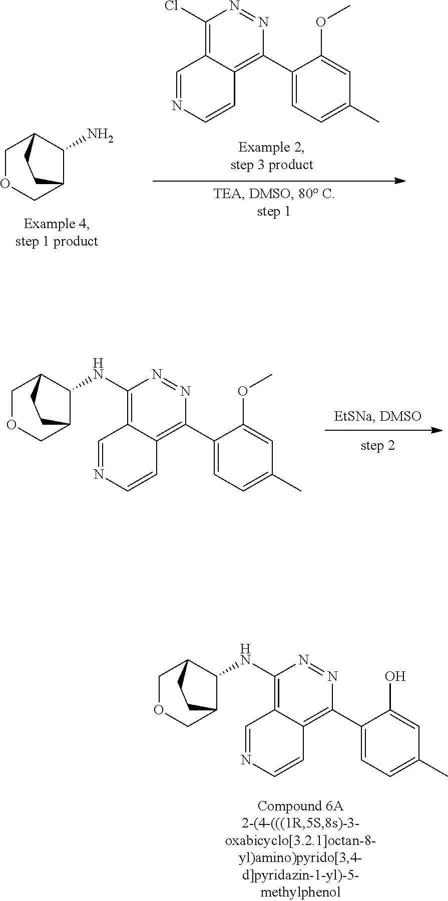 [SMILES: COc1cc(C)ccc1-c1nnc(Cl)c2cnccc12 ; N[C@H]1[C@@H]2CC[C@H]1COC2 ; COc1cc(C)ccc1-c1nnc(N[C@H]2[C@@H]3CC[C@H]2COC3)c2cnccc12 ; Cc1ccc(-c2nnc(N[C@H]3[C@@H]4CC[C@H]3COC4)c3cnccc23)c(O)c1]

Step 1. Into a & mL vial was added (1R,5S,8s)-3-oxabicyclo[3.2.1]octan-8-amine (from Example 4, step 1; stereochemistry retroactively assigned based on absolute stereochemical determination of Compound 6A) (20 mg. 0.16 mmol, 1 equiv), 4-chloro-1-(2-methoxy-4-methylphenyl)pyrido[3,4-d]pyridazine (from Example 2, step 3) (53.9 mg, 0.188 mmol, 1.2 equiv), triethylamine (TEA) (19.1 mg, 0.188 mmol, 1.2 equiv) and dimethylsulfoxide (DMSO) (1 mL) at room temperature. The resulting mixture was stirred for overnight at 80° C. The reaction was monitored by LCMS. The resulting mixture was diluted with water (5 mL). The resulting mixture was extracted with ethyl acetate (3×10 mL). The combined organic layers were washed with water (3×10 mL), dried over anhydrous Na₂SO₄. After filtration, the filtrate was concentrated under reduced pressure to provide a crude residue which was purified by reverse phase flash chromatography (acetonitrile: H₂O=3:1) to afford N-((1R,5S,8s)-3-oxabicyclo[3.2.1]octan-8-yl)-1-(2-methoxy-4-methylphenyl)pyrido[3,4-d]pyridazin-4-amine (75 mg, 17% yield).

Step 2. Into a 8 mL sealed tube were added N-(1R,5S,8s)-3-oxabicycle[3.2.1]octan-8-(1)-1-(2-methoxy-4-methylphenyl)pyrido[3,4-d]pyridazin-4-amine (50 mg. 0.133 mmol, 1 equiv), (ethylsulfanyl) sodium (EtSNa) (223 mg, 2.66 mmol. 20 equiv) and dimethylformamide (DMF) (1 mL) at room temperature. The resulting mixture was stirred for overnight at 100° C., and monitored by LCMS. After completion of the reaction, the residue was purified by reverse flash chromatography Method Q (RT=8.5 min) to provide 2-(4-(((1R,5S,8s)-3-oxabicyclo[3.2.1]octan-8-yl)amino)pyrido[3,4-d]pyridazin-1-yl)-5-methylphenol (Compound 6A) (13.6 mg. 28.1% yield). LCMS (ES, m/z): RT=1.32 min, m/z=363.0 [M+1]+. ¹H NMR (400 MHZ, DMSO-d₆) δ 9.94 (s, 1H), 9.62 (s, 1H), 8.88 (d, J=5.6 Hz, 1H), 7.75 (d, J=3.5 Hz, 1H), 7.35 (d, J=5.6 Hz, 1H), 7.21 (d, J=7.5 Hz, 1H), 6.85-6.77 (m, 2H), 4.10 (d, J=11.1 Hz, 3H), 3.37 (dd, J=11.6, 2.6 Hz, 3H), 2.47 (s, 1H). 2.34 (s, 3H), 1.89 (d, J=10.6 Hz, 2H), 1.85-1.77 (m, 2H).

Absolute stereochemistry of Compound 6A confirmed by X-ray crystallography.

### Example 6. 2-(4-((7-oxabicyclo[2.2.1]heptan-2-yl)amino)pyrido[3,4-d]pyridazin-1-yl)-5-chlorophenol (Compound 3); 2-(4-(((1R,4S)-7-oxabicyclo[2.2.1]heptan-2-yl)amino)pyrido[3,4-d]pyridazin-1-yl)-5-chlorophenol (Compound 3′*); 2-(4-(((1S,4R)-7-oxabicyclo[2.2.1]heptan-2-yl)amino)pyrido[3,4-d]pyridazin-1-yl)-5-chlorophenol (Compound 3″*); 2-(4-(((1R,2R,4S)-7-oxabicyclo[2.2.1]heptan-2-yl)amino)pyrido[3,4-d]pyridazin-1-yl)-5-chlorophenol (Compound 3A*); 2-(4-(((1R,2S,4S)-7-oxabicyclo[2.2.1]heptan-2-yl)amino)pyrido[3,4-d]pyridazin-1-yl)-5-chlorophenol (Compound 3B*); 2-(4-(((1S,2R,4R)-7-oxabicyclo[2.2.1]heptan-2-yl)amino)pyrido[3,4-d]pyridazin-1-yl)-5-chlorophenol (Compound 3C*); 2-(4-(((1S,2S,4R)-7-oxabicyclo[2.2.1]heptan-2-yl)amino)pyrido[3,4-d]pyridazin-1-yl)-5-chlorophenol (Compound 3D*)

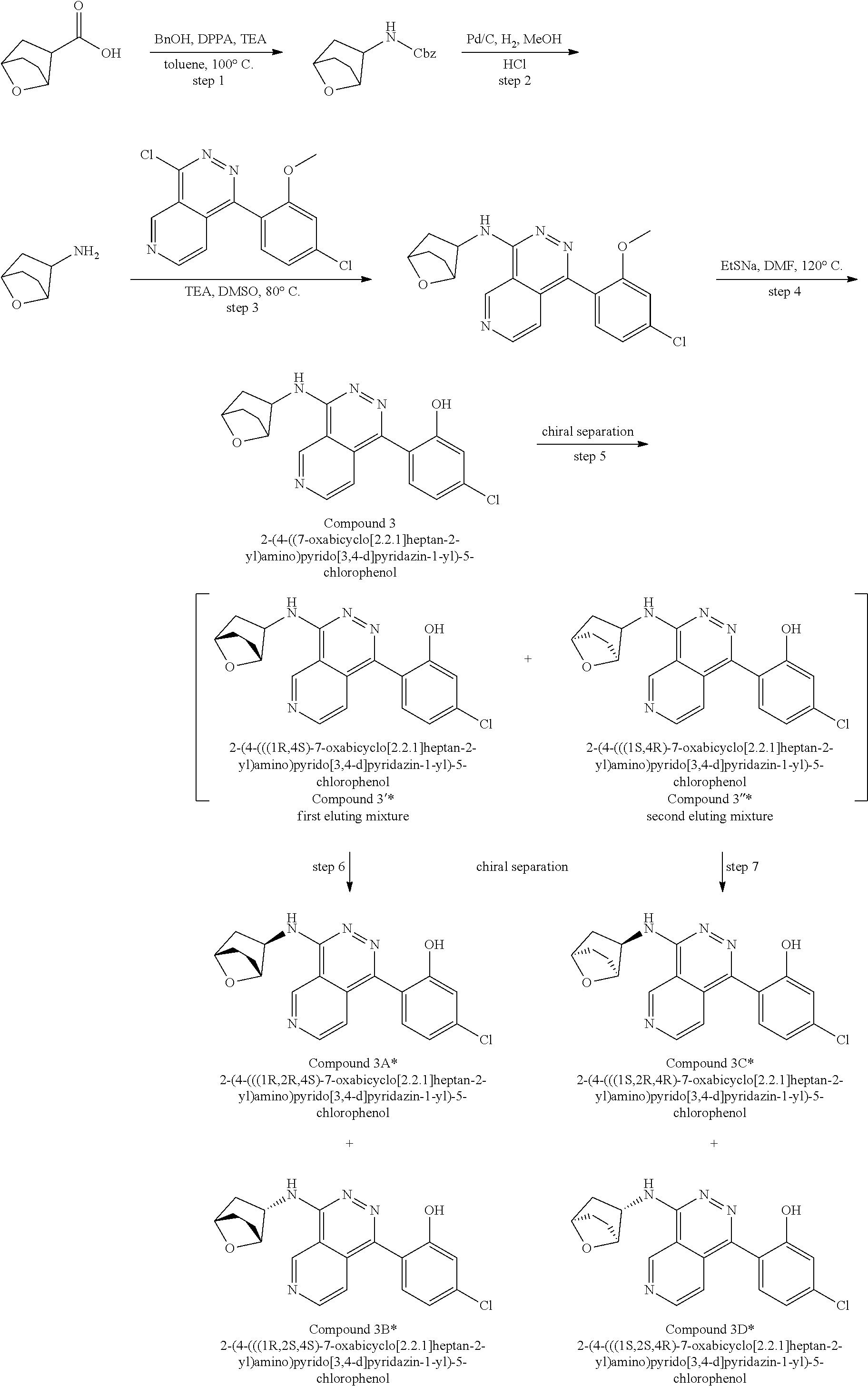

Step 1. Into a 20 mL vial was added 7-oxabicyclo[2.2.1]heptane-2-carboxylic acid (700 mg, 4.92 mmol, 1 equiv), benzyl alcohol (1 mL), diphenylphosphoryl azide (DPPA) (2710 mg, 9.840 mmol, 2 equiv), and toluene (10 mL) at room temperature. The resulting mixture was stirred for 2 h at 100° C. under nitrogen atmosphere. The reaction progress was monitored by LCMS, and the resulting reaction mixture was concentrated under reduced pressure to provide a residue which was purified by reverse flash chromatography Method C to afford benzyl N-{7-oxabicyclo[2.2.1]heptan-2-yl}carbamate (1.1 g, 90% yield). LCMS (ES,m/z): RT=0.801 min, m/z=248 [M+1]+. Bn=benzyl; Cbz=carbobenzyloxy.

Step 2. Into a 250 mL round-bottom flask was added benzyl N-{7-oxabicyclo[2.2.1]heptan-2-yl}carbamate (1 g, 4.04 mmol, 1 equiv), Pd/C (998 mg. 9.38 mmol, 2.32 equiv) and methanol (McOH) (50 mL) at room temperature. The resulting mixture was stirred for 2 h at room temperature under hydrogen atmosphere. The resulting mixture was then filtered, the filter cake was washed with MeOH (2×10 mL), and the filtrate concentrated under reduced pressure to afford 7-oxabicyclo[2.2.1]heptan-2-amine (300 mg). LCMS (ES, m/z): RT=0.158 min, m/z=114 [M+1]+.

Step 3. Into a 20 mL vial was added 7-oxabicyclo[2.2.1]heptan-2-amine (200 mg, 1.76 mmol, 1 equiv), 4-chloro-1-(4-chloro-2-methoxyphenyl)pyrido[3,4-d]pyridazine (270.5 mg, 0.88 mmol, 0.5 equiv), triethylamine (TEA) (888.8 mg, 8.8 mmol, 5 equiv), and dimethylsulfoxide (DMSO) (5 mL) at room temperature. The resulting mixture was stirred overnight at 80° C., and the reaction progress was monitored by LCMS. The reaction was added water (10 mL) and ethyl acetate (EtOAc) (10 mL), and the reaction mixture was extracted with EtOAc (3×10 mL). The combined organic layers were washed with H₂O (1×20 mL), dried over anhydrous Na₂SO₄, filtered, and the filtrate was concentrated under reduced pressure. The residue was purified by reverse flash chromatography Method C and concentrated to provide 1-(4-chloro-2-methoxyphenyl)-N-{7-oxabicyclo[2.2.1]heptan-2-yl}pyrido[3,4-d]pyridazin-4-amine (200 mg, 29.6% yield). LCMS: (ES,m/z): RT=1.074 min, m/z=383 [M+1]+.

Step 4. Into a 20 mL vial was added 1-(4-chloro-2-methoxyphenyl)-N-{7-oxabicyclo[2.2.1]heptan-2-yl}pyrido[3,4-d]pyridazin-4-amine (200 mg, 0.52 mmol, 1 equiv), (ethylsulfanyl) sodium (EtSNa) (439 mg. 5.22 mmol, 10 equiv), and dimethylsulfoxide (DMSO) (5 mL) at room temperature. The resulting mixture was stirred for 2 h at 100° C., and the reaction progress was monitored by LCMS. The reaction was added water (10 mL) and ethyl acetate (EtOAc) (10 mL), and the reaction mixture was extracted with EtOAc (3×10 mL). The combined organic layers were washed with H₂O (1×20 mL), dried over anhydrous Na—SO₄, filtered, and the filtrate was concentrated under reduced pressure. The residue was purified by reverse flash chromatography Method D to provide 2-(4-((7-oxabicyclo[2.2.1]heptan-2-yl)amino)pyrido[3,4-d]pyridazin-1-yl)-5-chlorophenol (90 mg, 96% purity) as a mixture of stereoisomers.

Step 5. The mixture product (90 mg. 96% purity) was purified by Chiral-Prep-HPLC Method E. and the resulting mixture was concentrated under reduced pressure to afford the first eluting mixture (100 mg, 98% purity) as 2-(4-(((1R,4S)-7-oxabicyclo[2.2.1]heptan-2-yl)amino)pyrido[3,4-d]pyridazin-1-yl)-5-chlorophenol (Compound 3′*) and the second eluting mixture (10 mg. 99.6% purity) as 2-(4-(((1S,4R)-7-oxabicyclo[2.2.1]heptan-2-yl)amino)pyrido[3,4-d]pyridazin-1-yl)-5-chlorophenol (Compound 3″*). Stereochemistry was arbitrarily assigned.

Step 6. The first eluting mixture (100 mg, 98% purity) was purified by Chiral-Prep-HPLC Method F to provide 2-(4-(((1R,2R,4S)-7-oxabicyclo[2.2.1]heptan-2-yl)amino)pyrido[3,4-d]pyridazin-1-$1)-5-chlorophenol (Compound 3A*) as the first eluting peak (28.8 mg, 15% yield: RT (min)=7.4) and 2-(4-(((1R,2S,4S)-7-oxabicyclo[2.2.1]heptan-2-yl)amino)pyrido[3,4-d]pyridazin-1-yl)-5-chlorophenol (Compound 3B*) as the second eluting peak (31.0 mg, 16% yield: RT (min)=11.53). Stereochemistry of Compound 3A* and Compound 3B* were arbitrarily assigned.

Compound 3A*: LCMS (ES,m/z): RT=1.213 min, m/z=369 [M+1]+, ¹H NMR (400 MHZ, Methanol-d₄) δ 10.05 (d, J=1.1 Hz, 1H), 9.12-9.08 (m, 1H). 7.70-7.65 (m, 1H), 7.43-7.38 (m, 1H), 7.19-7.08 (m, 2H), 5.04-5.00 (m, 1H), 4.79-4.70 (m, 1H), 4.41-4.35 (m. 1H), 2.58-2.39 (m, 1H), 2.03-1.97 (m, 1H), 1.96-1.80 (m, 3H), 1.80-1.65 (m, 1H).

Compound 3B*: LCMS (ES,m/z): RT=1.216 min, m/z=369 [M+1]+: 1H NMR (400 MHz, Methanol-d₄) δ 10.04 (s, 1H), 9.12 (d, J=5.5 Hz, 1H), 7.70-7.65 (m, 1H), 7.43 (d, J=8.1 Hz, 1H). 7.21-7.04 (m, 2H), 5.04-5.00 (m, 1H), 4.79-4.71 (m, 1H). 4.41-4.35 (m, 1H), 2.44-2.40 (m, 1H). 2.10-1.98-1.94 (m, 1H), 1.91-1.80 (m, 3H), 1.73-1.67 (m, 1H).

Step 7. The second eluting mixture (10 mg, 99% purity) was purified by Chiral-Prep-HPLC Method G to provide 2-(4-(((1S,2R,4R)-7-oxabicyclo[2.2.1]heptan-2-yl)amino)pyrido[3,4-d]pyridazin-1-yl)-5-chlorophenol (Compound 3C*) as the first eluting peak (1.4 mg, 0.73% yield; RT (min)=7.18) and 2-(4-(((1S,2S,4R)-7-oxabicyclo[2.2.1]heptan-2-yl)amino)pyrido[3,4-d]pyridazin-1-yl)-5-chlorophenol (Compound 3D*) as the second eluting peak (1.2 mg, 0.62% yield; RT (min)=9.6). Stereochemistry of Compound 3C* and Compound 3D* were arbitrarily assigned.

Compound 3C*: LCMS (ES,m/z): RT=1.202 min, m/z=369 [M+1]+; 1H NMR (400 MHZ, Methanol-d₆) δ 9.75 (s, 1H), 8.88 (d, J=5.7 Hz, 1H), 7.54-7.50 (m, 1H), 7.39 (d, J=7.9 Hz, 1H), 7.17-6.96 (m, 2H), 4.81-4.61 (m, 2H), 4.43-4.38 (m, 1H), 2.21-2.14 (m, 1H), 2.07-1.93 (m, 1H), 1.83-1.68 (m, 3H), 1.68-1.55 (m, 1H), 1.45-1.22 (m, 1H).

Compound 3D*: LCMS (ES,m/z): RT=1.199 min, m/z=369 [M+1]+: 1H NMR (400 MHZ, Methanol-d₄) δ 9.73 (s, 1H), 8.87 (d, J=5.7 Hz, 1H), 7.53 (d, J=5.6 Hz, 1H), 7.39 (d, J=7.9 Hz, 1H), 7.06 (d, J=10.4 Hz, 2H), 4.79-4.68 (m, 2H), 4.44-4.38 (m, 1H), 2.20-2.14 (m, 8.0 Hz, 1H), 1.97 (d, J=13.1 Hz, 1H), 1.75-1.69 (m, 3H), 1.68-1.55 (m, 1H), 1.32-1.28 (m, 1H).

Compounds provided in the below Table B were synthesized or may be synthesized following the above General Synthetic Protocol A and Protocol A Examples. Dashed lines (--) indicates that no data is available.

TABLE B
Protocol A Compounds

| | | LCMS | | |
|---|---|---|---|---|
| | | (m/z) | | |
| # | Compound | [M + 1]⁺ | ¹HNMR | Conditions |
| 1 | 2-(4-((8- | — | — | Compound 1B*-r may |
| | oxabicyclo[3.2.1]octan-3- | | | be synthesized following |
| | yl)amino)pyrido[3,4- | | | Protocol A, using 4- |
| | d]pyridazin-1-yl)-5- | | | chloro-1-(4-chloro-2- |
| | chlorophenol | | | methoxyphenyl)pyrido[3,4- |
| 1B*-r | 2-(4-(((1R,3s,5S)-8- | — | — | d]pyridazine of |
| | oxabicyclo[3.2.1]octan-3- | | | Example 1 and |
| | yl)amino)pyrido[3,4- | | | (1R,3s,5S)-8- |
| | d]pyridazin-1-yl)-5- | | | oxabicyclo[3.2.1]octan- |
| | chlorophenol | | | 3-amine of Example 3 |
| | | | | followed by |
| | | | | deprotection. |
| | | | | Stereochemistry of |
| | | | | Compound 1B*-r may |
| | | | | be rationally assigned |
| | | | | based on use of the |
| | | | | chiral amine prepared as |
| | | | | set forth in Example 3. |
| 2 | 2-(4-((8- | — | — | Compound 2 may be |
| | oxabicyclo[3.2.1]octan-3- | | | synthesized following |
| | yl)amino)pyrido[3,4- | | | Example 2 step 4 using |
| | d]pyridazin-1-yl)-5- | | | 8- |
| | methylphenol | | | oxabicyclo[3.2.1]octan- |
| | | | | 3-amine. |
| 5 | 2-(4-((3- | — | — | Compound 5B may be |
| | oxabicyclo[3.2.1]octan-8- | | | synthesized following |
| | yl)amino)pyrido[3,4- | | | Protocol A and Example |
| | d]pyridazin-1-yl)-5- | | | 4 using (1R,5S,8r)-3- |
| | chlorophenol | | | oxabicyclo[3.2.1]octan- |
| 5B | 2-(4-(((1R,5S,8r)-3- | — | — | 8-amine as the step 4 |
| | oxabicyclo[3.2.1]octan-8- | | | amine (i) reagent. |
| | yl)amino)pyrido[3,4- | | | (1R,5S,8r)-3- |
| | d]pyridazin-1-yl)-5- | | | oxabicyclo[3.2.1]octan- |
| | chlorophenol | | | 8-amine may be |
| | | | | synthesized from |
| | | | | mesylation of |
| | | | | (1R,5S,8s)-3- |
| | | | | oxabicyclo[3.2.1]octan- |
| | | | | 8-ol, followed by NaN₃, |
| | | | | and reduction, similar to |
| | | | | Example 3, steps 1-3. |
| | | | | Stereochemistry may be |
| | | | | retroactively assigned |
| | | | | based on absolute |
| | | | | stereochemical |
| | | | | determination of |
| | | | | Compound 6A. |
| 6 | 2-(4-((3- | — | — | Compound 6B may be |
| | oxabicyclo[3.2.1]octan-8- | | | synthesized following |
| | yl)amino)pyrido[3,4- | | | Protocol A and Example |
| | d]pyridazin-1-yl)-5- | | | 4 using (1R,5S,8r)-3- |
| | methylphenol | | | oxabicyclo[3.2.1]octan- |
| 6B | 2-(4-(((1R,5S,8r)-3- | — | — | 8-amine as the step 4 |
| | oxabicyclo[3.2.1]octan-8- | | | amine (i) reagent. |
| | yl)amino)pyrido[3,4- | | | Stereochemistry may be |
| | d]pyridazin-1-yl)-5- | | | retroactively assigned |
| | methylphenol | | | based on absolute |
| | | | | stereochemical |
| | | | | determination of |
| | | | | Compound 6A. |
| 7 | 2-(4-((7- | — | — | Compounds 7A*-7D* |
| | oxabicyclo[2.2.1]heptan-2- | | | were synthesized |
| | yl)amino)pyrido[3,4-in-1-yl)- | | | following Protocol A |
| | 5-methylphenol | | | and Example 6 using 7- |
| 7′* | 2-(4-(((1R,4S)-7- | — | — | oxabicyclo [2.2.1] |
| | oxabicyclo[2.2.1]heptan-2- | | | heptan-2-amine as the |
| | yl)amino)pyrido[3,4- | | | step 4 amine (i) reagent. |
| | d]pyridazin-1-yl)-5- | | | Stereochemistry |
| | methylphenol | | | arbitrarily assigned. |
| 7″* | 2-(4-(((1S,4R)-7- | — | — | Method R |
| | oxabicyclo[2.2.1]heptan-2- | | | (7D* RT = 3.45 min; |
| | yl)amino)pyrido[3,4- | | | 7B* RT = 4.20 min) |
| | d]pyridazin-1-yl)-5- | | | Method S |
| | methylphenol | | | (7C* RT = 3.09 min; |
| 7A* | 2-(4-(((1R,2R,4S)-7- | 349.10 | ¹H NMR (400 MHz, | 7A* RT = 8.5 min) |
| | oxabicyclo[2.2.1]heptan-2- | | Methanol-d₄) δ 9.72 (d, J = | |
| | yl)amino)pyrido[3,4- | | 1.0 Hz, 1H), 8.85 (d, J = | |
| | d]pyridazin-1-yl)-5- | | 5.7 Hz, 1H), 7.62-7.53 | |
| | methylphenol | | (m, 1H), 7.29 (d, J = 7.7 | |
| | | | Hz, 1H), 6.94-6.81 (m, | |
| | | | 2H), 4.76-4.68 (m, 2H), | |
| | | | 4.44-4.42 (m, 1H), 2.40 | |
| | | | (s, 3H), 2.27-2.21 (m, | |
| | | | 1H), 1.96 (d, J = 13.0 Hz, | |
| | | | 1H), 1.83-1.66 (m, 3H), | |
| | | | 1.64-1.55 (m, 1H). | |
| 7B* | 2-(4-(((1R,2S,4S)-7- | 349.10 | ¹H NMR (400 MHz, | |
| | oxabicyclo[2.2.1]heptan-2- | | Methanol-d₄) δ 9.85 (d, J = | |
| | yl)amino)pyrido[3,4- | | 1.0 Hz, 1H), 8.94 (d, J = | |
| | d]pyridazin-1-yl)-5- | | 5.6 Hz, 1H), 7.65-7.62 | |
| | methylphenol | | (m, 1H), 7.29 (d, J = 7.7 | |
| | | | Hz, 1H), 6.94-6.84 (m, | |
| | | | 2H), 5.16-5.13 (m, 1H), | |
| | | | 4.71-4.63 (m, 1H), 4.50- | |
| | | | 4.44 (m, 1H), 2.41 (s, | |
| | | | 4H), 2.06-1.96 (m, 1H), | |
| | | | 1.86-1.71 (m, 3H), 1.68- | |
| | | | 1.61 (m, 1H). | |
| 7C* | 2-(4-(((1S,2R,4R)-7- | 349.10 | ¹H NMR (400 MHz, | |
| | oxabicyclo[2.2.1]heptan-2- | | Methanol-d₄) δ 9.71 (d, J = | |
| | yl)amino)pyrido[3,4- | | 1.0 Hz, 1H), 8.85 (d, J = | |
| | d]pyridazin-1-yl)-5- | | 5.6 Hz, 1H), 7.58-7.54 | |
| | methylphenol | | (m, 1H), 7.28 (d, J = 7.6 | |
| | | | Hz, 1H), 6.93-6.81 (m, | |
| | | | 2H), 4.75-4.67 (m, 2H), | |
| | | | 4.48-4.41 (m, 1H), 2.40 | |
| | | | (s, 3H), 2.27-2.21 (m, | |
| | | | 1H), 1.96 (d. J = 12.5 Hz, | |
| | | | 1H), 1.79-1.69 (m, 3H), | |
| | | | 1.66-1.54 (m, 1H) | |
| 7D* | 2-(4-(((1S,2S,4R)-7- | 349.10 | ¹H NMR (400 MHz, | |
| | oxabicyclo[2.2.1]heptan-2- | | Methanol-d₄) δ 9.82 (d, J = | |
| | yl)amino)pyrido[3,4- | | 1.0 Hz, 1H), 8.90 (d, J = | |
| | d]pyridazin-1-yl)-5- | | 5.7 Hz, 1H), 7.61-7.59 | |
| | methylphenol | | (m, 1H), 7.29 (d, J = 7.7 | |
| | | | Hz. 1H), 6.96-6.83 (m, | |
| | | | 2H), 5.18-5.12 (m, 1H), | |
| | | | 4.68-4.62 (m, 1H), 4.51- | |
| | | | 4.46 (m, 1H), 2.41 (s, | |
| | | | 4H), 2.05-2.01 (m, 1H), | |
| | | | 1.86-1.77 (m, 2H), 1.75- | |
| | | | 1.71 (m, 1H), 1.70 - | |
| | | | 1.54 (m, 1H). | |
| 8 | 2-(4-((2- | — | — | Compounds 8A*-8B* |
| | oxabicyclo[2.2.1]heptan-5- | | | were synthesized |
| | yl)amino)pyrido[3,4- | | | following Protocol A |
| | d]pyridazin-1-yl)-5- | | | and Examples 1-6 using |
| | chlorophenol | | | 2-oxabicyclo |
| 8A* | 2-(4-(((1S,4R,5R)-2- | 369.10 | ¹H NMR (400 MHz, | [2.2.1]heptan-5-amine as |
| | oxabicyclo[2.2.1]heptan-5- | | Methanol-d₄) δ 9.89 (s, | the step 4 amine (i) |
| | yl)amino)pyrido[3,4- | | 1H), 9.00 (d, J = 5.6 Hz, | reagent. |
| | d]pyridazin-1-yl)-5- | | 1H), 7.65-7.54 (m, 2H), | Stereochemistry |
| | chlorophenol | | 7.37 (d, J = 7.8 Hz, 1H), | arbitrarily assigned. |
| | | | 7.31 (s, 1H), 4.77 (d, J = | Method T |
| | | | 5.7 Hz, 2H), 4.36 (m, J = | (8A* RT = 0.84 min; |
| | | | 7.9. 3.2 Hz, 1H), 2.27 (m, | 8B* RT = 0.84 min) |
| | | | J = 12.9, 7.9 Hz. 1H), 2.05 | |
| | | | (s, 1H), 1.88-1.68 (m, | |
| | | | 3H), 1.63 (m, J = 11.3, 7.2 | |
| | | | Hz, 1H). | |
| 8B* | 2-(4-(((1R,4S,5S)-2- | 369.10 | ¹H NMR (400 MHz, | |
| | oxabicyclo[2.2.1]heptan-5- | | Methanol-d₄) δ 9.89 (s, | |
| | yl)amino)pyrido[3,4- | | 1H), 9.00 (d, J = 5.6 Hz, | |
| | d]pyridazin-1-yl)-5- | | 1H), 7.65-7.54 (m, 2H), | |
| | chlorophenol | | 7.37 (d, J = 7.8 Hz, 1H), | |
| | | | 7.31 (s, 1H), 4.77 (d, J = | |
| | | | 5.7 Hz, 2H), 4.36 (m, J = | |
| | | | 7.9, 3.2 Hz, 1H), 2.27 (m, | |
| | | | J = 12.9, 7.9 Hz, 1H), 2.05 | |
| | | | (s, 1H), 1.88-1.68 (m, | |
| | | | 3H), 1.63 (m, J = 11.3, 7.2 | |
| | | | Hz, 1H). | |
| 9 | 2-(4-((2- | — | — | Compounds 9A*-9B* |
| | oxabicyclo[2.2.1]heptan-4- | | | were synthesized |
| | yl)amino)pyrido[3,4- | | | following Protocol A |
| | d]pyridazin-1-yl)-5- | | | and Examples 1-6 using |
| | chlorophenol | | | 2-oxabicyclo |
| 9A* | 2-(4-(((4R)-2- | 369.05 | ¹H NMR (400 MHz, | [2.2.1]heptan-4-amine as |
| | oxabicyclo[2.2.1]heptan-4- | | DMSO-d₆) δ 10.25 (s, | the step 4 amine (i) |
| | yl)amino)pyrido[3,4- | | 1H), 9.17 (d, J = 0.9 Hz, | reagent. |
| | d]pyridazin-1-yl)-5- | | 1H), 8.75 (d, J = 5.5 Hz, | Stereochemistry |
| | chlorophenol | | 1H), 7.33-7.23 (m, 1H), | arbitrarily assigned. |
| | | | 7.00 (d, J = 7.4 Hz, 3H), | Method U |
| | | | 4.63 (d, J = 3,6 Hz, 1H), | (9A* RT = 1.62 min; |
| | | | 4.40-4.32 (m, 1H), 4.14 | 9B* RT = 2.22 min) |
| | | | (d, J = 11.0 Hz, 1H), 4.06 | |
| | | | (d, J = 11.0 Hz, 1H), 2.14- | |
| | | | 2.01 (m, 2H), 1.99- | |
| | | | 1.92 (m, 1H), 1.90-1.77 | |
| | | | (m, 1H), 1.79-1.70 (m, | |
| | | | 1H), 1.69-1.58 (m, 1H). | |
| 9B* | 2-(4-(((4S)-2- | 369.05 | ¹H NMR (400 MHz, | |
| | oxabicyclo[2.2.1]heptan-4- | | Methanol-d₄) δ 9.37 (d, J = | |
| | yl)amino)pyrido[3,4- | | 0.9 Hz. 1H), 8.77 (d, J = | |
| | d]pyridazin-1-yl)-5- | | 5.5 Hz, 1H), 7.34-7.26 | |
| | chlorophenol | | (m, 1H), 7.29-7.20 (m, | |
| | | | 1H), 7.01 (d, J = 7.8 Hz, | |
| | | | 2H), 4.59-4.50 (m, 1H), | |
| | | | 4.34 (d. J = 11.2 Hz, 1H), | |
| | | | 4.25 (d, J = 11.2 Hz, 1H), | |
| | | | 2.35-2.20 (m, 2H), 2.16- | |
| | | | 1.98 (m, 2H), 1.99- | |
| | | | 1.90 (m, 1H), 1.88-1.76 | |
| | | | (m, 1H). | |

### Protocol B Examples

### Example 7. 2-(4-(((1R,3r,5S)-8-oxabicyclo[3.2.1]octan-3-yl)amino)pyrido[3,4-d]pyridazin-1-yl)-5-(trifluoromethyl) phenol (Compound 4A*-r)

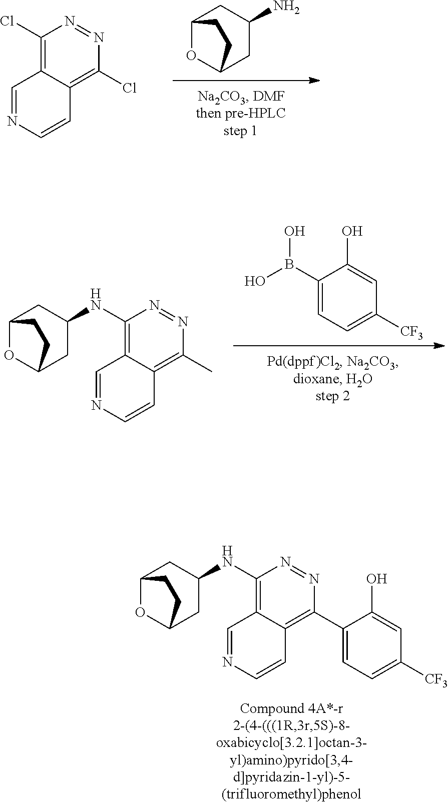

Step 1. Into a 40 mL vial was added 1,4-dichloropyrido[3,4-d]pyridazine (1.60 g, 7.99 mmol, 1 equiv), commercially available (1R,3r,5S)-8-oxabicyclo[3.2.1]octan-3-amine (813.9 mg, 6.39 mmol. 0.8 equiv). Na₂CO₃ (2540 mg. 24 mmol, 3 equiv), and dimethylformamide (DMF) (16 mL). The resulting mixture was stirred for 1 h at 120° C., and monitored by LCMS. The resulting mixture was then filtered, the filter cake was washed with DMF (3×5 mL), and the filtrate concentrated under reduced pressure to provide a crude residue which was purified by Prep-HPLC Method L to provide N-((1R,3r,5S)-8-oxabicyclo[3.2.1]octan-3-yl)-1-chloropyrido[3,4-d]pyridazin-4-amine (600 mg, 23.2% yield). LCMS: (ES, m/z): RT=0.51 min, m/z=291.0 [M+H]⁺. ¹H NMR (300 MHz, DMSO-d₆) δ 9.75 (s, 1H), 8.29-8.08 (m, 1H), 7.88 (d, J=5.6 Hz, 1H), 7.57 (d, J=3.7 Hz, 1H), 4.27 (d, J=28.8 Hz, 3H), 2.22-2.05 (m, 4H). 1.98 (d, J=14.6 Hz, 2H), 1.90-1.73 (m, 2H). Minor regioisomer (addition of amine at other chloro site) was not detected. Stereochemistry of product was rationally assigned based on use of chiral starting material.

Step 2. Into a 8 mL vial was added N-((1R,3r,5S)-8-oxabicyclo[3.2.1]octan-3-yl)-1-chloropyrido[3,4-d]pyridazin-4-amine (150 mg, 0.51 mmol, 1 equiv), 2-hydroxy-4-(trifluoromethyl)phenylboronic acid (266 mg, 1.29 mmol, 2.5 equiv), [1,1′-bis(diphenylphosphino) ferrocene]dichloro palladium (II) dichloromethane complex ((Pd(dppf)Cl₂CH₂Cl₂) (113.25 mg, 0.15 mmol, 0.3 equiv), Na₂CO₃ (164.04 mg, 1.54 mmol, 3 equiv), dioxane (1.50 mL), and H₂O (0.30 mL). The resulting mixture was stirred for 2 h at 80° C. under nitrogen atmosphere and monitored by LCMS. The reaction was then quenched with H₂O (10 mL) at room temperature, and the resulting mixture was extracted with Ethyl acetate (EtOAc) (3×10 mL). The combined organic layers were washed with brine (1×10 mL), and dried over anhydrous Na₂SO₄. After filtration, the filtrate was concentrated under reduced pressure to provide a residue which was purified by reversed-phase flash chromatography Method N, to provide the crude 2-(4-(((1R,3r,8S)-8-oxabicyclo[3.2.1]octan-3-yl)amino)pyrido[3,4-d]pyridazin-1-yl)-5-(trifluoromethyl) phenol (Compound 4A*-r) (110 mg), which was further purified by Prep-HPLC Method O (59.1 mg, 27.3% yield). LCMS (ES, m/z): RT=1.19 min, m/z=417.0 [M+H]⁺. ¹H NMR (400 MHz, DMSO-d₆) δ 10.46 (d, J=16.7 Hz, 1H), 9.75 (s, 1H), 8.89 (d, J=5.6 Hz, 1H), 7.58 (d, J=7.8 Hz, 1H), 7.36-7.26 (m, 3H), 4.38 (d, J=13.2 Hz, 3H), 2.28-2.13 (m, 4H), 2.10-2.00 (m, 2H), 1.96-1.84 (m, 2H).

Stereochemistry of Compound 4A*-r was rationally assigned based on use of chiral starting material.

### Example 8. 6-(4-(((1R,5S,8s)-3-oxabicyclo[3.2.1]octan-8-yl)amino)pyrido[3,4-d]pyridazin-1-yl)-2-fluoro-3-methylphenol (Compound 15A) and 6-(1-(((1R,5S,8s)-3-oxabicyclo[3.2.1]octan-8-yl)amino)pyrido[3,4-d]pyridazin-4-yl)-2-fluoro-3-methylphenol (Compound 15A-regioisomer)

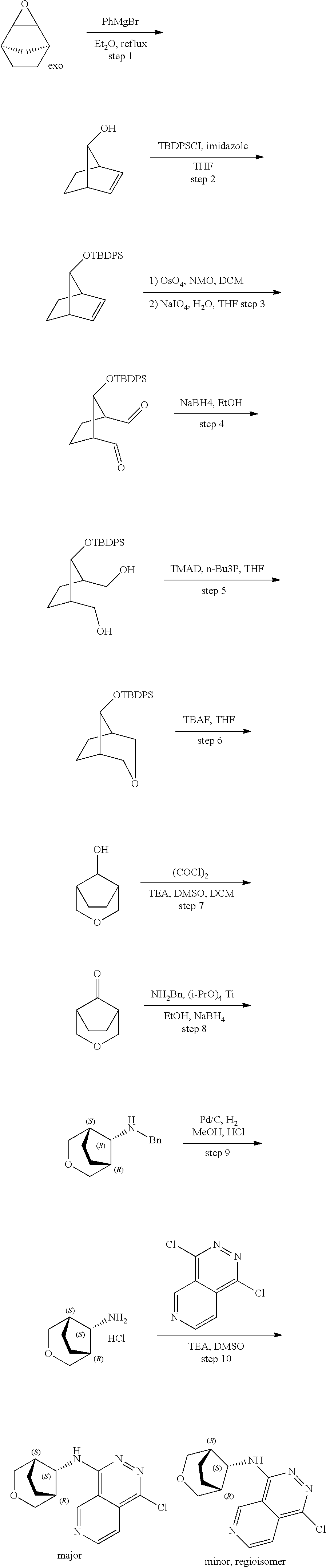

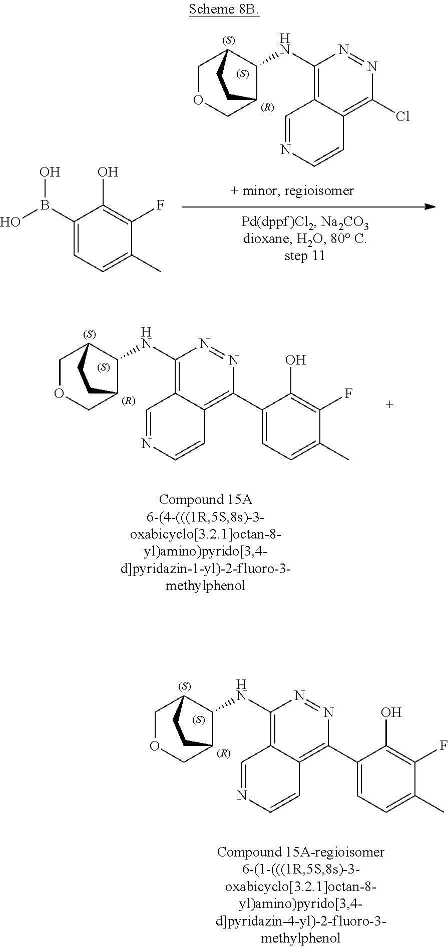

Step 1. To a stirred solution of exo-2,3-epoxynorbornane (25.0 g, 227 mmol, 1 equiv) in Et-O (500 mL) was added phenylmagnesium bromide solution (PhMgBr) (3M in diethylether (Et-O), 113 mL, 340 mmol, 1.5 equiv) dropwise at 0° C. under nitrogen atmosphere. The resulting mixture was stirred for overnight at 35° C. under nitrogen atmosphere. The reaction was monitored by GCMS. The reaction was quenched by the addition of water (500 mL) at room temperature. The mixture was adjusted to pH=6 with HCl (6M). The resulting mixture was extracted with diethylether (Et₂O) (3×400 mL). The combined organic lavers were washed with water (2×200 mL), dried over anhydrous Na₂SO₄. After filtration, the filtrate was concentrated under reduced pressure to provide bicyclo[2.2.1]hept-2-en-7-ol (24 g, 96% yield). GCMS: (ES, m/z): RT=3.9 min, m/z=110.1 [M].

Step 2. Into a 500 mL 3-necked round-bottom flask were added bicyclo[2.2.1]hept-2-en-7-ol (24.0 g, 218 mmol, 1 equiv), tetrahydrofuran (THF) (250 mL), imidazole (44.5 g, 654 mmol, 3 equiv), and tertbutylchlorodiphenylsilane (TBDPSCl) (120 g, 436 mmol, 2 equiv) at room temperature. The resulting mixture was stirred for 2 h at 60° C. under nitrogen atmosphere. The reaction was monitored by TLC. The reaction was quenched by the addition of water (400 mL) at room temperature. The aqueous layer was extracted with ethyl acetate (EtOAc) (3×500 mL) and the volatiles were removed under reduced pressure. The residue was purified by silica gel column chromatography, eluting with petroleum ether/ethyl acetate (12:1) to afford bicyclo[2.2.1]hept-2-en-7-yloxy}(tert-butyl)diphenylsilane (40 g. 53% yield). TLC: petroleum ether/ethyl acetate=10:1, Rf=0.4.

Step 3. To a stirred solution of bicyclo[2.2.1]hept-2-en-7-yloxy}(tert-butyl)diphenylsilane (40.0 g. 115 mmol, 1 equiv) and N-methylmorpholine N-oxide (NMO) (40.3 g. 344 mmol, 3 equiv) in dichloromethane (DCM) (200 mL) was added OsO₄ (5.83 g. 23.0 mmol, 0.20 equiv) in portions at room temperature under nitrogen atmosphere. The resulting mixture was stirred overnight at room temperature under nitrogen atmosphere. The reaction was monitored by LCMS. The reaction was quenched with saturated aqueous Na₂S₂O₃ (100 mL), and the mixture was stirred for 1 h. The aqueous layer was extracted with ethyl acetate (EtOAc) (3×800 mL). The resulting mixture was concentrated under reduced pressure. The residue was used in the next step without further purification. To an ice cold solution of the crude materials in tetrahydrofuran (THF) (400 mL), H₂O (400 mL) was added NalO₄ (73.7 g. 344 mmol. 3 equiv), and the reaction mixture was stirred at room temperature for 1 h. The reaction was monitored by TLC. The aqueous layer was extracted with EtOAc (3×500 mL). The residue was purified by silica gel column chromatography, eluting with petroleum ether/ethyl acetate (1:1) to afford 2-[(tert-butyldiphenylsilyl)oxy]cyclopentane-1,3-dicarbaldehyde (20 g. 46% yield). TLC: petroleum ether/ethyl acetate=1:1, Rf=0.2.

Step 4. To a stirred solution of 2-[(tert-butyldiphenylsilyl)oxy]cyclopentane-1,3-dicarbaldehyde (20 g. 52.6 mmol, 1 equiv) in tetrahydrofuran (THF) (1000 mL) was added NaBH: (5.96 g. 158 mmol. 3 equiv) in portions at 0° C. under nitrogen atmosphere. The reaction was monitored by LCMS. The reaction was quenched by the addition of water/ice (800 mL) at 0° C. The aqueous layer was extracted with ethyl acetate (EtOAc) (3×800 mL). The resulting mixture was concentrated under reduced pressure. The residue was purified by silica gel column chromatography, eluting with petroleum ether/ethyl acetate (3:2) to afford {2-[(tert-butyldiphenylsily 1)oxy]-3-(hydroxymethyl)cyclopentyl}methanol (13 g. 64% yield). LCMS: (ES, m/z): RT=1.25 min, m/z=307.2 [M+H]⁺.

Step 5. Into a 40 mL vial was added {2-[(tert-butyldiphenylsilyl)oxy]-3-(hydroxymethyl)cyclopentyl}methanol (100 g. 2.60 mmol, 1 equiv), toluene (10 mL), tetramethylazodicarboxamide (TMAD) (1.34 g. 7.80 mmol, 3 equiv), and tri-n-butylphosphine (n-Bu3P) (1.58 g. 7.80 mmol, 3 equiv) at room temperature. The resulting mixture was stirred for 2b at 80° C. under nitrogen atmosphere. The reaction was monitored by GCMS. The reaction was quenched by the addition of water (200 mL) at room temperature. The aqueous layer was extracted with ethyl acetate (EtOAc) (3×200 mL). The resulting mixture was concentrated under reduced pressure. The residue was purified by reversed-phase flash chromatography Method Z to provide tert-butyl({3-oxabicyclo[3.2.1]octan-8-yloxy})diphenylsilane (600 mg, 63% yield). GCMS: (ES, m/z): RT=7.5 min, m/z=366.2 [M]⁺.

Step 6. Into a 40 mL vial was added tert-butyl({3-oxabicyclo[3.2.1]octan-8-yloxy})diphenylsilane (2 g, 5.45 mmol, 1 equiv) and tetrahydrofuran (THF) (3 mL), tetra-butylammonium fluoride (TBAF) (10.9 mL. 10.9 mmol, 2 equiv) (IM in THF) at room temperature. The resulting mixture was stirred for overnight at room temperature. The reaction was monitored by TLC. The resulting mixture was extracted with ethyl acetate (EtOAc) (3×50 mL). The combined organic layers were washed with water (3×10 mL), dried over anhydrous Na₂SO₄. After filtration, the filtrate was concentrated under reduced pressure. The residue was purified by silica gel column chromatography, eluting with petroleum ether/ethyl acetate (5:3) to afford 3-oxabicyclo[3.2.1]octan-8-ol (600 mg, 86% yield). GCMS: (ES, m/z): RT=4.5 min, m/z=128.1 [M]⁺.

Step 7. To a stirred solution of (COCl)₂ (5.85 mL, 11.7 mmol. 1.5 equiv) in dichloromethane (DCM) (10 mL) was added dimethylsulfoxide (DMSO) (1.22 g. 15.6 mmol, 2 equiv) in DCM (20 mL) dropwise at −78° C. under nitrogen atmosphere. The resulting mixture was stirred for 15 min at −78° C. under nitrogen atmosphere. To the above mixture was added 3-oxabicyclo[3.2.1]octan-8-ol mixture from step 7 (1 g. 7.80 mmol, 1 equiv) in 5 mL DCM by dropwise over 5 min at −78° C. To the above mixture was added triethylamine (TEA) (3.95 g, 39.0 mmol, 5 equiv) in 5 mL DCM dropwise over 5 min at −78° C. The resulting mixture was stirred for additional 20 min at room temperature. The reaction was quenched by the addition of water (20 mL) at room temperature. The mixture adjusted to pH 6 with HCl (aq.). The resulting mixture was extracted with DCM (3×30 mL). The combined organic layers were washed with water (3×10 mL.), dried over anhydrous Na₂SO₄. After filtration, the filtrate was concentrated under reduced pressure. The residue was purified by silica gel column chromatography, eluting with DCM/petroleum ether (12:1), to afford 3-oxabicyclo[3.2.1]octan-8-one (700 mg, 71% yield). GC MS: (ES, m/z): RT=3.20 min, m/z=126.1 [M]⁺.

Step 8. A solution of 3-oxabicyclo[3.2.1]octan-8-one (1 g. 7.92 mmol, 1 equiv) and benzylamine (NH₂Bn) (1.27 g, 11.9 mmol, 1.5 equiv), titanium isopropoxide (Ti(OiPr)₄) (2.25 g. 7.92 mmol, 1 equiv) in ethanol (EtOH) (50 mL) was stirred for overnight at 60° C. under nitrogen atmosphere. To the above mixture was added NaBH₄ (0.45 g, 11.9 mmol, 1.5 equiv) in portions over min at room temperature. The resulting mixture was stirred for additional 2 h at 60° C. The reaction was monitored by LCMS. The reaction was quenched by the addition of water (100 mL.) at room temperature. The aqueous layer was extracted with ethyl acetate (EtOAc) (3×100 mL) and dried over anhydrous Na₂SO₄. After filtration, the filtrate was concentrated under reduced pressure. The residue was purified by reversed-phase flash chromatography Method AA to provide N-benzyl-(1R,5S,8s)-3-oxabicyclo[3.2.1]octan-8-amine (1 g, 58% yield). LCMS: (ES, m/z): RT=0.51 min, m/z=218.2 [M+H]⁺. Stereochemistry retroactively assigned based on absolute stereochemical determination of Compound 6A and comparison of this product to the corresponding deprotected (1R,5S,8s)-3-oxabicyclo[3.2.1]octan-8-amine prepared from Example 4, step 1.

Step 9. A solution of N-benzyl-(1R,5S,8s)-3-oxabicyclo[3.2.1]octan-8-amine (1 g, 4.60 mmol, 1 equiv) and Pd/C (0.98 g) (10 wt % on carbon) in methanol (MeOH) was stirred for overnight at room temperature under hydrogen atmosphere. The reaction was monitored by LCMS. The resulting mixture was filtered, the filter cake was washed with MeOH (3×10 mL) were added HCl (gas) in 1,4-dioxane (1.73 mL, 6.90 mmol, 1.5 equiv) at room temperature. The filtrate was concentrated under reduced pressure to provide (1R,5S,8s)-3-oxabicyclo[3.2.1]octan-8-amine (600 mg. 80% yield). LCMS: (ES, m/z): RT=1.36 min, m/z=451.1 [M+H]⁺.

Step 10. Into a 40 mL vial was added 3-oxabicyclo[3.2.1]octan-8-amine hydrochloride (600 mg. 3.66 mmol, 1 equiv) and 1,4-dichloropyrido[3,4-d]pyridazine (807 mg, 4.03 mmol, 1.10 equiv), triethylamine (TEA) (1.11 g. 11.0 mmol, 3 equiv), dimethylsulfoxide (DMSO) (10 mL) at room temperature. The resulting mixture was stirred for 2 h at 100° C. under nitrogen atmosphere. The reaction was monitored by LCMS. The reaction was quenched by the addition of water (100 mL) at room temperature. The resulting mixture was extracted with ethyl acetate (EtOAc) (3×100 mL). The combined organic layers were washed with water (2×100 mL.), dried over anhydrous Na—SO₄. After filtration, the filtrate was concentrated under reduced pressure. The residue was purified by reversed-phase flash chromatography Method BB to provide a mixture of major and minor 1R,5S,8s regioisomers. N-((1R,5S,8S)-3-oxabicyclo[3.2.1]octan-8-1)-1-chloropyrido[3,4-d]pyridazin-4-amine (major isomer) and N-((1R,5S,8S)-3-oxabicyclo[3.2.1]octan-8-yl)-4-chloropyrido[3,4-d]pyridazin-1-amine (minor isomer) (850 mg. 79.7% yield). LCMS: (ES, m/z): RT=1.36 min, m/z=451.1 [M+H]⁺.

Step 11. Into a 8 mL vial was added 3-fluoro-2-hydroxy-4-methylphenylboronic acid (204.58 mg, 1.21 mmol, 2.5 equiv) and dioxane (2 mL). 1-chloro-N-(3-oxabicyclo[3.2.1]octan-8-ylpyrido[3,4-d]pyridazin-4-amine (mixture, 140 mg. 0.48 mmol, 1 equiv), Na₂CO₃ (155 mg, 1.45 mmol, 3 equiv). [1,1′-bis(diphenylphosphino) ferrocene]dichloro palladium (II) dichloromethane complex ((Pd(dppf)Cl₂CH₂Cl₂) (106 mg, 0.15 mmol, 0.3 equiv), and water (0.4 mL). The resulting mixture was stirred for 2 h at 80° C. under nitrogen atmosphere. The reaction was monitored by LCMS. The resulting mixture was filtered, the filter cake was washed with methanol (MeOH) (3×30 mL). The filtrate was concentrated under reduced pressure. The residue was purified by reversed-phase flash chromatography Method CC to the crude product mixture (100 mg. 80% purity), which was further purified by Prep-HPLC Method DD afford 6-(1-(((1R,5S,8s)-3-oxabicyclo[3.2.1]octan-8-yl)amino)pyrido[3,4-d]pyridazin-4-yl)-2-fluoro-3-methylphenol (Compound 15A-regioisomer) as the minor isomer (2.3 mg, 1.13% yield: RT (min): 12.0) and 6-(4-(((1R,5S,8s)-3-oxabicyclo[3.2.1]octan-8-yl)amino)pyrido[3,4-d]pyridazin-1-yl)-2-fluoro-3-methylphenol (Compound 15A) as the major isomer (22.4 mg, 12.2% yield: RT (min): 13.5). Stereochemistry of Compound 15A and Compound 15A-regioisomer retroactively assigned based on absolute stereochemical determination of Compound 6A and comparison of the N-Bn protected amine used in this Example to the corresponding deprotected (1R,5S,8s)-3-oxabicyclo[3.2.1]octan-8-amine prepared from Example 4, step 1.

Compound 15A: LCMS: (ES, m/z): RT=1.06 min, m/z=381.2 [M+H]⁺. ¹H NMR (400 MHZ, DMSO-d₆) δ 9.95 (s, 1H), 9.80 (d, J=31.6 Hz, 1H), 8.88 (d, J=5.6 Hz, 1H), 7.80 (d, J=3.6 Hz, 1H). 7.36 (d, J=5.6 Hz, 1H). 7.05 (d, J=7.8 Hz, 1H), 6.85 (s, 1H), 4.25-3.95 (m, 3H), 3.45-3.34 (m, 2H), 2.48 (s, 2H), 2.32 (d, J=2.4 Hz, 3H), 1.97-1.75 (m, 4H).

Compound 15A-regioisomer: LCMS: (ES, m/z): RT=1.19 min, m/z=381.2 [M+H]⁺. ¹H NMR (400 MHZ, DMSO-d₆) δ 9.57 (s, 1H), 8.99 (d, J=5.4 Hz, 1H), 8.89 (d, J=0.8 Hz, 1H), 8.50 (dd, J=5.8, 1.0 Hz, 1H), 7.62 (d, J=3.6 Hz, 1H), 7.12 (dd, J=7.6 Hz, 1H), 6.89 (t, J=7.6 Hz, 1H), 4.11-4.01 (m, 3H), 3.43-3.34 (m, 2H), 2.47 (s, 2H), 2.34 (d, J=2.4 Hz, 3H), 1.93-1.74 (m, 4H).

### Example 9. 2-(4-(((1R,5S,8s)-3-oxabicyclo[3.2.1]octan-8-yl)amino)pyrido[3,4-d]pyridazin-1-yl)-3-fluoro-5-methylphenol (Compound 16A) and 2-(1-(((1R,5S,8s)-3-oxabicyclo[3.2.1]octan-8-yl)amino)pyrido[3,4-d]pyridazin-4-yl)-3-fluoro-5-methylphenol (Compound 16A-regioisomer)

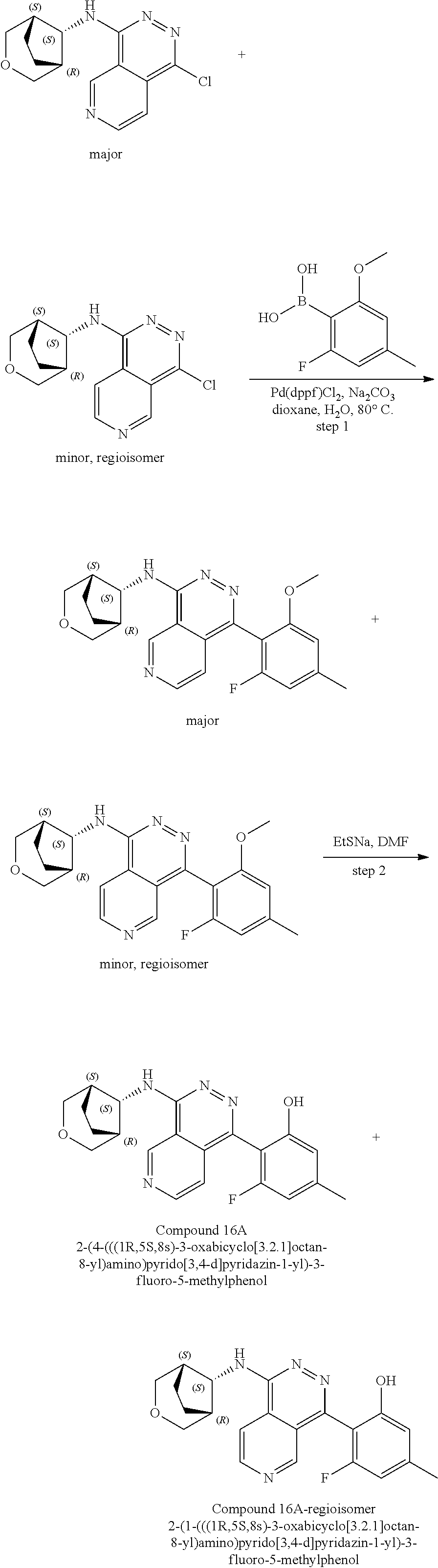

Step 1. Into a 40 mL vial were added 2-fluoro-6-methoxy-4-methylphenylboronic acid (200.0 mg, 1.08 mmol, 1 equiv) and a mixture of N-((1R,5S,8S)-3-oxabicyclo[3.2.1]octan-8-yl)-1-chloropyrido[3,4-d]pyridazin-4-amine (major isomer) and N-((1R,5S,8S)-3-oxabicyclo[3.2.1]octan-8-yl)-4-chloropyrido[3,4-d]pyridazin-1-amine (minor isomer) from Example 8, step 10 (316.1 mg, 1.08 mmol, 1 equiv), [1,1′-bis(diphenylphosphino) ferrocene]dichloro palladium (II) dichloromethane complex ((Pd(dppf)Cl₂CH₂Cl₂) (159.1 mg, 0.21 mmol, 0.20 equiv), Na₂CO₃ (345.7 mg. 3.26 mmol, 3 equiv), dioxane (10 mL), H₂O (2 mL) at room temperature. The resulting mixture was stirred for 2 h at 80° C. under nitrogen atmosphere. The reaction was monitored by LCMS. The reaction was quenched by the addition of water (200 mL) at room temperature. The aqueous layer was extracted with ethyl acetate (EtOAc) (3×100 mL) The resulting mixture was concentrated under reduced pressure. The residue was purified by silica gel column chromatography, eluting with petroleum ether/ethyl acetate (1:20) to afford a mixture of amine regioisomers: N-((1R,5S,8s)-3-oxabicyclo[3.2.1]octan-8-yl)-1-(2-fluoro-6-methoxy-4-methylphenyl)pyrido[3,4-d]pyridazin-4-amine (major isomer) and N-((1R,5S,8s)-3-oxabicyclo[3.2.1]octan-8-yl)-4-(2-fluoro-6-methoxy-4-methylphenyl)pyrido[3,4-d]pyridazin-1-amine (minor isomer) (190 mg, 44.3% yield). LCMS: (ES, m/z): RT=0.55 min, m/z=395.1 [M+H]⁺.

Step 2. Into a 40 mL vial were added the amine mixture of step 1 (170 mg. 0.43 mmol, 1 equiv) and (ethylsulfanyl) sodium (EtSNa) (543.8 mg, 6.46 mmol. 15.0 equiv), dimethylformamide (DMF) (10 mL) at room temperature. The resulting mixture was stirred for 2 h at 120° C. under nitrogen atmosphere. The reaction was monitored by LCMS. The reaction was quenched by the addition of water (0.5 mL) at room temperature. The residue was extracted with dichloromethane (DCM) (3×20 ml), and the organic phase was dried over Na—SO₄, filtered, and the filtrate concentrated under reduce pressure. The residue was purified by reversed-phase flash chromatographic Method GG to afford the crude product (150 mg. 86% purity), which was purified by Prep-HPLC Method HH to afford 2-(1-(((1R,5S,8s)-3-oxabicyclo[3.2.1]octan-8-yl)amino)pyrido[3,4-d]pyridazin-4-yl)-3-fluoro-5-methylphenol (Compound 16A-regioisomer) (8.3 mg, 5.1% yield. RT (min): 16.2) as the minor isomer, and 2-(4-(((1R,5S,8s)-3-oxabicyclo[3.2.1]octan-8-yl)amino)pyrido[3,4-d]pyridazin-1-yl)-3-fluoro-5-methylphenol (Compound 16A) (35.4 mg. 21.6% yield, RT (min): 17.3) as the major isomer. Stereochemistry of Compound 16A and Compound 16A-regioisomer retroactively assigned based on the rationale as set forth in Example 10.

Compound 16A: LCMS: (ES, m/z): RT=1.22 min, m/z=381.1 [M+H]+. 1H NMR (400 MHZ, DMSO-d₆) δ 9.97 (d, J=1.0 Hz, 1H), 9.85 (s, 1H), 8.88 (d, J=5.6 Hz, 1H), 7.83 (d, J=3.6 Hz, 1H), 7.22 (d, J=5.6 Hz, 1H), 6.66 (d, J=10 Hz, 2H), 4.14-4.09 (m, 3H), 3.38-3.32 (m, 2H), 2.49-2.46 (m, 2H), 2.34 (s, 3H), 1.89-1.81 (m, 4H).

Compound 16A-regioisomer: LCMS: (ES, m/z): RT=0.55 min, m/z=381.1[M+H]. ¹H NMR (400 MHz, DMSO-d₆) δ 9.98 (s, 1H), 9.00 (d, J=5.6 Hz, 1H), 8.77 (s, 1H), 8.52-8.51 (d, J=5.6 Hz. 1H), 7.63 (d, J=3.2 Hz, 1H), 6.68 (d, J=8.4 Hz, 2H), 4.08-4.05 (m, 3H), 3.38-3.32 (m, 2H), 2.46-2.45 (m, 2H), 2.35 (s, 3H), 1.99-1.72 (m, 4H).

### Example 10. 2-(4-(((1R,5S,8s)-3-oxabicyclo[3.2.1]octan-8-yl)amino)pyrido[3,4-d]pyridazin-1-yl)-3,5-dimethylphenol (Compound 17A) and 2-(1-(((1R,5S,8s)-3-oxabicyclo[3.2.1]octan-8-yl)amino)pyrido[3,4-d]pyridazin-4-yl)-3,5-dimethylphenol (Compound 17A-regioisomer)

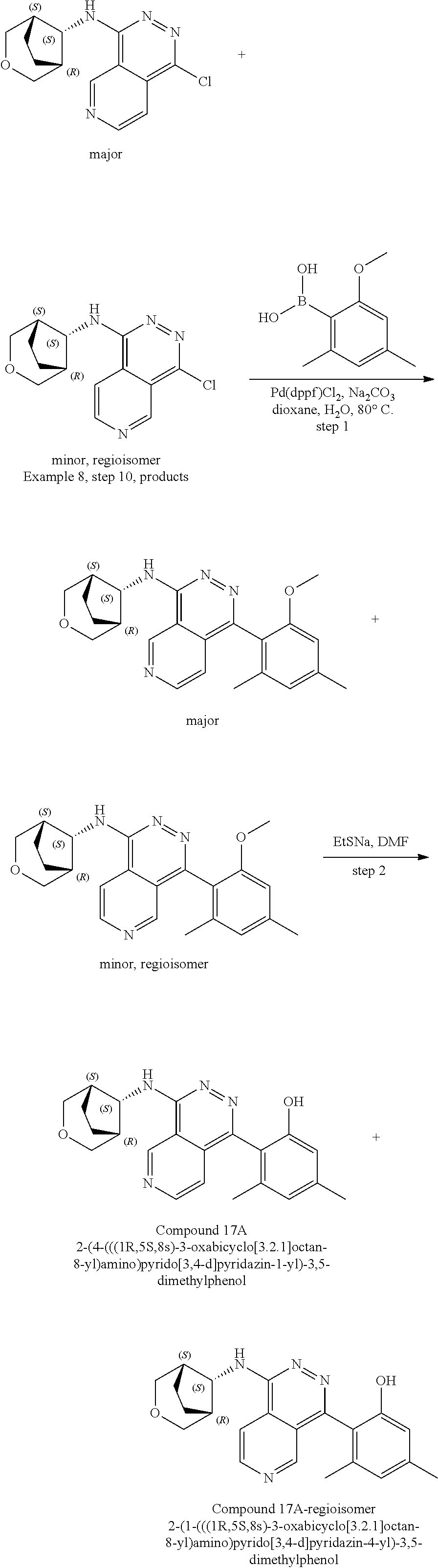

Step 1. Into a 40 mL vial were added 2-methoxy-4,6-dimethylphenylboronic acid (200.0 mg, 1.11 mmol, 1 equiv) and a mixture of N-((1R,5S,8S)-3-oxabicyclo[3.2.1]octan-8-yl)-1-chloropyrido[3,4-d]pyridazin-4-amine (major isomer) and N-((1R,5S,8S)-3-oxabicyclo[3.2.1]octan-8-yl)-4-chloropyrido[3,4-d]pyridazin-1-amine (minor isomer) from Example 8, step 10 (323.0 mg, 1.11 mmol, 1 equiv), [1,1′-bis(diphenylphosphino) ferrocene]dichloro palladium (II) dichloromethane complex ((Pd(dppf)Cl₂CH₂Cl₂) (162.6 mg, 0.22 mmol, 0.20 equiv), Na₂CO₂ (353.3 mg, 3.33 mmol, 3 equiv), dioxane (10 mL), H₂O (2 mL) at room temperature. The resulting mixture was stirred for 2 h at 80° C. under nitrogen atmosphere. The reaction was monitored by LCMS. The reaction was quenched by the addition of water (200 mL.) at room temperature. The aqueous layer was extracted with ethyl acetate (EtOAc) (3×100 mL) and the organic phase was concentrated under reduced pressure. The residue was purified by silica gel column chromatography, eluting with petroleum ether:ethyl acetate (1:25), to afford a mixture of N-((1R,5S,8s)-3-oxabicyclo[3.2.1]octan-8-yl)-1-(2-methoxy-4,6-dimethylphenyl)pyrido[3,4-d]pyridazin-4-amine (major isomer) and N-((1R,5S,8s)-3-oxabicyclo[3.2.1]octan-8-yl)-4-(2-methoxy-4,6-dimethylphenyl)pyrido[3,4-d]pyridazin-1-amine (minor isomer) (150 mg. 35% yield). LCMS: (ES, m/z): RT-0.55 min, m/z=391.1 [M+H].

Step 2. Into a 40 mL vial was added the amine mixture of step 1 (130 mg, 0.33 mmol, 1 equiv) and (ethylsulfanyl) sodium (EtSNa) (420.1 mg. 4.95 mmol, 15.0 equiv), and dimethylformamide (DMF) (8 mL) at room temperature. The resulting mixture was stirred for 2 h at 120° C. under nitrogen atmosphere. The reaction was monitored by LCMS. The reaction was quenched by the addition of water (5 mL) at room temperature. The residue was extracted with dichloromethane (DCM) (3×15 ml), and the organic phase was concentrated under vacuum. The residue was purified by reversed-phase flash chromatographic Method GG to afford the crude product (100 mg. 85% purity). The crude product was then purified by Prep-HPLC Method HH to afford 2-(1-(((1R,5S,8s)-3-oxabicyclo[3.2.1]octan-8-yl)amino)pyrido[3,4-d]pyridazin-4-yl)-3,5-dimethylphenol (Compound 17A-regioisomer) (2.5 mg, 2.0% yield, RT (min): 16.2) as the minor isomer, and 2-(4-(((1R,5S,8s)-3-oxabicyclo[3.2.1]octan-8-yl)amino)pyrido[3,4-d]pyridazin-1-yl)-3,5-dimethylphenol (Compound 17A) (20.8 mg, 16.6% yield, RT (min): 19) as the major isomer. Stereochemistry of Compound 17A and Compound 17A-regioisomer retroactively assigned based on the rationale as set forth in Example 10.

Compound 17A: LCMS: (ES, m/z): RT=0.57 min, m/z=377.1 [M+H]⁺. 1H NMR (400 MHZ, DMSO-d₆) δ 9.95 (s, 1H), 9.23 (s, 1H), 8.84 (d, J=5.6 Hz, 1H), 7.71 (d, J=3.6 Hz, 1H), 7.12 (d, J=5.6, 1H), 6.65 (d, J=5.6 Hz, 2H), 4.20-4.11 (m, 2H), 4.08 (d, J=11.2 Hz, 1H). 3.40-3.34 (m, 2H), 2.41 (s. 1H), 2.29 (s, 3H). 1.88 (s, 3H), 1.89-1.87 (m, 4H).

Compound 17A-regioisomer: LCMS: (ES, m/z): RT=0.56 min, m/z=377.1 [M+H]⁺. 1H NMR (400 MHZ, DMSO-d₆) δ 9.29 (s, 1H), 8.97 (d, J=5.6 Hz, 1H), 8.66 (s, 1H), 8.50 (d, J=5.6 Hz, 1H), 7.51 (d, J=3.6 Hz, 1H), 6.67 (d, J=8.4 Hz, 2H), 4.14-4.10 (m, 2H), 4.03 (d, J=10.8 Hz, 1H), 3.37-3.36 (m, 2H), 2.40 (s, 2H), 2.30 (s, 3H), 1.92 (s, 3H). 1.92-1.79 (m, 4H).

### Example 11. 4-(4-(((1R,5S,8s)-3-oxabicyclo[3.2.1]octan-8-yl)amino)pyrido[3,4-d]pyridazin-1-yl)-3-hydroxybenzonitrile (Compound 18A) and 4-(1-(((1R,5S,8s)-3-oxabicyclo[3.2.1]octan-8-yl)amino)pyrido[3,4-d]pyridazin-4-yl)-3-hydroxybenzonitrile (Compound 18A-regioisomer)

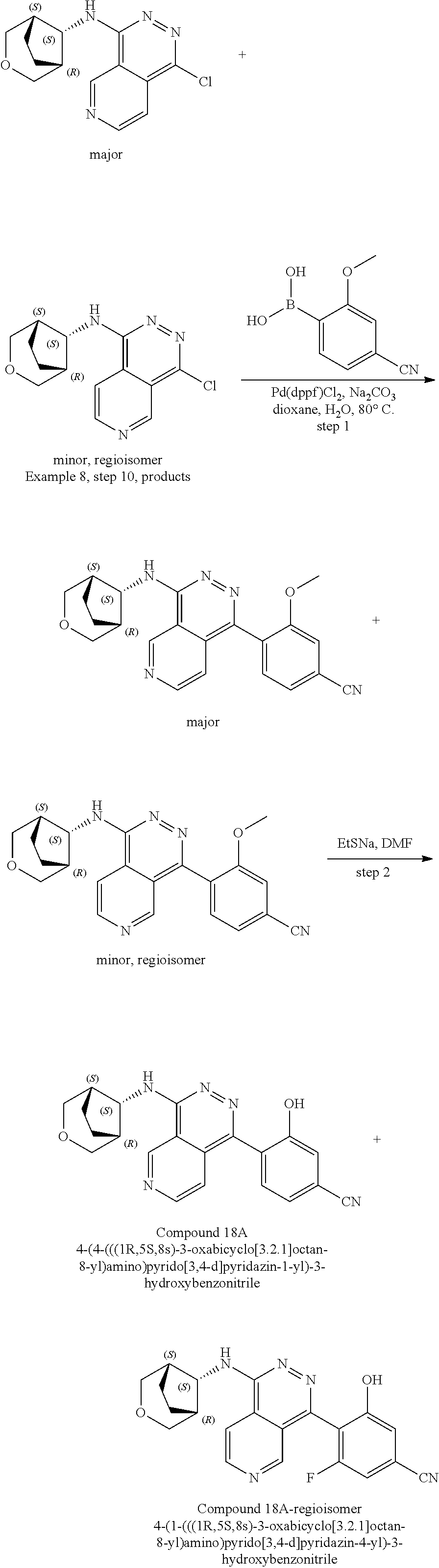

Step 1. Into a 40 mL vial were added 4-cyano-2-methoxyphenylboronic acid (200.0 mg, 1.13 mmol, 1 equiv) and a mixture of N-((1R,5S,8S)-3-oxabicyclo[3.2.1]octan-8-yl)-1-chloropyrido[3,4-d]pyridazin-4-amine (major isomer) and N-((1R,5S,8S)-3-oxabicyclo[3.2.1]octan-8-yl)-4-chloropyrido[3,4-d]pyridazin-1-amine (minor isomer) from Example 8, step 10 (328.7 mg, 1.13 mmol, 1 equiv), [1,1′-bis(diphenylphosphino) ferrocene]dichloro palladium (II) dichloromethane complex ((Pd(dppf)Cl₂ CH₂Cl₂) (165.4 mg, 0.22 mmol, 0.20 equiv), Na₂CO₃ (359.3 mg, 3.39 mmol, 3 equiv), dioxane (10 mL), H₂O (2 mL) at room temperature. The resulting mixture was stirred for 2 h at 80° C. under nitrogen atmosphere. The reaction was monitored by LCMS. The reaction was diluted by the addition of water (200 mL) at room temperature. The aqueous layer was extracted with ethyl acetate (EtOAc) (3×100 mL), the organic phase was concentrated under reduced pressure, and the residue was purified by silica gel column chromatography, eluting with petroleum ether:ethyl acetate (1:30) to afford a mixture of 4-(4-(((1R,5S,8s)-3-oxabicyclo[3.2.1]octan-8-yl)amino)pyrido[3,4-d]pyridazin-1-yl)-3-methoxybenzonitrile (major isomer) and 4-(1-(((1R,5S,8s)-3-oxabicyclo[3.2.1]octan-8-yl)amino)pyrido[3,4-d]pyridazin-4-yl)-3-methoxybenzonitrile (minor isomer) (230 mg, 53% yield). LCMS: (ES, m/z): RT=0.49 min, m/z=388.1 [M+H]⁺.

Step 2. Into a 40 mL vial were added a mixture of amines from step 1 (210 mg, 0.54 mmol, 1 equiv), (ethylsulfanyl) sodium (EtSNa) (683.9 mg. 8.13 mmol. 15.0 equiv), and dimethylformamide (DMF) (10 mL) at room temperature. The resulting mixture was stirred for 2 h at 120° C. under nitrogen atmosphere. The reaction was monitored by LCMS. The reaction was quenched by the addition of water (5 mL) at room temperature. The residue was extracted with dichloromethane (DCM) (3×15 ml), and the organic phase was concentrated under vacuum. The residue was purified by reversed-phase flash chromatographic Method GG to afford the crude product (160 mg. 90% purity). Then the crude product was purified by Prep-HPLC Method II to afford 4-(1-(((1R,5S,8s)-3-oxabicyclo[3.2.1]octan-8-1)amino)pyrido[3,4-d]pyridazin-4-yl)-3-hydroxy benzonitrile (Compound 18A-regioisomer) (12.1 mg, 6% yield. RT (min): 17.3) as the minor isomer and 4-(4-(((1R,5S,8s)-3-oxabicyclo[3.2.1]octan-8-yl)amino)pyrido[3,4-d]pyridazin-1-yl)-3-hydroxybenzonitrile (Compound 18A) (46.5 mg. 23% yield, RT (min): 20.8) as the major isomer. Stereochemistry of Compound 18A and Compound 18A-regioisomer retroactively assigned based on the rationale as set forth in Example 10.

Compound 18A: LCMS: (ES, m/z): RT=1.20 min, m/z=374.1 [M+H]+. 1H NMR (400 MHZ, DMSO-d₆) δ 10.59 (s, 1H), 9.97 (s, 1H), 8.89 (d, J=5.6 Hz, 1H), 7.88 (d, J=3.6 Hz. 1H), 7.55 (d, J=8.0 Hz, 1H), 7.43 (dd, J=10.8, 1.2 Hz, 1H), 7.34 (d, J=1.6 Hz, 1H), 7.30 (d, J=5.6 Hz, 1H), 4.17-4.06 (m, 3H), 3.51-3.36 (m, 2H). 2.48 (s, 2H). 1.89-1.79 (m, 4H).

Compound 18A-regioisomer: LCMS: (ES, m/z): RT=1.21 min, m/z=374.1[M+H]⁺. 1H NMR (400 MHz, DMSO-d₆) δ 10.73 (s, 1H), 8.99 (d, J=5.6 Hz. JH). 8.86 (s, 1H), 8.51 (d, J=5.6 Hz, 1H), 7.67 (d, J=2.8 Hz, 1H), 7.59 (d, J=8.0 Hz, 1H), 7.44 (d, J=7.6, 1H), 7.33 (d, J=1.6 Hz, 1H), 4.13-4.03 (m. 3H). 3.55-3.34 (m, 2H), 2.33-2.31 (m, 2H), 1.89-1.80 (m, 4H).

### Example 12. 2-(4-((7-oxabicyclo[2.2.1]heptan-2-yl)amino)pyrido[3,4-d]pyridazin-1-yl)-5-(trifluoromethyl) phenol (Compound 10*), 2-(4-(((2R)-7-oxabicyclo[2.2.1]heptan-2-yl)amino)pyrido[3,4-d]pyridazin-1-yl)-5-(trifluoromethyl) phenol (Compound 10′***), 2-(4-(((2S)-7-oxabicyclo[2.2.1]heptan-2-yl)amino)pyrido[3,4-d]pyridazin-1-yl)-5-(trifluoromethyl) phenol (Compound 10″″*), 2-(4-(((1R,2R,4S)-7-oxabicyclo[2.2.1]heptan-2-yl)amino)pyrido[3,4-d]pyridazin-1-yl)-5-(trifluoromethyl) phenol (Compound 10A*), 2-(4-(((1R,2S,4S)-7-oxabicyclo[2.2.1]heptan-2-yl)amino)pyrido[3,4-d]pyridazin-1-yl)-5-(trifluoromethyl) phenol (Compound 10B*), 2-(4-(((1S,2R,4R)-7-oxabicyclo[2.2.1]heptan-2-yl)amino)pyrido[3,4-d]pyridazin-1-yl)-5-(trifluoromethyl) phenol (Compound 10C*), and 2-(4-(((1S,2S,4R)-7-oxabicyclo[2.2.1]heptan-2-yl)amino)pyrido[3,4-d]pyridazin-1-yl)-5-(trifluoromethyl) phenol (Compound 10D*)

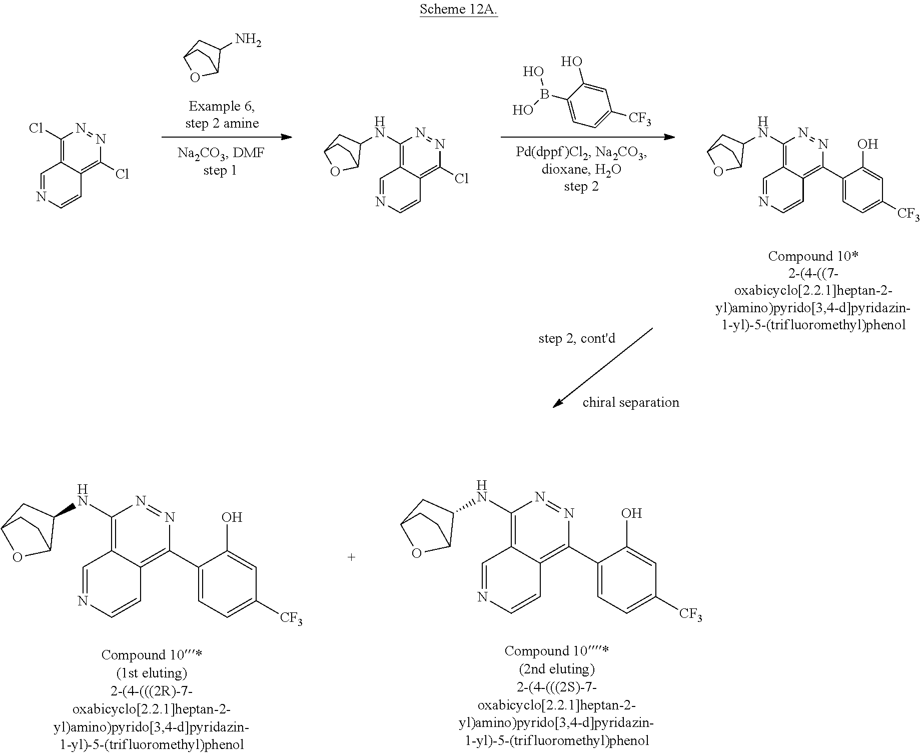

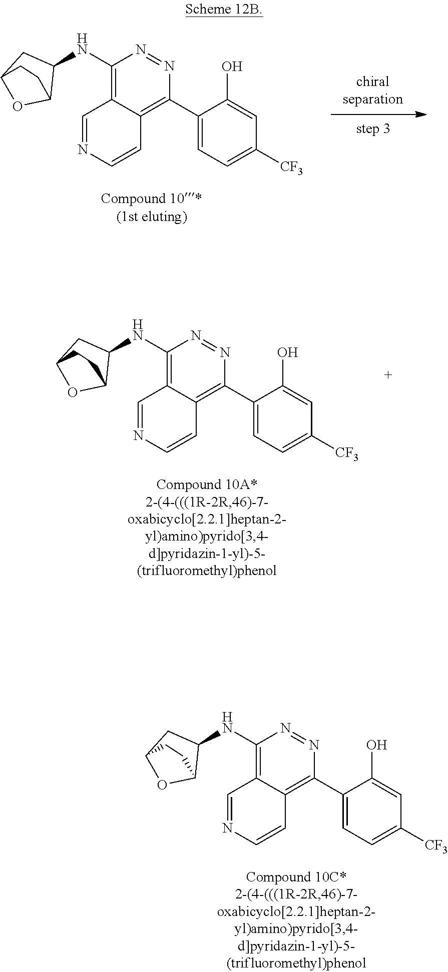 [SMILES: Oc1cc(C(F)(F)F)ccc1-c1nnc(N[C@@H]2CC3CCC2O3)c2cnccc12 ; Oc1cc(C(F)(F)F)ccc1-c1nnc(N[C@@H]2C[C@@H]3CC[C@H]2O3)c2cnccc12 ; Oc1cc(C(F)(F)F)ccc1-c1nnc(N[C@@H]2C[C@H]3CC[C@@H]2O3)c2cnccc12]

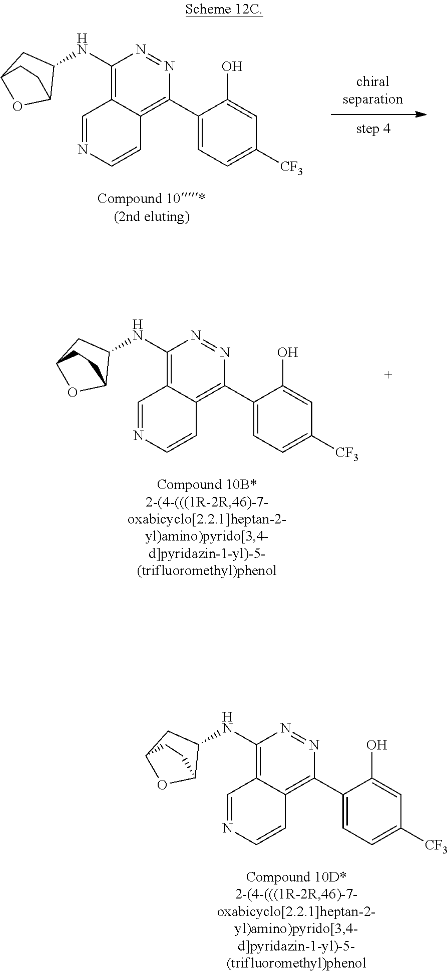 [SMILES: Oc1cc(C(F)(F)F)ccc1-c1nnc(N[C@H]2CC3CCC2O3)c2cnccc12 ; Oc1cc(C(F)(F)F)ccc1-c1nnc(N[C@H]2C[C@@H]3CC[C@H]2O3)c2cnccc12 ; Oc1cc(C(F)(F)F)ccc1-c1nnc(N[C@H]2C[C@H]3CC[C@@H]2O3)c2cnccc12]

Step 1. Into a 20 mL vial was added 1,4-dichloropyrido[3,4-d]pyridazine (600 mg, 3 mmol, 1 equiv), 7-oxabicyclo[2.2.1]heptan-2-amine (from Example 6, step 2) hydrochloride salt (894 mg, 6 mmol, 2 equiv), Na₂CO₃ (954 mg. 9 mmol, 3 equiv) and dimethylformamide (DMF) (5 mL). The reaction mixture was irradiated with microwave radiation for 0.5 h at 130° C., and then the reaction was quenched with water (10 mL). The resulting mixture was extracted with ethyl acetate (EtOAc) (3×10 mL), dried over anhydrous Na₂SO₄, filtered, and then the filtrate was concentrated under reduced pressure. The residue was purified by reversed-phase flash chromatography Method JJ to provide a crude product (700 mg) which was purified by Prep-HPLC Method KK to afford 1-chloro-N-{7-oxabicyclo[2.2.1]heptan-2-yl}pyrido[3,4-d]pyridazin-4-amine (140 mg, 72% yield). LC MS: (ES, m/z): RT=0.76 min, m/z=277.0 [M+H]⁺.

Step 2. Into a 8 mL vial was added 1-chloro-N-{7-oxabicyclo[2.2.1]heptan-2-yl}pyrido[3,4-dipyridazin-4-amine (140 mg, 0.51 mmol, 1 equiv). 2-hydroxy-4-(trifluoromethyl)phenylboronic acid (125 mg. 0.61 mmol. 1.3 equiv), Na₂CO₃ (162.40 mg, 1.53 mmol. 3 equiv). 1,1′-bis(diphenylphosphino) ferrocene]dichloro palladium (II) dichloromethane complex ((Pd(dppf)Cl₂CH₂Cl₂) (111.1 mg, 0.15 mmol, 0.30 equiv), dioxane (2 mL) and H₂O (0.4 mL) at 80° C. The resulting mixture was stirred for 2 h at 80° C. under nitrogen atmosphere. The reaction was monitored by LCMS. The resulting mixture was diluted with water (10 mL). The resulting mixture was extracted with ethyl acetate (EtOAc) (3×50 mL). The combined organic layers were washed with water (2×40 mL), dried over anhydrous Na₂SO₄. After filtration, the filtrate was concentrated under reduced pressure. The residue was purified by reversed-phase flash chromatography Method LL to provide 2-(4-((7-oxabicyclo[2.2.1]heptan-2-yl)amino)pyrido[3,4-d]pyridazin-1-yl)-5-(trifluoromethyl) phenol (Compound 10*) (120 mg), which was further purified by Prep-HPLC Method MM to afford 2-(4-(((2R)-7-oxabicyclo[2.2.1]heptan-2-ylamino)pyrido[3,4-d]pyridazin-1-yl)-5-(trifluoromethyl) phenol (Compound 10′″*) (65 mg) as the first eluting peak, and 2-(4-(((2S)-7-oxabicyclo[2.2.1]heptan-2-yl)amino)pyrido[3,4-d]pyridazin-1-yl)-5-(trifluoromethyl) phenol (Compound 10″″*) (10 mg) as the second eluting peak, each comprising a mixture of two stereoisomers. LCMS: (ES, m/z): RT=1.28 min, m/z=403.0 [M+H]+. Stereochemistry was arbitrarily assigned.

Step 3. The Is eluting mixture of step 2 (Compound 10″*) (65 mg) was purified by Prep-Chiral-HPLC Method NN to afford 2-(4-(((1S,2R,4R)-7-oxabicyclo[2.2.1]heptan-2-yl)amino)pyrido[3,4-d]pyridazin-1-yl)-5-(trifluoromethyl)phenol (Compound 10C*) (23.4 mg, 12% yield: RT (min): 10.77) as the first eluting peak, and 2-(4-(((1R,2R,4S)-7-oxabicyclo[2.2.1]heptan-2-yl)amino)pyrido[3,4-d]pyridazin-1-yl)-5-(trifluoromethyl) phenol (Compound 10A*) (22.7 mg, 11% yield: RT (min): 12.14) as the second eluting peak. Stereochemistry was arbitrarily assigned.

Compound 10A*: LCMS: (ES, m/z): RT=0.67 min, m/z=403.1 [M+H]⁺: ¹H NMR (400 MHZ, Methanol-d₄) δ 9.96 (d, J=0.9 Hz, 1H), 9.07 (d, J=5.6 Hz, 1H), 7.65-7.58 (m, 2H), 7.38 (m, J=8.0, 1.4 Hz, 1H), 7.31 (d, J=1.7 Hz, 1H), 4.84-4.78 (m, 2H), 4.31 (m. J=7.8, 3.1 Hz, 1H), 2.29 (m, J=13.0, 7.8 Hz, 1H), 2.11-1.99 (m, 1H), 1.94-1.70 (m, 3H), 1.69-1.58 (m, 1H).

Compound 10C*: LCMS: (ES, m/z): RT=1.14 min, m/z=403.1[M+H]⁺; ¹H NMR (400 MHz, Methanol-d₄) δ 9.89 (s, 1H), 9.00 (d, J=5.6 Hz, 1H), 7.65-7.54 (m, 2H), 7.37 (d, J=7.8 Hz, 1H), 7.31 (s, 1H), 4.77 (d, J=5.7 Hz, 2H), 4.36 (m, J=7.9, 3.2 Hz, 1H), 2.27 (m, J=12.9, 7.9 Hz, 1H), 2.05 (s, 1H), 1.88-1.68 (m, 3H), 1.63 (m, J=11.3, 7.2 Hz, 1H).

Step 4. The 2″d eluting mixture of step 2 (Compound 10″″*) (10 mg) was purified by Prep-Chiral-HPLC Method 00 to afford 2-(4-(((1S,2S,4R)-7-oxabicyclo[2.2.1]heptan-2-yl)amino)pyrido[3,4-d]pyridazin-1-yl)-5-(trifluoromethyl) phenol (Compound 10D*) (3.9 mg. 3% yield; RT (min): 11.35) as the first eluting peak, and 2-(4-(((1R,2S,4S)-7-oxabicyclo[2.2.1]heptan-2-yl)amino)pyrido[3,4-d]pyridazin-1-yl)-5-(trifluoromethyl) phenol (Compound 10B*) (2.9 mg. 2.5% yield; RT (min): 15.39) as the second eluting peak. Stereochemistry was arbitrarily assigned.

Compound 10B*: LCMS: (ES, m/z): RT=1.26 min, m/z=403.0 [M+H]; 1H NMR (400 MHZ, Methanol-d₄) δ 9.80 (d, J=1.0 Hz, 1H), 8.88 (d, J=5.6 Hz, 1H), 7.59 (d, J=7.8 Hz, 1H), 7.54-7.48 (m, 1H), 7.37-7.30 (m, 1H), 7.28 (d, J=1.7 Hz, 1H), 5.17 (t, J=4.9 Hz, 1H), 4.65 (q, J=3.7, 2.7 Hz, 1H), 4.58-4.48 (m, 1H), 2.45-2.33 (m, 1H), 2.09-1.98 (m. 1H), 1.85-1.77 (m, 2H), 1.77-1.68 (m, 1H), 1.68-1.57 (m, 1H).

Compound 10D*: LCMS: (ES, m/z): RT=1.25 min, m/z=403.0 [M+H]: ¹H NMR (400 MHZ, Methanol-d₄) δ 9.81 (d, J=1.0 Hz, 1H), 8.88 (d, J=5.7 Hz, 1H), 7.59 (d, J=7.9 Hz, 1H), 7.51 (dd, J=5.7, 1.0 Hz, 1H), 7.37-7.30 (m, 1H), 7.28 (d, J=2.0 Hz, 1H), 5.17 (t, J=4.9 Hz, 1H), 4.69-4.62 (m, 1H), 4.58-4.48 (m, 1H), 2.45-2.33 (m, 1H), 2.09-1.98 (m, 1H), 1.85-1.76 (m, 2H), 1.80-1.68 (m. 1H), 1.68-1.57 (m, 1H).

Compounds provided in the below Table C were synthesized or may be synthesized following the above General Synthetic Protocol B and the Protocol B Examples. Dashed lines (--) indicates that no data is available.

TABLE C
Protocol B compounds

| | | LCMS | | |
|---|---|---|---|---|
| | | (m/z) | | |
| # | Compound | [M + 1]⁺ | 1HINMR | Conditions |
| 4B*-r | 2-(4-(((1R,3s,5S)-8- | — | — | Compound 4B* may |
| | oxabicyclo[3.2.1]octan- | | | be synthesized |
| | 3-yl)amino)pyrido[3,4- | | | following Protocol B |
| | d]pyridazin-1-yl)-5- | | | and Examples 7-8 |
| | (trifluoromethyl)phenol | | | using |
| | | | | (1R,3s,5S)-8- |
| | | | | oxabicyclo[3.2.1]octan- |
| | | | | 3-amine (Example |
| | | | | 3 intermediate) as the |
| | | | | step 1 amine (i) |
| | | | | reagent. |
| | | | | Stereochemistry may |
| | | | | be rationally assigned |
| | | | | based on comparison |
| | | | | to Compound 4A*-r |
| | | | | of Example 7 and use |
| | | | | of chiral starting |
| | | | | material. |
| 11 | 2-(4-((8- | — | — | |
| | oxabicyclo[3.2.1]octan- | | | |
| | 3-yl)amino)pyrido[3,4- | | | |
| | d]pyridazin-1-yl)-5- | | | |
| | (difluoromethyl)phenol | | | |
| 11A*-r | 2-(4-(((1R,3r,5S)-8- | 399.05 | ¹H NMR (400 MHz, | Compound 11A*-r |
| | oxabicyclo[3.2.1]octan- | | DMSO-d₆) δ 10.16 (s, | was synthesized |
| | 3-yl)amino)pyrido[3,4- | | 1H), 9.73 (s, 1H), 8.87 (s, | following Protocol B |
| | d]pyridazin-1-yl)-5- | | 1H), 7.51-7.45 (m, 2H), | and Example 7 using |
| | (difluoromethyl)phenol | | 7.31 (d, J = 5.6 Hz, 1H), | (1R,3r,5S)-3- |
| | | | 7.24-7.08 (m, 3H), 4.37 | oxabicyclo[3.2. 1]octan- |
| | | | (d, J = 14.8 Hz, 3H), 2.27- | 8-amine as the step |
| | | | 2.12 (m, 4H), 2.05 (d, J = | 1 amine (i) reagent. |
| | | | 14.4 Hz, 2H), 1.88 (d, J = | Stereochemistry of |
| | | | 9,2 Hz, 2H). | product was |
| 11B*-r | 2-(4-(((1R,3s,5S)-8- | — | — | rationally assigned |
| | oxabicyclo[3.2.1]octan- | | | based on use of chiral |
| | 3-yl)amino)pyrido[3,4- | | | starting material. |
| | d]pyridazin-1-yl)-5- | | | Method X |
| | (difluoromethyl)phenol | | | RT = 0.99 min |
| | | | | Compound 11B*-r |
| | | | | may be synthesized |
| | | | | following Protocol B |
| | | | | and Example 7 using |
| | | | | (1R,3s,5S)-3- |
| | | | | oxabicyclo[3.2. 1]octan- |
| | | | | 8-amine (Example |
| | | | | 3 intermediate) as the |
| | | | | step 1 amine (i) |
| | | | | reagent. |
| | | | | Stereochemistry of |
| | | | | product was |
| | | | | rationally assigned |
| | | | | based on use of chiral |
| | | | | starting material. |
| 12 | 6-(4-((8- | — | — | Compound 12A*-r |
| | oxabicyclo[3.2.1]octan- | | | was synthesized |
| | 3-yl)amino)pyrido[3,4- | | | following Protocol B |
| | d]pyridazin-1-yl)-2- | | | and Example 7 using |
| | fluoro-3-methylphenol | | | (1R,3r,5S)-3- |
| | | | | oxabicyclo[3.2.1]octan- |
| | | | | 8-amine as the step |
| | | | | 1 amine (i) reagent. |
| 12A*-r | 6-(4-(((1R,3r,5S)-8- | 381.15 | ¹H NMR (400 MHz, | Stereochemistry of |
| | oxabicyclo[3.2.1]octan- | | DMSO-d₆) δ 9.72 (d, J = | product was |
| | 3-yl)amino)pyrido[3,4- | | 0.9 Hz, 2H), 8.87 (d, J = | rationally assigned |
| | d]pyridazin-1-yl)-2- | | 5.6 Hz, 1H), 7.47 (d. J = | based on use of chiral |
| | fluoro-3-methylphenol | | 3.4 Hz, 1H), 7.35(d, J = | starting material. |
| | | | 5.6 Hz, 1H), 7.07 (d, J = | Method Y |
| | | | 8.0 Hz, 1H), 6.90-6.84 (t, | RT(min): 10.44. |
| | | | 1H), 4.38-4.32 (m, 3H), | Compound 12B*-r |
| | | | 2.32 (d, J = 2.1 Hz, 3H), | may be synthesized |
| | | | 2.28-2.12 (m, 4H), 2.10- | following Protocol B |
| | | | 2.01 (m, 2H), 1.91- | and Examples 7-8 |
| | | | 1.83 (m, 2H). | using (1R,3s,5S)-3- |
| 12B*-r | 6-(4-(((1R,3s,5S)-8- | — | — | oxabicyclo[3.2.1]octan- |
| | oxabicyclo[3.2.1]octan- | | | 8-amine (Example |
| | 3-yl)amino)pyrido[3,4- | | | 3 intermediate) as the |
| | d]pyridazin-1-yl)-2- | | | step 1 amine (i) |
| | fluoro-3-methylphenol | | | reagent. |
| | | | | Stereochemistry of |
| | | | | product may be |
| | | | | rationally assigned |
| | | | | based on use of chiral |
| | | | | starting material. |
| 13 | 2-(4-((3- | — | — | Compound 13A was |
| | oxabicyclo[3.2.1]octan- | | | synthesized following |
| | 8-yl)amino)pyrido[3,4- | | | Protocol B and |
| | d]pyridazin-1-yl)-5- | | | Example 8 using |
| | (trifluoromethyl)phenol | | | (1R,5S,8s)-3- |
| 13A | 2-(4-(((1R,5S,8s)-3- | 417.10 | ¹H NMR (400 MHz, | oxabicyclo[3.2.1]octan- |
| | oxabicyclo[3.2.1]octan- | | DMSO-d₆) δ 9.97 (s, 1H), | 8-amine (from |
| | 8-yl)amino)pyrido[3,4- | | 8.88 (d, J = 5.6 Hz, 1H), | Example 4, step 1; |
| | d]pyridazin-1-yl)-5- | | 7.87 (d, J = 3.5 Hz, 1H), | stereochemistry |
| | (trifluoromethyl)phenol | | 7.57 (d, J = 7.8 Hz, 1H), | retroactively assigned |
| | | | 7.35-7.28 (m, 3H), 4.16- | based on absolute |
| | | | 4.03 (m, 3H), 3.37 (dd, J = | stereochemical |
| | | | 11.4, 2.7 Hz, 2H), 2.47 | determination of |
| | | | (d, J = 4.9 Hz, 2H), 1.92- | Compound 6A as the |
| | | | 1.78 (m, 4H). | step 1 amine (i) |
| | | | | reagent. |
| 13B | 2-(4-(((1R,5S,8r)-3- | — | — | Method EE |
| | oxabicyclo[3.2.1]octan- | | | RT(min): 19.0 |
| | 8-yl)amino)pyrido[3,4- | | | Compound 13B may |
| | d]pyridazin-1-yl)-5- | | | be synthesized |
| | (trifluoromethyl)phenol | | | following Protocol B |
| | | | | and Examples 7-8 |
| | | | | using (1R,5S,8r)-3- |
| | | | | oxabicyclo[3.2.1]octan- |
| | | | | 8-amine as the step |
| | | | | 1 amine (i) reagent. |
| | | | | The amine reagent |
| | | | | may be obtained from |
| | | | | a synthetic route |
| | | | | similar to what is |
| | | | | used for Example 3: |
| | | | | formation of a |
| | | | | mesylate from 3- |
| | | | | oxabicyclo[3.2.1]octan- |
| | | | | 8-ol obtained in |
| | | | | Example 8, then |
| | | | | displacement with an |
| | | | | azide and reduction to |
| | | | | the primary amine. |
| | | | | Stereochemistry may |
| | | | | be retroactively |
| | | | | assigned based on |
| | | | | absolute |
| | | | | stercochemical |
| | | | | determination of |
| | | | | Compound 6A. |
| 14 | 2-(4-((3- | — | — | Compound 14A was |
| | oxabicyclo[3.2.1]octan- | | | synthesized following |
| | 8-yl)amino)pyrido[3,4- | | | Protocol B and |
| | d]pyridazin-1-yl)-5- | | | Example 8 using |
| | (difluoromethyl)phenol | | | (1R,5S,8s)-3- |
| 14A | 2-(4-(((1R,5S,8s)-3- | 399.15 | ¹H NMR (400 MHz, | oxabicyclo[3.2. 1]octan- |
| | oxabicyclo[3.2.1]octan- | | DMSO-d₆) δ 8.99 (d, J = | 8-amine (from |
| | 8-yl)amino)pyrido[3,4- | | 5.6 Hz, 1H), 8.87 (s, 1H), | Example 4, step 1; |
| | d]pyridazin-1-yl)-5- | | 8.51 (d, J = 5.7 Hz, 1H), | stereochemistry |
| | (difluoromethyl)phenol | | 7.63 (s, 1H), 7.53 (d, J = | retroactively assigned |
| | | | 7.8 Hz, 1H), 7.19 (d, J = | based on absolute |
| | | | 8.2 Hz, 2H), 7.09 (t, J = | stereochemical |
| | | | 56.1 Hz, 1H), 4.07 (d, J = | determination of |
| | | | 11.5 Hz, 3H), 3.37 (d, J = | Compound 6A) as |
| | | | 10.8 Hz, 2H), 2.47 (s, 2H), | the step 1 amine (i) |
| | | | 1.85 (g, J = 9.9, 9.0 Hz, | reagent. |
| | | | 4H). | Method FF |
| 14B | 2-(4-(((1R,5S,8r)-3- | — | — | RT(min): 13.55 |
| | oxabicyclo[3.2.1]octan- | | | Compound 14B may |
| | 8-yl)amino)pyrido[3,4- | | | be synthesized |
| | d]pyridazin-1-yl)-5- | | | following Protocol B |
| | (difluoromethyl)phenol | | | and Examples 7-8 |
| | | | | using (1R,5S,8r)-3- |
| | | | | oxabicyclo[3.2.1]octan- |
| | | | | 8-amine as the step |
| | | | | 1 amine (1) reagent. |
| | | | | The amine reagent |
| | | | | may be obtained from |
| | | | | a synthetic route |
| | | | | similar to what is |
| | | | | used for Example 3: |
| | | | | formation of a |
| | | | | mesylate from 3- |
| | | | | oxabicyclo[3.2.1]octan- |
| | | | | 8-ol obtained in |
| | | | | Example 8, then |
| | | | | displacement with an |
| | | | | azide and reduction to |
| | | | | the primary amine. |
| | | | | Stereochemistry may |
| | | | | be retroactively |
| | | | | assigned based on |
| | | | | absolute |
| | | | | stereochemical |
| | | | | determination of |
| | | | | Compound 6A. |
| 15 | 6-(4-((3- | — | — | Compound 15B may |
| | oxabicyclo[3.2.1]octan- | | | be synthesized |
| | 8-yl)amino)pyrido[3,4- | | | following Protocol B |
| | d]pyridazin-1-yl)-2- | | | and Examples 7-8 |
| | fluoro-3-methylphenol | | | using (1R,5S,8r)-3- |
| 15B | 6-(4-(((1R,5S,8r)-3- | — | — | oxabicyclo[3.2.1]octan- |
| | oxabicyclo[3.2.1]octan- | | | 8-amine as the step |
| | 8-yl)amino)pyrido[3,4- | | | 1 amine (1) reagent. |
| | d]pyridazin-1-yl)-2- | | | The amine reagent |
| | fluoro-3-methylphenol | | | may be obtained from |
| | | | | a synthetic route |
| | | | | similar to what is |
| | | | | used for Example 3: |
| | | | | formation of a |
| | | | | mesylate from 3- |
| | | | | oxabicyclo[3.2.1]octan- |
| | | | | 8-ol obtained in |
| | | | | Example 8, then |
| | | | | displacement with an |
| | | | | azide and reduction to |
| | | | | the primary amine. |
| | | | | Stereochemistry |
| | | | | retroactively may be |
| | | | | assigned based on |
| | | | | absolute |
| | | | | stereochemical |
| | | | | determination of |
| | | | | Compound 6A. |
| 16 | 2-(4-((3- | — | — | Compound 16B may |
| | oxabicyclo[3.2.1]octan- | | | be synthesized |
| | 8-yl)amino)pyrido[3,4- | | | following Protocol B |
| | d]pyridazin-1-yl)-3- | | | and Examples 7-11 |
| | fluoro-5-methylphenol | | | using (1R,5S,8r)-3- |
| | | | | oxabicyclo[3.2.1]octan- |
| 16B | 2-(4-(((1R,5S,8r)-3- | — | — | 8-amine as the step |
| | oxabicyclo[3.2.1]octan- | | | 1 amine (i) reagent. |
| | 8-yl)amino)pyrido[3,4- | | | The amine reagent |
| | d]pyridazin-1-yl)-3- | | | may be obtained from |
| | fluoro-5-methylphenol | | | a synthetic route |
| | | | | similar to what is |
| | | | | used for Example 3: |
| | | | | formation of a |
| | | | | mesylate from 3- |
| | | | | oxabicyclo[3.2.1]octan- |
| | | | | 8-ol obtained in |
| | | | | Example 9, then |
| | | | | displacement with an |
| | | | | azide and reduction to |
| | | | | the primary amine. |
| | | | | Stereochemistry may |
| | | | | be retroactively |
| | | | | assigned based on |
| | | | | absolute |
| | | | | stereochemical |
| | | | | determination of |
| | | | | Compound 6A. |
| 17 | 2-(4-((3- | — | — | Compound 17B may |
| | oxabicyclo[3.2.1]octan- | | | be synthesized |
| | 8-yl)amino)pyrido[3,4- | | | following Protocol B |
| | dipyridazin-1-yl)-3,5- | | | and Examples 7-11 |
| | dimethylphenol | | | using (1R,5S,8r)-3- |
| 17B | 2-(4-(((1R,5S,8r)-3- | — | — | oxabicyclo[3.2.1]octan- |
| | oxabicyclo[3.2.1]octan- | | | 8-amine as the step |
| | 8-yl)amino)pyrido[3,4- | | | I amine (i) reagent. |
| | d]pyridazin-1-yl)-3,5- | | | The amine reagent |
| | dimethylphenol | | | may be obtained from |
| | | | | a synthetic route |
| | | | | similar to what is |
| | | | | used for Example 3: |
| | | | | formation of a |
| | | | | mesylate from 3- |
| | | | | oxabicyclo[3.2.1]octan- |
| | | | | 8-ol obtained in |
| | | | | Example 10, then |
| | | | | displacement with an |
| | | | | azide and reduction to |
| | | | | the primary amine. |
| | | | | Stereochemistry may |
| | | | | be retroactively |
| | | | | assigned based on |
| | | | | absolute |
| | | | | stereochemical |
| | | | | determination of |
| | | | | Compound 6A. |
| 18 | 4-(4-((3- | — | — | Compound 18B may |
| | oxabicyclo[3.2.1]octan- | | | be synthesized |
| | 8-yl)amino)pyrido[3,4- | | | following Protocol B |
| | d]pyridazin-1-yl)-3- | | | and Examples 7-11 |
| | hydroxybenzonitrile | | | using (1R,5S,8r)-3- |
| 18B | 4-(4-(((1R,5S,8r)-3- | — | — | oxabicyclo[3.2.1]octan- |
| | oxabicyclo[3.2.1]octan- | | | 8-amine as the step |
| | 8-yl)amino)pyrido[3,4- | | | 1 amine (i) reagent. |
| | d]pyridazin-1-yl)-3- | | | The amine reagent |
| | hydroxybenzonitrile | | | may be obtained from |
| | | | | a synthetic route |
| | | | | similar to what is |
| | | | | used for Example 3: |
| | | | | formation of a |
| | | | | mesylate from 3- |
| | | | | oxabicyclo[3.2.1]octan- |
| | | | | 8-ol obtained in |
| | | | | Example 10, then |
| | | | | displacement with an |
| | | | | azide and reduction to |
| | | | | the primary amine. |
| | | | | Stereochemistry may |
| | | | | be retroactively |
| | | | | assigned based on |
| | | | | absolute |
| | | | | stereochemical |
| | | | | determination of |
| | | | | Compound 6A. |

### Assay Methods

The biological activity, brain penetrance, stability, and/or solubility of the compounds of the present disclosure may be determined utilizing the assays described herein.

### (i) Human PBMC NLRP3 Assay

The objective of this assay to is demonstrate if a test compound is able to interfere with human NLRP3 function in a cellular system.

Reagents: Human PBMCs (Normal): iXCells Cat #10HU-003; RPMI 1640 medium with GlutaMAX: ThermoFisher Cat #61870127 (Complete Media: 4.5 g/L D-glucose, 10% FBS, 100 mM NaPyr, 1% Pen/Strep, 10 mM HEPES and 0.05 mM of @-mercaptoethanol; Assay Media: 4.5 g/L D-glucose, 100 mM NaPyr, 1% Pen/Strep. 10 mM HEPES and 0.05 mM of mercaptoethanol; 96-well V-bottom Plates: Costar Cat #3894; LPS (E. coli 026:B6): Sigma Cat #L2654, stock 5 mg/mL in PBS; ATP: Sigma Cat #A6419, prepared in a 250 mM stock in 1 M HEPES (adjusted to pH 7.4).

Cryopreserved PBMCs are rapidly thawed in a 37° C. water bath for 2 min. Cells are then centrifuged at 1200 RPM for 5 min and resuspended in ˜50 mL of fresh RPMI 1640 Complete Medium. A count is undertaken using a hemocytometer and adjusted to 2.5×10⁵ cells/mL. V-shaped 96-well plates are seeded with 200 μL of PMBCs (5×10⁴) per well and subsequently incubated overnight at 37° C. with 5% CO₂. Assay Media is then prepared containing 100 ng/ml of LPS. PBMCs are then centrifuged at 1,200 RPM for 5 min, serum containing media was aspirated, and 150 μL/well of Assay Media+LPS is immediately added. Assay Media without LPS is added in the untreated control wells. Cells are then primed with LPS for 4 h at 37° C. with 5% CO₂. A concentration response curve (CRC) is prepared of 1000× test compound in 100% dimethylsulfoxide (DMSO). The CRC is then diluted 1:50 in Assay Media and then further diluted by 1:5× in Assay Media resulting in a final 4× CRC in 0.4% DMSO/Assay Media. 50 μL/well of 4× test compound CRC or vehicle (0.4% DMSO/Assay Media) is then transferred into each well and subsequently incubated for 30 min at 37° C. with 5% CO₂. Cells are stimulated by adding 4 μL of 250 mM ATP, using a 250 mM stock prepared in 1 M Hepes without further dilution (for a final concentration of 5 mM), for 1 h at 37° C. with 5% CO₂ Plates are then centrifuged at 1200 RPM for 5 min; and 50 μl of the media is transferred to a clean 96-well storage plate for cytokine measurements using the mesoscale platform, and stored at −80° C. until analyzed. Compounds are added 30 minutes before priming the cells with LPS when TNFα was required to be quantified in the cytokine panel to confirm selectivity.

(ii) Human Whole Blood (hWB) NLRP3 Assay

The objective of this assay to is demonstrate if a test compound is able to interfere with human NLRP3 function in a whole blood system.

Human whole blood is drawn from healthy volunteers after obtaining written informed consent. Heparin lithium coated tubes are used to collect blood from volunteers. Blood samples are distributed on 96 well plates using 90 μl per well. Priming is performed by adding 5 μl of LPS (026: B6; Sigma L-2654) at a final concentration of 1 μg/ml for 4.5 hours in a humidified incubator with 37° C. 5% CO₂. Thirty minutes prior to NLRP3 activation, 5 μl of a 20× compound solution or vehicle (2% dimethylsulfoxide (DMSO)) is added to each well and plates were incubated on a shaker (450 rpm) in a humidified incubator with 37° C., 5% CO₂. Activation is then performed by adding 3.3 μl of a 31× ATP solution per well. At the end of the 30 minutes stimulation, the plates are centrifuged (800 g, 10 min, room temperature) and the plasma from each well is frozen at −80° C. IL-1β levels in the supernatant were analyzed using a mesoscale discovery assay (MSD K151TUK) according to the manufacturers’ instructions.

(iii) Mouse Whole Blood (mWB) NLRP3 Assay

The objective of this assay to is demonstrate if a test compound is able to interfere with mouse NLRP3 function in a whole blood system.

Reagents: The following reagents are used: blood collection tubes (Heparin); U-bottom 96-well tissue culture (Falcon 353077): HBSS for LPS, ATP and compound dilutions (Gibco 24020-117); and LPS, E. coli serotype 026: B6 (Sigma L-2654).

Mouse IL-1b MSD assay: Blood is drawn from female CDI mice (9 to 10 weeks) by cardiac puncture Blood is plated (135 μL) per well in 96-well U-bottom plates. 7.5 μL of 20× LPS (20 g/mL. final concentration of 1 μg/mL) is added and mixed by gentle pipetting, and the mixture is incubated in a TC incubator for five hours. 7.5 μL of 20× compound or vehicle per well is added and mixed by gentle pipetting. Compounds are diluted 1/50 in HBSS to prepare a 20× dilution curve in 2% dimethylsulfoxide (DMSO). The mixture is incubated for 30 minutes in a TC incubator with shaking (450 rpm). 5 μl of 31× ATP (155 mM, final concentration of 5 mM) is added and mixed by gentle pipetting. The mixture is incubated in a TC incubator for 30 minutes with shaking (450 rpm). The plate is centrifuged for 10 minutes at 800×g and ˜70 μL of plasma was removed. Plasma is frozen if required. Mouse IL-1b is analyzed with IL-1b MSD assay (K152TUK).

Compounds are added 30 minutes before priming the cells with LPS when TNFα was required to be quantified in the cytokine panel to confirm selectivity.

### (iv) Brain Levels (Kp and Kpu,u)—NLRP3

The in vivo total brain to plasma ratio was assessed in CDI mice after oral administration of the compound. Blood was collected at several timepoints up to 24 h, and transferred into plastic micro centrifuge tubes with EDTA-K2 as anticoagulant. Blood samples were centrifuged at 4000 g for 5 minutes at 4° C., to obtain plasma, then immediately frozen and stored at −75±15° C., prior to analysis. Animals were terminally anaesthetized with a rising concentration of CO2 gas at about 1 minute. At selected timepoints, whole brains were removed from the skull, rinsed in cold PBS to remove blood, dried with clean gauze, then frozen in liquid nitrogen and kept at −75±15° C. before analysis. At the time of analysis, brain samples were homogenized with PBS by tissue weight (g) to PBS volume (mL) ratio 1:3.

Plasma and brain drug levels were quantified by LC/MS/MS on an AB Sciex Triple Quad 5500+instrument, after separation on a HALO 160 A ES-C18, 2.7 μm 2.1×50 mm column. Quantitation was performed using a calibration curve prepared in blank plasma or blank brain homogenate. The software WinNonlin (Phoenix™) was used for pharmacokinetic analysis from the concentrations versus time data, including the AUC_{inf} and AUCₗₐₛₜ. The Kp ratio (total brain concentration over total plasma concentration) was calculated as (AUC_{tot,br})/(AUC_{tot,pl}).

The unbound brain exposure was assessed as Kpᵤᵤ, which is the free brain/free plasma concentration ratio (C_{u,bt}/C_{u,pl}). The C_{u,br}/C_{u,pl} ratios were obtained from in vivo total brain to plasma ratios (C_{tot,br}/C_{tot,pl}) by using in vitro determined F_{u,br} and F_{u,pl}.

Plasma protein binding and brain homogenate protein binding were measured by equilibrium dialysis in a HTDialysis plate. The dialysis membranes were soaked in ultrapure water for 60 minutes to separate strips, then in 20% ethanol for 20 minutes, finally in dialysis buffer for 20 minutes. The dialysis set up was assembled according to the manufacturer's instruction. Each cell received 150 μL of plasma or brain homogenate spiked with 1 mM of compound, and dialyzed against an equal volume of dialysis buffer (PBS). The dialysis plate was sealed and incubated in an incubator at 37° C. with 5% CO2 at 100 rpm for 6 hours. At the end of incubation, compound concentration was measured in both chambers by LC-MS/MS on a Triple Quad™ 5500 from AB Inc after separation on a XSelect Hss T3.2.5μ (2.1×30 mm) column. Free fraction (Fu) was calculated as (Peak Area Ratio buffer chamber/Peak Area Ratio plasma chamber). Kpᵤᵤ=(AUC_{tot,br}×F_{u,br})/(AUC_{tot,br}×F_{u,pl})

The kp values were calculated by measuring whole brain drug levels over 24 h (AUC) in mice dosed at 20 mpk PO, and dividing by plasma AUC. The Kpu,u was then calculated upon correcting this kp value with mouse plasma protein binding and mouse brain homogenates binding.

A Kpu,u value >0.3 is considered brain penetrant, and a Kpu,u value ≤0.3 is not considered brain penetrant. When the Kpu,u value is not determined, the Kp value may be useful as a metric of potential brain penetrance if the Kp value is >0.3.

### (v) Mouse and Human Hepatocyte Stability Assays

A hepatocyte stability assay is a laboratory-based method used to determine the metabolic stability of a compound in hepatocytes (liver cells). This assay provides valuable information about how quickly a drug is metabolized in the liver and can be used to assess its potential effectiveness and safety in drug discovery.

In the assay, cither human or mouse hepatocytes are incubated with the test compound at a controlled temperature of 37° C., for different time periods (e.g., 5, 15, 30, 60, and 120 minutes). At each time point during the incubation, samples are taken, the reaction is terminated, and the amount of test compound remaining analyzed using LC-MS/MS to monitor the disappearance of the test compound over time (Gradient). From these data, a half-life can be calculated (t ¼=time it takes for ½ of the test compound to be consumed in the hepatocyte incubation). See, e.g., Coe et al., Methods in Pharmacology & Toxicology (2008) 151.

### (vi) Solubility Protocol in Phosphate Buffered Saline (PBS)

The kinetic solubility of test compounds and control compounds was measured in commercial phosphate buffered saline (PBS), pH 7.4 (Wisent, Canada).

Briefly, a stock solution (20 mM DMSO for controls, and 10 mM DMSO for test compounds) was combined with PBS buffer to reach a targeted concentration of 400 μM for controls and 200 μM for test compounds. The spiked-PBS mixtures were then agitated on a VX-2500 multi-tube vortexer (VWR) for 2 hours at room temperature (18° C.). Following agitation, the samples were filtered on a glass fiber filter (1 μm) and the eluates were diluted 400-fold with a mixture of acetonitrile:water (1:1). Solubility determination was then performed against one standard sample prepared in high organic content at the expected top concentration. The lower limit of quantification was arbitrarily set at 1 μM (400-fold dilution of the top concentration) for assay controls, and 0.5 μM for test compounds. On each experimental run, nicardipine and imipramine were assessed as reference compounds for low and high solubility, respectively. All samples were assessed in triplicate and analyzed by LC-MS/MS (using a CTC PAL autosampler, Thermo Accela UPLC, and a Thermo Quantum mass spectrometer) using electrospray ionization against standards prepared in the same matrix

(vii) Results

Test data for certain compounds described herein via one or more of the above described assays is provided in Table D. Dashed (--) lines indicate not determined

TABLE D
Activity Data

| | | | | | Hep | Hep |
|---|---|---|---|---|---|---|
| | hWB | | | Sol | (mouse) | (human) |
| Comp | (uM) | | | (PBS) | (t½ | (t½ |
| No. | IC₅₀ | Kp | Kpu, u | uM | min) | min) |
| 1A*-r | 1.39 | 0.047 | — | 3.5 | — | — |
| 1B*-r | — | — | — | — | — | — |
| 2A*-1 | 0.33 | 0.27 | 0.16 | 1.9 | 71 | >480 |
| 2B*-r | 4.12 | — | — | — | 220 | — |
| 3A* | 1.16 | 0.30 | 0.15 | >300 | 130 | >240 |
| 3B* | 5.0 | — | — | — | — | — |
| 3C* | 0.27 | — | — | — | 100 | — |
| 3D* | 2.42 | — | — | — | 10 | — |
| 4A*-r | 1.1 | 0.13 | 0.09 | 2.4 | 200 | >480 |
| 4B*-r | — | — | — | — | — | — |
| 5A | 0.15 | 0.87 | 0.44 | 25 | 160 | >240 |
| 5B | — | — | — | — | — | — |
| 6A | 0.20 | 0.76 | 0.51 | >300 | 160 | >240 |
| 6B | — | — | — | — | — | — |
| 7A* | 0.23 | 0.61 | — | — | 57 | >480 |
| 7B* | 5.30 | — | — | — | — | — |
| 7C* | 0.11 | — | — | — | 21 | 280 |
| 7D* | 4.66 | — | — | — | — | — |
| 8A* | 0.27 | — | — | — | 250 | — |
| 8B* | 0.59 | 0.35 | 0.10 | >300 | 280 | — |
| 9A* | >50 | — | — | — | — | — |
| 9B* | >50 | — | — | — | — | — |
| 10A* | 0.92 | 0.46 | 0.49 | 2.1 | 110 | 190 |
| 10B* | 6.76 | — | — | — | — | — |
| 10C* | 0.81 | 0.48 | 0.21 | — | 140 | 470 |
| 10D* | 5.34 | — | — | — | 480 | >480 |
| 11A*-r | 0.81 | 0.08 | 0.06 | 0.7 | 230 | >240 |
| 11B*-r | — | — | — | — | — | — |
| 12A*-r | 2.0 | — | — | — | 200 | — |
| 12B*-r | — | — | — | — | — | — |
| 13A | 0.34 | 0.10 | 0.08 | 1.4 | 100 | 290 |
| 13B | — | — | — | — | — | — |
| 14A | 0.20 | 0.37 | 0.20 | 11 | 120 | >480 |
| 14B | — | — | — | — | — | — |
| 15A | 0.34 | 0.41 | 0.45 | >300 | 29 | >480 |
| 15B | — | — | — | — | — | — |
| 15A- | 1.51 | — | — | — | — | — |
| regioisomer | — | — | — | — | — | — |
| 16A | 0.48 | — | — | — | — | — |
| 16B | — | — | — | — | — | — |
| 17A | 0.31 | — | — | — | — | — |
| 17B | — | — | — | — | — | — |
| 18A | 0.32 | — | — | — | — | — |
| 18B | — | — | — | — | — | — |

(viii) Discussion

Compounds of Formula (I) (e.g., compounds of formula (III-a). (IV-a). (V-a). (VI-a), and (VII-a)), have been explored herein as inhibitors of NLRP3.

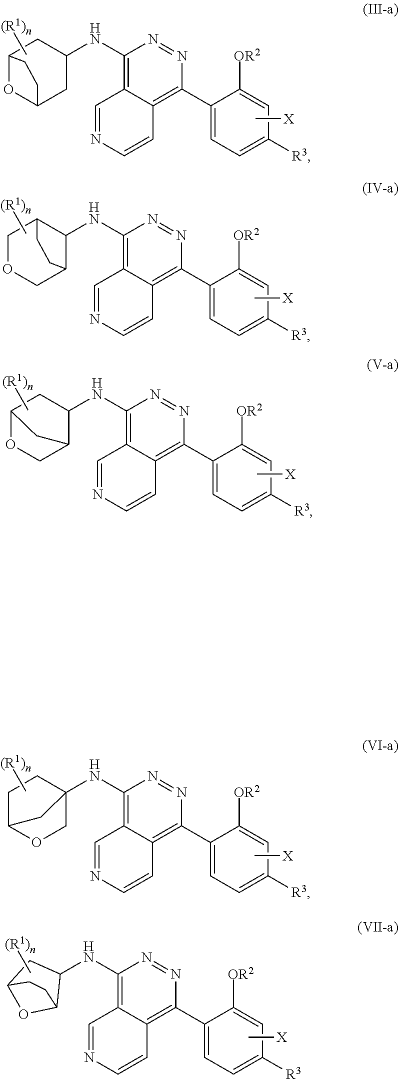

In general, compounds of Formula (III-a), (IV-a), (V-a), and (VII-a) exhibit desirable properties, such as inhibition of NLRP3 activity, in contrast to compounds of Formula (VI-a) which exhibit unexpected inactivity against NLRP3, as shown in above Table D and below Table E1.

TABLE E1

| | | | hWB IC₅₀ |
|---|---|---|---|
| Formula | Compound | No. | (UM) |
| (III-a) (III-b1) | 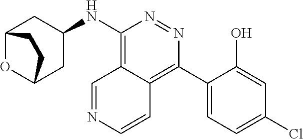 | 1A*-r | 1.39 |
| (IV-a) (IV-c1) | 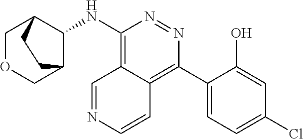 [SMILES: Oc1cc(Cl)ccc1-c1nnc(N[C@H]2[C@@H]3CC[C@H]2COC3)c2cnccc12] | 5A | 0.15 |
| (V-a) | 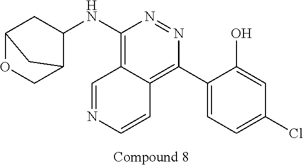 [SMILES: Oc1cc(Cl)ccc1-c1nnc(NC2CC3CC2CO3)c2cnccc12] | 8A* 8B* | 0.27 0.59 |
| (VI-a) | 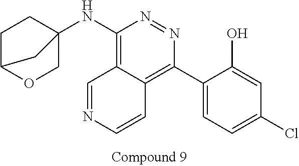 [SMILES: Oc1cc(Cl)ccc1-c1nnc(NC23CCC(C2)OC3)c2cnccc12] | 9A* 9B* | >50 >50 |
| (VII-a) | 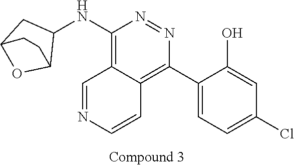 [SMILES: Oc1cc(Cl)ccc1-c1nnc(NC2CC3CCC2O3)c2cnccc12] | 3A* 3B* 3C* 3D* | 1.16 5.0 0.27 2.42 |

Furthermore, it has been confirmed that the specific position of the pyridine ring nitrogen of the pyrido[3,4-d]pyridazine core of compounds of Formula (I) has an improved effect on NLRP3 potency compared to the corresponding pyridine ring nitrogen regioisomer, as shown in above Table D and below Table E2.

TABLE E2

| | | | hWB IC₅₀ |
|---|---|---|---|
| Formula | Compound | No. | (uM) |
| (IV-a) (IV-c1) | 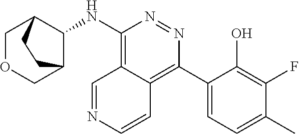 [SMILES: Cc1ccc(-c2nnc(N[C@H]3[C@@H]4CC[C@H]3COC4)c3cnccc23)c(O)c1F] | 15A | 0.34 |
| | 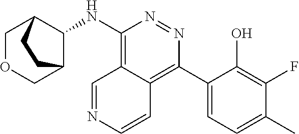 | 15A-regioisomer | 1.51 |

Compounds of Formula (III-a), (IV-a), (V-a), and (VII-a) further demonstrate desirable properties which may be useful in the treatment of systemic (non-central nervous system (non-CNS)) and/or brain penetrant (central nervous system (CNS)) disorders.

For example, compounds of Formula (III-a) may demonstrate a lack of brain penetrance and thus may be useful in the treatment of systemic (non-CNS) NLRP3-mediated disorders. See, e.g., the consistent non-brain penetrant Kp/Kpu,u values across compounds of this series, as shown in above Table D and below Table E3. NLRP3 potency may be improved by replacing the R³ halogen group with an R³ alkyl group. See, e.g., Compound 1A*-r in comparison to Compound 2A*-r. Stability may be improved upon incorporation of an X halogen group. See, e.g., the improvement in mouse hepatocyte stability of Compound 2A*-r in comparison to Compound 12A*-r.

TABLE E3

| | | | | | | | Hep | Hep |
|---|---|---|---|---|---|---|---|---|
| | | | hWB | | | Sol | (mouse) | (human) |
| | | | (uM) | | | (PBS) | (t1/2 | (t1/2 |
| Scaffold | Compound | # | IC₅₀ | Kp | Kpu,u | uM | min) | min) |
| (III-a) (III-b1) | 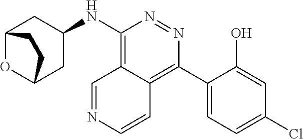 | 1A*-r | 1.39 | 0.047 | — | 3.5 | — | — |
| (III-a) (III-b1) | 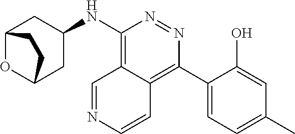 | 2A*-r | 0.33 | 0.27 | 0.16 | 1.9 | 71 | >480 |
| (III-a) (III-b1) | 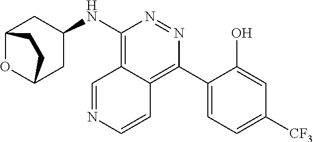 | 4A*-r | 1.1 | 0.13 | 0.09 | 2.4 | 200 | >480 |
| (III-a) (III-b1) | 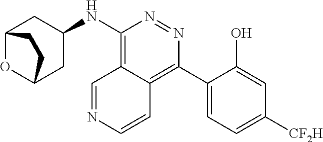 | 11A*-r | 0.81 | 0.08 | 0.06 | 0.7 | 230 | >240 |
| (III-a) (III-b1) | 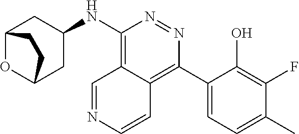 | 12A* | 2.0 | — | — | — | 200 | — |

In contrast, the movement of the bridgehead from 3,5-position (as seen in compounds of Formula (III-a)) to the 2,6 position (as seen in compounds of Formula (IV-a)) provide compounds which may exhibit brain penetrance, and thus may be useful in the treatment of a central nervous system (CNS) NLRP3-mediated disorder. See, e.g., the Kp/Kpu,u values in above Table D and below Table E4. Solubility may be improved by replacing an R³ halogen group with an R³ C₁₋₆ alkyl group. See, e.g., the improved solubility in PBS of Compound 5A in comparison to Compound 6A and Compound 15A.

TABLE E4

| | | | | | | | Hep | Hep |
|---|---|---|---|---|---|---|---|---|
| | | | hWB | | | Sol | (mouse) | (human) |
| | | | (uM) | | | (PBS) | (t1/2 | (t1/2 |
| Scaffold | Compound | # | IC₅₀ | Kp | Kpu,u | uM | min) | min) |
| (IV-a) (IV-c1) | 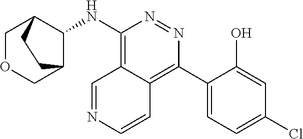 [SMILES: Oc1cc(Cl)ccc1-c1nnc(N[C@H]2[C@@H]3CC[C@H]2COC3)c2cnccc12] | 5A | 0.15 | 0.87 | 0.44 | 25 | 160 | >240 |
| (IV-a) (IV-c1) | 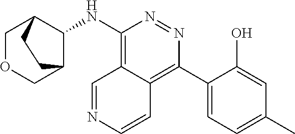 [SMILES: Cc1ccc(-c2nnc(N[C@H]3[C@@H]4CC[C@H]3COC4)c3cnccc23)c(O)c1] | 6A | 0.20 | 0.76 | 0.51 | >300 | 160 | >240 |
| (IV-a) (IV-c1) | 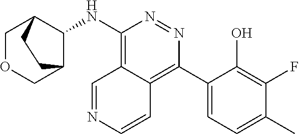 [SMILES: Cc1ccc(-c2nnc(N[C@H]3[C@@H]4CC[C@H]3COC4)c3cnccc23)c(O)c1F] | 15A | 0.34 | 0.41 | 0.45 | >300 | 29 | >480 |
| (IV-a) (IV-c1) | 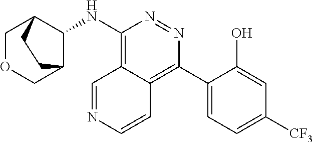 [SMILES: Oc1cc(C(F)(F)F)ccc1-c1nnc(N[C@H]2[C@@H]3CC[C@H]2COC3)c2cnccc12] | 13A | 0.34 | 0.10 | 0.08 | 1.4 | 100 | 290 |
| (IV-a) (IV-c1) | 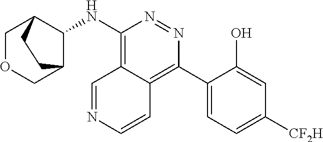 [SMILES: Oc1cc(C(F)F)ccc1-c1nnc(N[C@H]2[C@@H]3CC[C@H]2COC3)c2cnccc12] | 14A | 0.20 | 0.37 | 0.20 | 11 | 120 | >480 |

Moving from a 3,5-bridgehead (of compounds of Formula (III-a)) to a 2,6-bridgehead (of compounds of Formula (IV-a)) or to a 2,5-bridgehead (of compounds of Formula (V-a)) may also lead to an improvement in one or more desirable properties. Compare, for example, the improved NLRP3 potency, brain penetrance and/or solubility moving from Compound 1A*-r of Formula (III-a) to Compound 5A of Formula (IV-a) and the two more NLRP3 potent Compound 8 stereoisomers of Formula (V-a). See, e.g., above Table D and below Table E5.

TABLE E5

| | | | | | | Solubility |
|---|---|---|---|---|---|---|
| | | | hWB | | | in PBS |
| Formula | Compound | # | IC₅₀ | Kp | Kpu,u | (uM) |
| (III-a) (III-b1) | 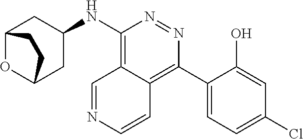 | 1A*-r | 1.39 | 0.047 | — | 3.5 |
| (IV-a) (IV-c1) | 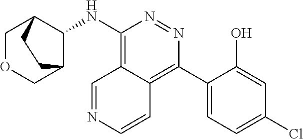 [SMILES: Oc1cc(Cl)ccc1-c1nnc(N[C@H]2[C@@H]3CC[C@H]2COC3)c2cnccc12] | 5A | 0.15 | 0.87 | 0.44 | 25 |
| (V-a) | 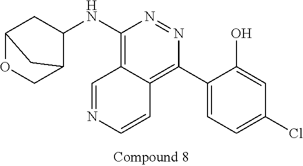 [SMILES: Oc1cc(Cl)ccc1-c1nnc(NC2CC3CC2CO3)c2cnccc12] | 8A* 8B* | 0.27 0.59 | — 0.35 | — 0.10 | — >300 |

Contracting the bridged tetrahydropyranyl ring of compounds of Formula (III-a) to a bridged tetrahydrofuranyl ring of compounds of Formula (VII-a) may also lead to an improvement in one or more desirable properties. See, e.g., above Table D and below Tables E6, E7, and E8 (each listing the two more NLRP3 potent isomers for Compounds 3, 7, and 10, respectively, and comparing to Compound 1A*-r, 2A*-r, and 4A*-r, respectively).

TABLE E6

| | | | | | | Solubility |
|---|---|---|---|---|---|---|
| | | | hWB | | | in PBS |
| Formula | Compound | # | IC₅₀ | Kp | Kpu,u | (uM) |
| (III-a) (III-b1) | 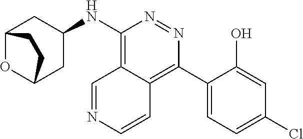 | 1A*-r | 1.39 | 0.047 | — | 3.5 |
| (VII-a) | 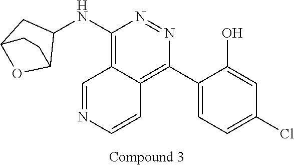 [SMILES: Oc1cc(Cl)ccc1-c1nnc(NC2CC3CCC2O3)c2cnccc12] | 3A* 3C* | 1.16 0.27 | 0.30 — | 0.15 — | >300 — |

TABLE E7

| | | | | | | Solubility |
|---|---|---|---|---|---|---|
| | | | hWB | | | in PBS |
| Formula | Compound | # | IC₅₀ | Kp | Kpu,u | (uM) |
| (III-a) (III-b1) | 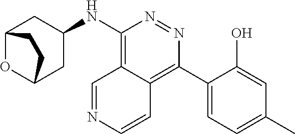 | 2A*-r | 0.33 | 0.27 | 0.16 | 1.9 |
| (VII-a) | 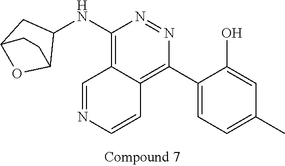 [SMILES: Cc1ccc(-c2nnc(NC3CC4CCC3O4)c3cnccc23)c(O)c1] | 7C* 7A* | 0.11 0.23 | 0.61 — | — — | — — |

TABLE E8

| | | | | | | Solubility |
|---|---|---|---|---|---|---|
| | | | hWB | | | in PBS |
| Formula | Compound | # | IC50 | Kp | Kpu,u | (uM) |
| (III-a) (III-b1) | 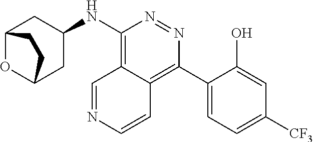 | 4A*-r | 1.1 | 0.13 | 0.09 | 2.4 |
| (VII-a) | 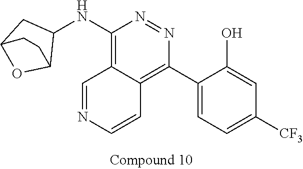 [SMILES: Oc1cc(C(F)(F)F)ccc1-c1nnc(NC2CC3CCC2O3)c2cnccc12] | 10C* 10A* | 0.81 0.92 | 0.48 0.46 | 0.21 0.49 | — 2.1 |

### EQUIVALENTS

The details of one or more embodiments of the disclosure are set forth in the accompanying description above. Although any methods and materials similar or equivalent to those described herein can be used in the practice or testing of the present disclosure, the desired methods and materials are herein described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 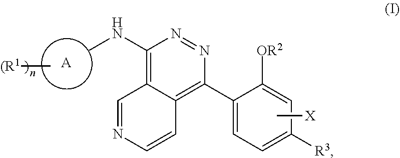
or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen, C₁-C₆ alkyl, or C₁-C₆ alkoxy;
R² is H, C₁-C₆ alkyl, or —C(O)(C₁-C₆ alkyl);
R³ is —OH, halogen, —CN, C₁-C₆ alkyl, or C₁-C₆ alkoxy;
X is H, —OH, halogen, —NH₂, —NH(C₁-C₆ alkyl), —N(C₁-C₆ alkyl)₂, or C₁-C₆ alkyl; and
n is 0, 1, 2, 3, or 4;
wherein each instance of alkyl or alk—is independently and optionally substituted with one or more halogen atoms.

2. The compound of claim 1, or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen, C₁-C₆ alkyl, or C₁-C₆ alkoxy;
R² is H, C₁-C₆ alkyl, or —C(O)(C₁-C₆ alkyl);
R³ is —OH, halogen, C₁-C₆ alkyl, or C₁-C₆ alkoxy;
X is H, —OH, halogen, —NH₂, —NH(C₁-C₆ alkyl), —N(C₁-C₆ alkyl) 2, or C₁-C₈ alkyl; and
n is 0, 1, 2, 3, or 4;
wherein each instance of alkyl or alk—is independently substituted with 0, 1, 2, or 3 halogen atoms.

3. The compound of claim 1, or a pharmaceutically acceptable salt or isotopically labeled derivative thereof, wherein:
A is a 6- to 10-membered bridged bicyclic heterocycloalkyl, wherein the heterocycloalkyl comprises at least one O ring atom;
each R¹ independently is halogen, C₁-C₆ alkyl, or C₁-C₆ alkoxy;
R² is H, C₁-C₆ alkyl, or —C(O)(C₁-C₆ alkyl);
R³ is —CN;
X is H, —OH, halogen, —NH₂, —NH(C₁-C₆ alkyl), —N(C₁-C₆ alkyl) 2, or C₁-C₆ alkyl; and
n is 0, 1, 2, 3, or 4;
wherein each instance of alkyl or alk—is independently substituted with 0, 1, 2, or 3 halogen atoms.

4. The compound of any one of claims 1-3, or a pharmaceutically acceptable salt or isotopically labeled derivative thereof, wherein A is a 6-membered bridged bicyclic heterocycloalkyl comprising one O ring atom.

5. The compound of any one of claims 1-3, or a pharmaceutically acceptable salt or isotopically labeled derivative thereof, wherein A is a 7-membered bridged bicyclic heterocycloalkyl comprising one O ring atom.

6. The compound of any one of claims 1-3, or a pharmaceutically acceptable salt or isotopically labeled derivative thereof, wherein A is an 8-membered bridged bicyclic heterocycloalkyl comprising one O ring atom.

7. The compound of any one of the preceding claims, or a pharmaceutically acceptable salt or isotopically labeled derivative thereof, wherein R² is H.

8. The compound of any one of the preceding claims, or a pharmaceutically acceptable salt or isotopically labeled derivative thereof, wherein X is H or F.

9. The compound of any one of the preceding claims, or a pharmaceutically acceptable salt or isotopically labeled derivative thereof, wherein n is 0.

10. The compound of claim 1, or a pharmaceutically acceptable salt or isotopically labeled derivative thereof, wherein:
A is a 6- to 8-membered bridged bicyclic heterocycloalkyl, wherein the heterocycloalkyl comprises at least one O ring atom;
R² is H;
R³ is halogen, C₁-C₆ haloalkyl, or C₁-C₆ alkyl;
X is H or halogen; and
n is 0.

11. The compound of any one of the preceding claims, or a pharmaceutically acceptable salt or isotopically labeled derivative thereof, wherein R³ is Cl, —CF₃, —CF₂H, or —CH₃.

12. The compound of any one of claims 1-10, or a pharmaceutically acceptable salt or isotopically labeled derivative thereof, wherein R³ is C₁-C₆ haloalkyl or C₁-C₆ alkyl.

13. The compound of claim 12, or a pharmaceutically acceptable salt or isotopically labeled derivative thereof, wherein R³ is —CF₃, —CF₂H, or —CH₃.

14. The compound of claim 1, 3, or 4-9, or a pharmaceutically acceptable salt or isotopically labeled derivative thereof, wherein:
A is a 6- to 8-membered bridged bicyclic heterocycloalkyl, wherein the heterocycloalkyl comprises at least one O ring atom;
R² is H;
R³ is —CN;
X is H or halogen; and
n is 0.

15. The compound of any one of claims 1-14, wherein the compound is of Formula (II-a), (II-b), (II-c), (II-d), (II-e), (II-f), (II-g), (II-h), or (II-i): 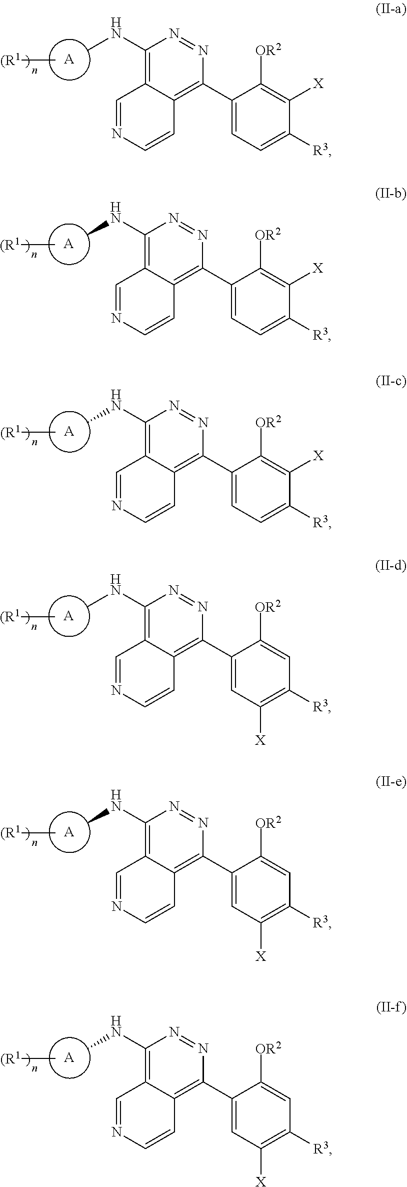 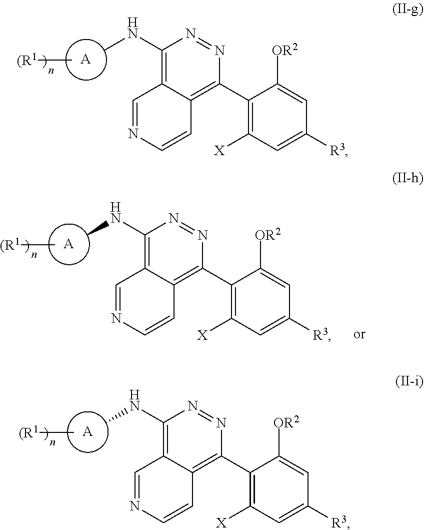
or a pharmaceutically acceptable salt or isotopically labeled derivative thereof.

16. The compound of any one of claims 1-15, wherein the compound is of Formula (III-a), (III-b), or (III-c): 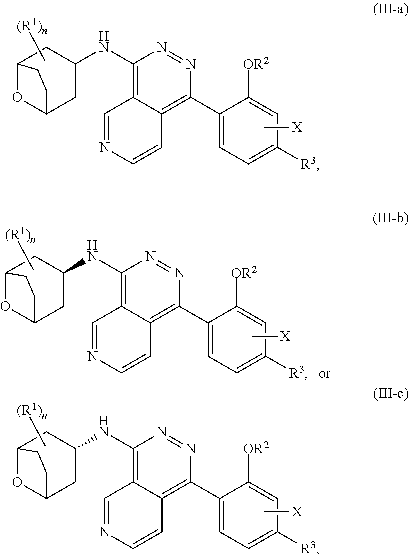
or a pharmaceutically acceptable salt or isotopically labeled derivative thereof.

17. The compound of any one of claims 1-16, wherein the compound is of Formula (III-a1), (III-b1), or (III-c1): 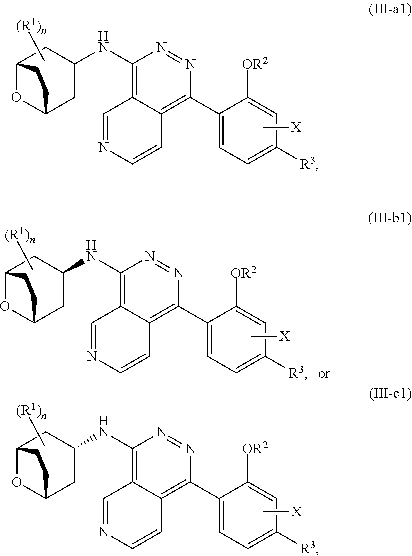
or a pharmaceutically acceptable salt or isotopically labeled derivative thereof.

18. The compound of any one of claims 1-15, wherein the compound is of Formula (IV-a), (IV-b), or (IV-c): 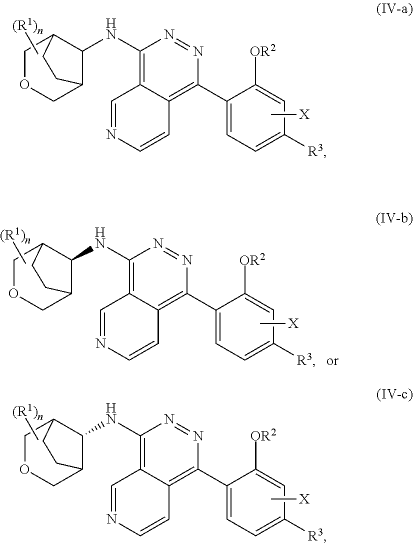
or a pharmaceutically acceptable salt or isotopically labeled derivative thereof.

19. The compound of any one of claims 1-15, wherein the compound is of Formula (IV-a1), (IV-b1), or (IV-c1): 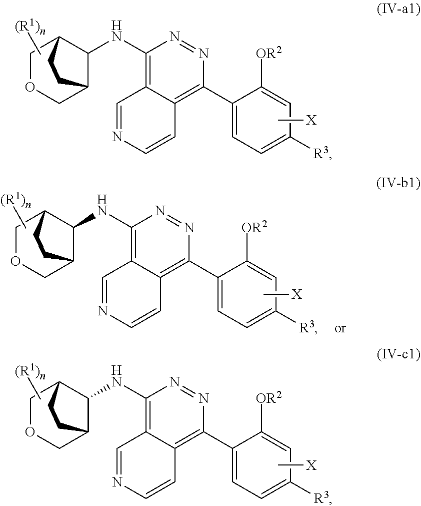
or a pharmaceutically acceptable salt or isotopically labeled derivative thereof.

20. The compound of any one of claims 1-15, wherein the compound is of Formula (V-a), (V-b), or (V-c): 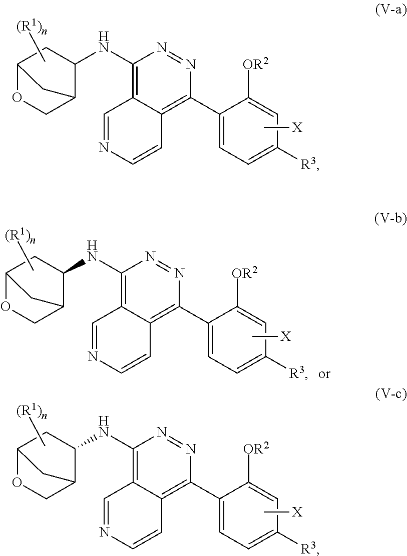
or a pharmaceutically acceptable salt or isotopically labeled derivative thereof.

21. The compound of any one of claims 1-15, wherein the compound is of Formula (V-a1), (V-b1), (V-c1), (V-a2), (V-b2), or (V-€2): 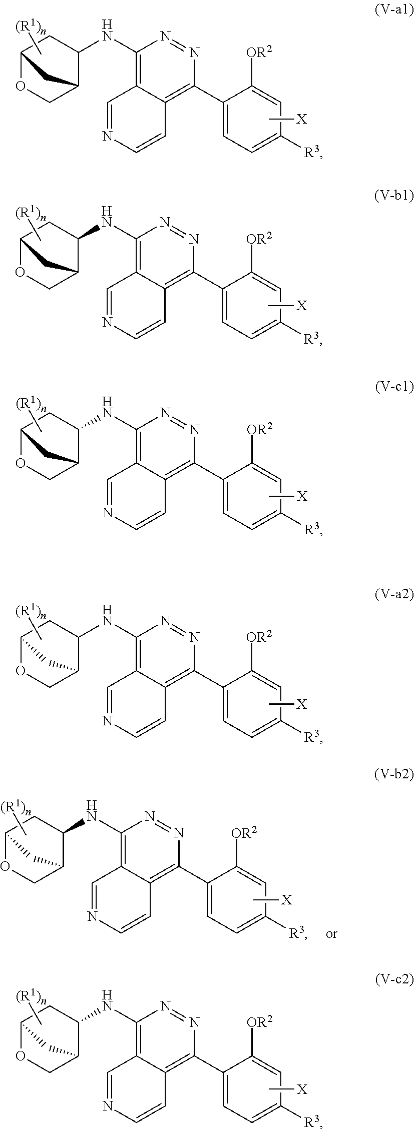
or a pharmaceutically acceptable salt or isotopically labeled derivative thereof.

22. The compound of any one of claims 1-15, wherein the compound is of Formula (VI-a), (VI-b), or (VI-c): 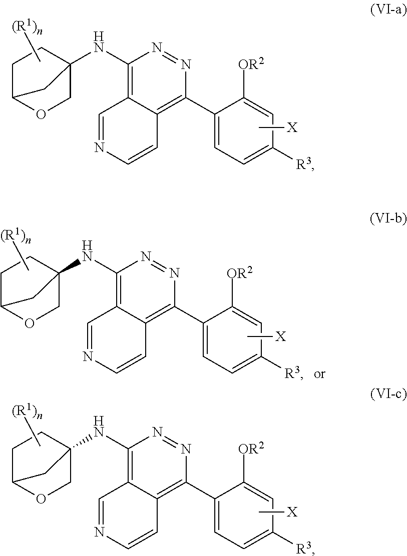
or a pharmaceutically acceptable salt or isotopically labeled derivative thereof.

23. The compound of any one of claims 1-15, wherein the compound is of Formula (VII-a), (VII-b), or (VII-c): 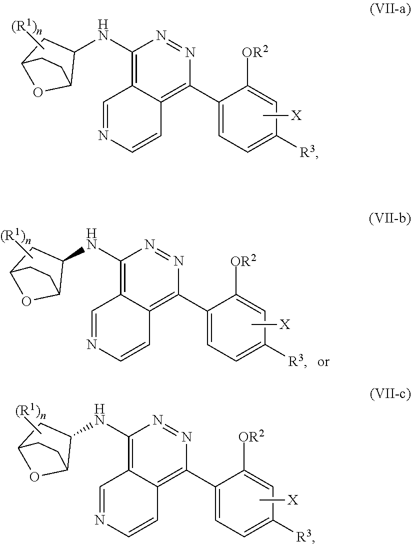
or a pharmaceutically acceptable salt or isotopically labeled derivative thereof.

24. The compound of any one of claims 1-15, wherein the compound is of Formula (VII-a1), (VII-b1), (VII-c1), (VII-a2), (VII-b2), or (VII-c2): 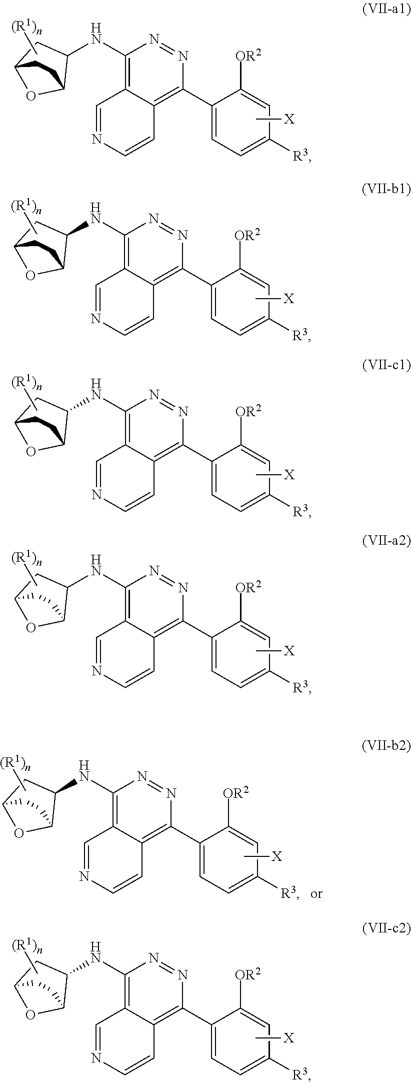
or a pharmaceutically acceptable salt or isotopically labeled derivative thereof.

25. The compound of any one of claim 1-14 or 16-24, or a pharmaceutically acceptable salt or isotopically labeled derivative thereof, wherein X is halogen or C₁₋₆ alkyl independently substituted with 0, 1, 2, or 3 halogen atoms, further wherein X is located at the ortho or meta position relative to the —OR² group.

26. The compound of claim 1 selected from the group consisting of a compound of Table 1, Table 2, or Table 3, or a pharmaceutically acceptable salt thereof or isotopically labeled derivative thereof.

27. The compound of any one of the preceding claims, or a pharmaceutically acceptable salt or isotopically labeled derivative thereof, wherein the compound has a Kpu,u>0.3.

28. The compound of any one of the preceding claims, or a pharmaceutically acceptable salt or isotopically labeled derivative thereof, wherein the compound has a Kpu,u>0.3 to about 10.

29. The compound of any one of claims 1-26, or a pharmaceutically acceptable salt or isotopically labeled derivative thereof, wherein the compound has a Kpu,u≤0.3.

30. A pharmaceutical composition comprising the compound of any one of the preceding claims, or a pharmaceutically acceptable salt or isotopically labeled derivative thereof, and one or more pharmaceutically acceptable excipients.

31. A method of modulating NLRP3, the method comprising administering to the subject a compound of any one of claims 1-29, or a pharmaceutically acceptable salt or isotopically labeled derivative thereof, or a pharmaceutical composition of claim 30.

32. A method of treating a disease or disorder, the method comprising administering to the subject a compound of any one of claims 1-29, or a pharmaceutically acceptable salt or isotopically labeled derivative thereof, or a pharmaceutical composition of claim 30.

33. The compound of any one of claims 1-29, or a pharmaceutically acceptable salt or isotopically labeled derivative thereof, or a pharmaceutical composition of claim 30, for use in treating a disease or disorder.

34. Use of the compound of any one of claims 1-29, or a pharmaceutically acceptable salt or isotopically labeled derivative thereof, or a pharmaceutical composition of claim 30, in the manufacture of a medicament, for the treatment of a disease or disorder.

35. Use of the compound of any one of claims 1-29, or a pharmaceutically acceptable salt or isotopically labeled derivative thereof, or a pharmaceutical composition of claim 30, for the treatment of a disease or disorder.

36. The method, compound, or use of any one of claims 32-35, wherein the disease or disorder is an NLRP3-related disease or disorder.

37. The method, compound, or use of any one of claims 31-36, wherein the subject is a human.

38. The method, compound, or use of any one of claims 32-37, wherein the disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an NLRP3-related disease.

39. The method, compound, or use of claim 38, wherein the disease or disorder of the central nervous system is Parkinson's disease, Alzheimer's disease, traumatic brain injury, spinal cord injury, amyotrophic lateral sclerosis, or multiple sclerosis.

40. The method, compound, or use of claim 38, wherein the kidney disease is an acute kidney disease, a chronic kidney disease, or a rare kidney disease.

41. The method, compound, or use of claim 38, wherein the skin disease is psoriasis, hidradenitis suppurativa (HS), or atopic dermatitis.

42. The method, compound, or use of claim 38, wherein the rheumatic disease is dermatomyositis, Still's disease, or juvenile idiopathic arthritis.

43. The method, compound, or use of claim 38, wherein the NIRP3-related disease is in a subject that has been determined to carry a germline or somatic non-silent mutation in NLRP3.

44. The method, compound, or use of claim 43, wherein the NLRP3-related disease is in a subject that has been determined to carry a germline or somatic non-silent mutation in NLRP3 is cryopyrin-associated autoinflammatory syndrome.

45. The method, compound, or use of claim 44, wherein the cryopyrin-associated autoinflammatory syndrome is familial cold autoinflammatory syndrome, Muckle-Wells syndrome, or neonatal onset multisystem inflammatory disease.

46. A method of preparing a compound of Formula (I): 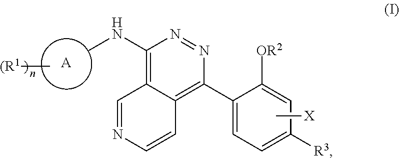
or a pharmaceutically acceptable salt or isotopically labeled derivative thereof; wherein Ring A, R¹, R², R³, X, and n are as defined in claim 1, the method comprising reacting an amine of formula (i), or salt or isotopically labeled derivative thereof, with a compound of formula (x), or salt or isotopically labeled derivative thereof:

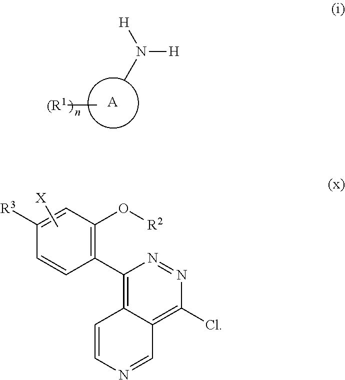

47. The method of claim 46, further comprising treating a compound of formula (ix), or salt or isotopically labeled derivative thereof, with a chlorinating agent to provide a compound of formula (x), salt or isotopically labeled derivative thereof:

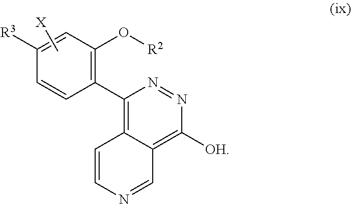

48. The method of claim 47, further comprising treating a compound of formula (viii), or salt or isotopically labeled derivative thereof, with a chlorinating agent, followed by condensation with hydrazine, to provide a compound of formula (ix), or salt or isotopically labeled derivative thereof:

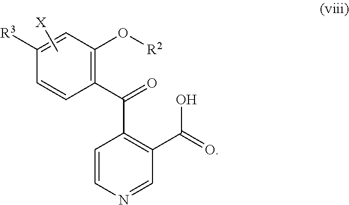

49. The method of claim 48, further comprising treating a 3,4-pyridinedicarboxylic acid anhydride of formula (vii), or salt or isotopically labeled derivative thereof, with a Grignard reagent of formula (xx), or salt or isotopically labeled derivative thereof, to provide a compound of formula (viii), or salt or isotopically labeled derivative thereof:

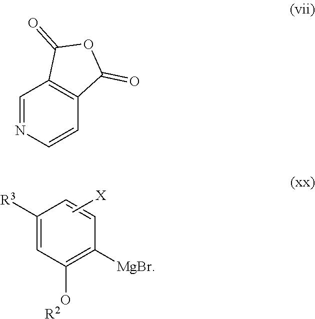

50. A method of preparing a compound of Formula (I): 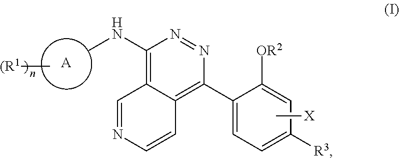
or a pharmaceutically acceptable salt or isotopically labeled derivative thereof; wherein Ring A, R¹, R², R³, X, and n are as defined in claim 1, the method comprising reacting a boronic acid or boronate of formula (iv), or salt or isotopically labeled derivative thereof, with a compound of formula (iii), or salt or isotopically labeled derivative thereof: 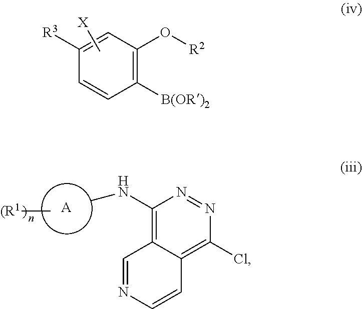
wherein R′ is H or C₁₋₆ alkyl, or two R′ groups are joined via a C₂-C₃ alkylene linker optionally substituted with one or more C₁₋₃ alkyl or C₁₋₃ haloalkyl.

51. The method of claim 50, further comprising treating a heteroaryl dichloride of formula (ii), or salt or isotopically labeled derivative thereof, with an amine of formula (i), or salt or isotopically labeled derivative thereof, to provide a compound of Formula (iii), or salt or isotopically labeled derivative

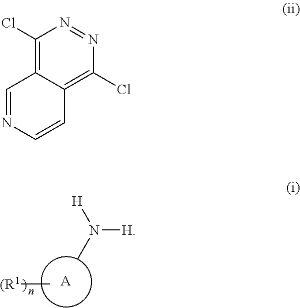

52. The method of claim 46 or 50, wherein R² is C₁-C₆ alkyl or —C(O)(C₁-C₆ alkyl), and wherein alkyl is optionally substituted with one or more halogen atoms, the method further comprising deprotecting the compound of Formula (I), or a pharmaceutically acceptable salt or isotopically labeled derivative thereof, to provide a compound of Formula (I), or a pharmaceutically acceptable salt or isotopically labeled derivative thereof, wherein R² is H.
